(19) United States
(12) Patent Application Publication
(10) Pub. No.: US 2014/0199682 A1
(43) Pub. Date: Jul. 17, 2014

(54) INFLUENZA NEUTRALIZING AGENTS

(71) Applicant: SEA LANE BIOTECHNOLOGIES, LLC, Altherton, CA (US)

(72) Inventors: Lawrence Horowitz, Atherton, CA (US); Ramesh R. Bhatt, Belmont, CA (US); Arun K. Kashyap, Newark, CA (US)

(73) Assignee: SEA LANE BIOTECHNOLOGIES, LLC, Atherton, CA (US)

(21) Appl. No.: 13/885,786

(22) PCT Filed: Nov. 17, 2011
(86) PCT No.: PCT/US11/01917

§ 371 Date: Mar. 18, 2014

Related U.S. Application Data
(60) Provisional application No. 61/414,794, filed on Nov. 17, 2010.

(51) Int. Cl. G01N 33/569; C07K 14/005; C07K 7/06

(52) U.S. Cl. CPC G01N 33/56983; C07K 7/06; C07K 14/005
USPC 435/5; 530/327; 530/324; 530/328

(57) ABSTRACT

The present invention concerns methods and means for identifying, producing, and engineering neutralizing agents against influenza A viruses, and to the neutralizing agents produced. In particular, the invention concerns neutralizing agents against various influenza A virus subtypes, and methods and means for making such agents.

### FIELD OF THE INVENTION

The present invention concerns methods for identifying, designing, producing, and engineering neutralizing agents against influenza A viruses, and to the neutralizing agents produced. The invention further concerns various uses of the agents produced, including the design and production of vaccines utilizing the binding sites of the neutralizing agents of the present invention on the target influenza A virus.

### BACKGROUND OF THE INVENTION

The flu is a contagious respiratory illness caused by influenza viruses. It causes mild to severe illness, and at times can lead to death. Annually, in the United States, influenza is contracted by 5-20% of the population, hospitalizing about 200,000, and causing the deaths of about 36,000.

Influenza viruses spread in respiratory droplets caused by coughing and sneezing, which are usually transmitted from person to person. Immunity to influenza surface antigens, particularly hemagglutinin, reduces the likelihood of infection and severity of disease if infection occurs. Although influenza vaccines are available, because a vaccine against one influenza virus type or subtype confers limited or no protection against another type or subtype of influenza, it is necessary to incorporate one or more new strains in each year's influenza vaccine.

Influenza viruses are segmented negative-strand RNA viruses and belong to the Orthomyxoviridae family. Influenza A virus consists of 9 structural proteins and codes additionally for one nonstructural NS1 protein with regulatory functions. The non-structural NS1 protein is synthesized in large quantities during the reproduction cycle and is localized in the cytosol and nucleus of the infected cells. The segmented nature of the viral genome allows the mechanism of genetic reassortment (exchange of genome segments) to take place during mixed infection of a cell with different viral strains. The influenza A virus may be further classified into various subtypes depending on the different hemagglutinin (HA) and neuraminidase (NA) viral proteins displayed on their surface. Influenza A virus subtypes are identified by two viral surface glycoproteins, hemagglutinin (HA or H) and neuraminidase (NA or N). Each influenza virus subtype is identified by its combination of H and N proteins. There are 16 known HA subtypes and 9 known NA subtypes. Influenza type A viruses can infect people, birds, pigs, horses, and other animals, but wild birds are the natural hosts for these viruses. Only some influenza A subtypes (i.e., H1N1 H1N2, and H3N2) are currently in circulation among people, but all combinations of the 16 H and 9 NA subtypes have been identified in avian species, especially in wild waterfowl and shorebirds. In addition, there is increasing evidence that H5 and H7 influenza viruses can also cause human illness.

The HA of influenza A virus comprises two structurally distinct regions, namely, a globular head region and a stem region. The globular head region contains a receptor binding site which is responsible for virus attachment to a target cell and participates in the hemagglutination activity of HA. The stem region contains a fusion peptide which is necessary for membrane fusion between the viral envelope and an endosomal membrane of the cell and thus relates to fusion activity (Wiley et al., Ann. Rev. Biochem., 56:365-394 (1987)).

A pandemic is a global disease outbreak. An influenza pandemic occurs when a new influenza A virus: (1) emerges for which there is little or no immunity in the human population, (2) begins to cause serious illness, and then (3) spreads easily person-to-person worldwide. During the 20^{th} century there have been three such influenza pandemics. First, in 1918, the “Spanish Flu” influenza pandemic caused at least 500,000 deaths in the United States and up to 40 million deaths worldwide. This pandemic was caused by influenza A H1N1 subtype. Second, in 1957, the “Asian Flu” influenza pandemic, caused by the influenza A H2N2 subtype, resulted in at least 70,000 deaths in the United States and 1-2 million deaths worldwide. Most recently in 1968 the “Hong Kong Flu” influenza pandemic, caused by the influenza A H3N2 subtype, resulted in about 34,000 U.S. deaths and 700,000 deaths worldwide.

In 1997, the first influenza A H5N1 cases were reported in Hong Kong. This was the first time that this type of avian virus directly infected humans, but a pandemic did not result because human to human transmission was not observed.

Lu et al., Resp. Res. 7:43 (2006) (doi: 10.1186/1465-992-7-43) report the preparation of anti-H5N1 IgGs from horses vaccinated with inactivated H5N1 virus, and of H5N1-specific F(ab′)₂ fragments, which were described to protect BALB/c mice infected with H5N1 virus.

Hanson et al., Resp. Res. 7:126 (doi: 10.1186/1465-9921-7-126) describe the use of a chimeric monoclonal antibody specific for influenza A H5 virus hemagglutinin for passive immunization of mice.

Neutralizing antibodies to influenza viruses are disclosed in U.S. Application Publication Nos. 20080014205 published on Jan. 17, 2008, and 20100040635 published on Feb. 18, 2010, as well as international PCT application no. PCT/US10/34604 filed on May 12, 2010.

In view of the severity of the respiratory illness caused by certain influenza A viruses, and the threat of a potential pandemic, there is a great need for effective preventative and treatment methods. The present invention addresses this need by providing influenza A neutralizing molecules against various H subtypes of the virus, including, without limitation, the H1, and H3 subtypes, and the H5 subtype of the influenza A virus. The invention further provides molecules capable of neutralizing more than one, and preferably all, isolates (strains) of a given subtype of the influenza A virus, including, without limitation, isolates obtained from various human and non-human species and isolates from victims and/or survivors of various influenza epidemics and/or pandemics.

Such crossreactive neutralizing molecules can be used for the prevention and/or treatment influenza virus infection, including passive immunization of infected or at risk populations in cases of epidemics or pandemics. Additionally, crossreactive antibodies can be used as a design guide for future vaccine discovery and an assessment tool for current vaccine clinical development.

Anti-influenza virus antibodies find utility in the prevention and treatment of diseases and disorders associated with influenza virus infection, and are useful for diagnostics, prophylaxis and treatment of disease.

### SUMMARY OF THE INVENTION

The present invention relates to methods of identifying a potential influenza virus neutralizing agent which mimics the binding site of an influenza neutralizing antibody to the influenza virus. For example, the methods rely upon amino acid modifications to the polypeptide components of an influenza neutralizing molecule. In one embodiment, the binding site is hemagglutinin (HA). In another embodiment, the neutralizing molecule is a C05 antibody. In one other embodiment, the agent mimics the binding site of an influenza virus A neutralizing molecule, wherein the molecule has one, two, or three hypervariable region sequences from a heavy chain selected from the group consisting of SEQ ID NO: 7, SEQ ID NO: 8, and SEQ ID NO: 9, or a functionally active fragment thereof. In one embodiment, the agent identified contains or does not contain the sequences shown in any one of SEQ ID NOS: 7-9, or variants thereof.

In another embodiment, the method includes the step of employing the variant amino acid sequences of at least one heavy chain hypervariable region sequence in rational drug design to design a potential influenza virus neutralizing agent which mimics the neutralizing molecule. In some embodiments, the hypervariable region sequence comprises a sequence selected from the group consisting of SEQ ID NO: 7; SEQ ID NO: 8, and SEQ ID NO: 9.

In another embodiment, the method includes the step of contacting the potential influenza virus neutralizing agent with an influenza virus to determine its capacity to act as a neutralizing agent. In one embodiment, the agent mimics a qualitative activity of the neutralizing antibody. In another embodiment, the agent has the ability to bind influenza virus.

In one other embodiment, the agent is a Surrobody, an antibody, or antibody fragment. In another embodiment, the agent comprises at least one heavy chain hypervariable region having a extended amino acid sequence as compared to its germline sequence. In one other embodiment, the at least one heavy chain hypervariable region is HVR-H1 and/or HVR-H3. In some embodiments, the HVR-H1 extended amino acid sequence comprises about 1 to about 5 amino acids. In other embodiments, the HVR-H3 extended amino acid sequence comprises about 1 to about 20 amino acids. In another embodiment, the HVR-H1 extended amino acid sequence is GESTL (SEQ ID NO: 30), or a variant thereof. In other embodiments, the HVR-H1 extended amino acid sequence is a variant of SEQ ID NO:7. In one other embodiment, the HVR-H3 extended amino acid sequence is HMSMQQVVSAGWERADLVGD (SEQ ID NO:31). In another embodiment, the HVR-H3 extended amino acid sequence is a variant of SEQ ID NO:9. In an embodiment, the agent comprises an amino acid modification adjacent to at least one heavy chain hypervariable region. In one other embodiment, the amino acid modification is N-terminal and/or C-terminal to the at least one heavy chain hypervariable region. In another embodiment, the heavy chain hypervariable region is HVR-H3.

In other embodiments, the agent is selected from a peptide mimetic, a fusion protein, an immunoadhesin, an antibody, a Surrobody, and a small molecule.

In another aspect, the present invention provides additional influenza virus neutralizing molecules. In one embodiment, the molecule comprises a) at least one HVR sequence selected from the group consisting of: (i) HVR-H1 comprising GESTLSYYAVS (SEQ ID NO:7); (ii) HVR-H2 comprising WLSIINAGGGDID (SEQ ID NO:8); (iii) HVR-H3 comprising AKHMSMQQVVSAGWERADLVGDAFD (SEQ ID NO:9), and b) at least one variant HVR, wherein the HVR comprises modification of at least one residue of the sequence depicted in SEQ ID NOS: 7, 8, or 9. In some embodiments, the G in a variant HVR-H1 is A. In other embodiments, the first Y residue in a variant HVR-H1 is F, (e.g., GESTLSFYAVS). In another embodiment, the molecule further includes at least one HVR sequence selected from the group consisting of: (i) IGAGYDVHWY (SEQ ID NO:13); (ii) LLIYDNNNRP (SEQ ID NO:14); (iii) QSYDNSLSGS (SEQ ID NO:15); (iv) IRKFLNWY (SEQ ID NO:16); (v) LLIYDASNLQ (SEQ ID NO:17); (vi) QQYDGLPF (SEQ ID NO:18); (vii) IRNSLNWY (SEQ ID NO:19); (viii) LLIHDASNLE (SEQ ID NO:20); and (ix) QQANSFPL (SEQ ID NO:21). In one other embodiment, the molecule further includes a surrogate light chain.

27.

### BRIEF DESCRIPTION OF THE DRAWINGS

FIG. 1 illustrates a strategy for increasing the reactive strengths towards two different targets (targets A and B), by recombining parallel discovery pools to generate/increase cross-reactivity. Each round of selection of the recombined antibody library increases the reactive strength of the resulting pool towards both targets.

FIG. 2 illustrates a strategy for increasing cross-reactivity to a target B while maintaining reactivity to a target A. First, a clone reactive with target A is selected, then a mutagenic library of the clones reactive with target A is prepared, and selection is performed as shown, yielding one or more antibody clones that show strong reactivity with both target A and target B.

FIG. 3 illustrates a representative mutagenesis method for generating a diverse multifunctional antibody collection by the “destinational mutagenesis” method.

FIG. 4 shows the binding ability of the 1286-C5 antibody to hemagglutinin antigens from H1, H3, and H9.

FIG. 5 shows the binding ability of the 1286-A11 antibody to hemagglutinin antigens from H1, H3, H5, and H9.

FIG. 6 illustrates a representative method for generating an amalgamated antibody library.

FIG. 7A-B illustrates the prophylactic effect of the C05 antibody against high titer lethal H3N2 viral challenge.

FIG. 7C-D show a therapeutic effect by the C05 antibody against lethal H3N2 viral challenge.

FIG. 7E shows the effect on survival of animals treated with the C05 antibody after a lethal H3N2 influenza infection (top panel), and a dose escalation study on day 3 post-infection (bottom panel).

FIG. 7F-G shows a prophylactic effect by the C05 antibody against lethal H1N1 viral challenge.

FIG. 7H shows a therapeutic effect by the C05 antibody against lethal H1N1 viral challenge.

FIG. 8 depicts the C05 antibody heavy chain sequence and shows the remarkably atypical length of heavy chain CDR 1.

FIG. 9 depicts the C05 antibody heavy chain sequence and shows the remarkably atypical length heavy chain CDR3.

FIG. 10 illustrates that C05 variants maintain recognition of H1 and H3 HA proteins.

FIG. 11 illustrates that C05 variants maintain recognition of H1 and H3 HA proteins.

FIG. 12 shows the binding of several different C05 Fab variants of CDR3.

FIG. 13 shows the binding of several different C05 Fab variants of CDR3.

### DETAILED DESCRIPTION

### A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), provides one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The terms “influenza A subtype” or “influenza A virus subtype” are used interchangeably, and refer to influenza A virus variants that are characterized by a hemagglutinin (H) viral surface protein, and thus are labeled by an H number, such as, for example, H1, H2, H3, H5, H9, and H12. In addition, the subtypes may be further characterized by a neuraminidase (N) viral surface protein, indicated by an N number, such as, for example, N1 and N2. As such, a subtype may be referred to by both H and N numbers, such as, for example, H1N1, H2N2, H3N2, H5N1, H5N2, and H9N2. The terms specifically include all strains (including extinct strains) within each subtype, which usually result from mutations and show different pathogenic profiles. Such strains will also be referred to as various “isolates” of a viral subtype, including all past, present and future isolates. Accordingly, in this context, the terms “strain” and “isolate” are used interchangeably. Subtypes contain antigens based upon an influenza A virus. The antigens may be based upon a hemagglutinin viral surface protein and can be designated as “HA antigen”. In some instances, such antigens are based on the protein of a particular subtype, such as, for example, an H1 subtype and an H3 subtype, which may be designated an H1 antigen and an H3 antigen, respectively.

The term “influenza” is used to refer to a contagious disease caused by an influenza virus.

In the context of the present invention, the term “binding molecule” is used in the broadest sense and includes any molecule comprising a polypeptide sequence that specifically binds to a target. The definition includes, without limitation, antibodies and antibody fragments, antibody-like molecules and fragments thereof, whether in monomeric or in a multimeric, such as homo- or heterodimeric, form. Multimeric binding molecules can retain their conformation through covalent and/or non-covalent interactions, and may be conjugated to each other and/or molecules or moieties, as long as they retain the requisite property of binding a target (e.g. an antigen in the case of antibodies).

A binding molecule that “specifically binds to” or is “specific for” a particular polypeptide or an epitope on a particular polypeptide is one that binds to that particular polypeptide or epitope on a particular polypeptide without substantially binding to any other polypeptide or polypeptide epitope.

The terms “conjugate,” “conjugated,” and “conjugation” refer to any and all forms of covalent or non-covalent linkage, and include, without limitation, direct genetic or chemical fusion, coupling through a linker or a cross-linking agent, and non-covalent association, for example through Van der Waals forces, or by using a leucine zipper.

The term “antibody” (Ab) is used in the broadest sense and includes polypeptides which exhibit binding specificity to a specific antigen as well as immunoglobulins and other antibody-like molecules which lack antigen specificity. Polypeptides of the latter kind are, for example, produced at low levels by the lymph system and, at increased levels, by myelomas. In the present application, the term “antibody” specifically covers, without limitation, monoclonal antibodies, polyclonal antibodies, and antibody fragments.

The term “C05”, “C5”, “C05 antibody” or “C05 antibody-like molecule” is used in the broadest sense and includes a binding molecule that specifically binds and neutralizes influenza virus. C05 may include one or more of the following heavy chain hypervariable region sequences SEQ ID NO:7, SEQ ID NO:8, and SEQ ID NO:9. C05 may also further include (i) one or more of the light chain hypervariable region sequences SEQ ID NO: 13, SEQ ID NO: 14, and SEQ ID NO: 15; (ii) one or more of the light chain hypervariable region sequences SEQ ID NO: 16, SEQ ID NO: 17, and SEQ ID NO: 18; or (iii) one or more of the light chain hypervariable region sequences SEQ ID NO: 19, SEQ ID NO: 20, and SEQ ID NO: 21. C05 may also include variants of the hypervariable region sequences described above. C05 molecules and other antibody and antibody-like molecules are further described in WO 2010/132604, incorporated herein by reference in its entirety.

The term “neutralizes”, “neutralizing”, “influenza neutralizing” or “influenza virus neutralizing” is used herein in the broadest sense and in reference to any molecule that inhibits a virus from replicatively infecting a target cell, irrespective of the mechanism by which neutralization is achieved. Neutralization can be achieved, for example, by inhibiting the attachment or adhesion of the virus to the cell surface, e.g., by engineering an molecule, such as an influenza neutralizing agent, that binds directly to, or close by, the site responsible for the attachment or adhesion of the virus. Neutralization can also be achieved by a molecule directed to the virion surface, which results in the aggregation of virions. Neutralization can further occur by inhibition of the fusion of viral and cellular membranes following attachment of the virus to the target cell, by inhibition of endocytosis, inhibition of progeny virus from the infected cell, and the like.

“Biological activity” is used herein in the context of a neutralizing agent that mimics the influenza virus neutralizing activity of an influenza neutralizing binding molecule, e.g., a C05 antibody, and can be identified by the screening assays disclosed herein refers, in part, to the ability of such agents to bind the influenza virus and/or to inhibit a virus from replicatively infecting a target cell, e.g., inhibiting hemagglutination.

In the context of the present invention, the term “influenza neutralizing agent” or “neutralizing agent” is used in the broadest sense and includes any molecule, including a binding molecule, comprising a polypeptide sequence that specifically binds to and neutralizes an influenza A virus. The definition includes, without limitation, neutralizing antibodies or antibody fragments; antibody-like molecules and fragments thereof, whether in monomeric or in a multimeric, such as homo- or heterodimeric, form; fragments, fusions or amino acid sequence variants of a neutralizing antibody, e.g., C05; polypeptides, peptides, peptide mimetics, antibody mimetics, and small molecules, including small organic molecules, etc. In a preferred embodiment, a neutralizing agent is any molecule that mimics a qualitative biological activity (as hereinabove defined) of a C05 antibody or C05 antibody-like molecule. The neutralizing agents of the present invention are not limited by the mechanism by which neutralization is achieved.

The terms “peptide mimetic” and “peptidomimetic” are used interchangeably, and refer to conformationally well defined peptide molecules, that mimic the structures and binding properties of an influenza virus recognition region (epitope) of an influenza virus neutralizing antibody described herein. Crystal structures of an antibody, e.g., C05, complexed with the virus allow for the identification and preparation of such peptide mimetics. The mimetics functionally mimic at least one variable region of an influenza neutralizing antibody or antibody-like molecule. The variable region is a region capable of neutralizing an influenza virus through interaction with the virus.

The terms “reduced oxidative potential” or “decreased oxidative heterogeneity potential” refer to an antibody containing a polypeptide with at least one amino acid substitution from an oxidizable amino acid to a non-oxidizable amino acid. The amino acid sequence may be a substitution for methionine. Antibody polypeptides may be selectively engineered to replace methionine amino acid residues with non-oxidizable amino acid residues thereby providing antibodies with reduced oxidative potential. For example, a methionine may be substituted with a leucine, a serine, or an alanine.

“Native antibodies” are usually heterotetrameric glycoproteins of about 150,000 daltons, composed of two identical light (L) chains and two identical heavy (H) chains. Each light chain is linked to a heavy chain by covalent disulfide bond(s), while the number of disulfide linkages varies between the heavy chains of different immunoglobulin isotypes. Each heavy and light chain also has regularly spaced intrachain disulfide bridges. Each heavy chain has, at one end, a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains, Chothia et al., J. Mol. Biol. 186:651 (1985); Novotny and Haber, Proc. Natl. Acad. Sci. U.S.A. 82:4592 (1985).

The term “variable” with reference to antibody chains is used to refer to portions of the antibody chains which differ extensively in sequence among antibodies and participate in the binding and specificity of each particular antibody for its particular antigen. Such variability is concentrated in three segments called hypervariable regions both in the light chain and the heavy chain variable domains. The more highly conserved portions of variable domains are called the framework region (FR). The variable domains of native heavy and light chains each comprise four FRs (FR1, FR2, FR3 and FR4, respectively), largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991), pages 647-669). The constant domains are not involved directly in binding an antibody to an antigen, but exhibit various effector functions, such as participation of the antibody in antibody-dependent cellular toxicity.

The term “hypervariable region” or “HVR” when used herein refers to the amino acid residues of an antibody which are responsible for antigen-binding. The hypervariable region comprises amino acid residues from a “complementarity determining region” or “CDR” (i.e., residues 30-36 (L1), 46-55 (L2) and 86-96 (L3) in the light chain variable domain and 30-35 (H1), 47-58 (H2) and 93-101 (H3) in the heavy chain variable domain; MacCallum et al., J Mol Biol. 1996. “Framework” or “FR” residues are those variable domain residues other than the hypervariable region residues as herein defined.

Depending on the amino acid sequence of the constant domain of their heavy chains, antibodies can be assigned to different classes. There are five major classes of antibodies IgA, IgD, IgE, IgG, and IgM, and several of these may be further divided into subclasses (isotypes), e.g., IgG1, IgG2, IgG3, IgG4, IgA, and IgA2.

The heavy-chain constant domains that correspond to the different classes of immunoglobulins are called α, δ, ε, γ, and μ, respectively.

The “light chains” of antibodies from any vertebrate species can be assigned to one of two clearly distinct types, called kappa (κ) and lambda (λ), based on the amino acid sequences of their constant domains.

The term “antibody fragment” is a portion of a full length antibody, generally the antigen binding or variable domain thereof. Examples of antibody fragments include, but are not limited to, Fab, Fab′, F(ab′)₂, and Fv fragments, linear antibodies, single-chain antibody molecules, diabodies, and multispecific antibodies formed from antibody fragments. Further examples of antibody fragments include, but are not limited to, scFv, (scFv)₂, dAbs (single-domain antibodies), and complementarity determining region (CDR) fragments, and minibodies, which are minimized variable domains whose two loops are amenable to combinatorial mutagenesis.

The term “monoclonal antibody” is used to refer to an antibody molecule synthesized by a single clone of B cells. The modifier “monoclonal” indicates the character of the antibody as being obtained from a substantially homogeneous population of antibodies, and is not to be construed as requiring production of the antibody by any particular method. Thus, monoclonal antibodies may be made by the hybridoma method first described by Kohler and Milstein, Nature 256:495 (1975); Eur. J. Immunol. 6:511 (1976), by recombinant DNA techniques, or may also be isolated from phage antibody libraries.

The term “polyclonal antibody” is used to refer to a population of antibody molecules synthesized by a population of B cells.

“Single-chain Fv” or “sFv” antibody fragments comprise the V_{H} and V_{L} domains of antibody, wherein these domains are present in a single polypeptide chain. Generally, the Fv polypeptide further comprises a polypeptide linker between the V_{H} and V_{L} domains which enables the sFv to form the desired structure for antigen binding. For a review of sFv see Plückthun in The Pharmacology of Monoclonal Antibodies, vol. 113, Rosenburg and Moore eds. Springer-Verlag, New York, pp. 269-315 (1994). Single-chain antibodies are disclosed, for example in WO 88/06630 and WO 92/01047.

The term “diabody” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444-6448 (1993).

The term “minibody” is used to refer to an scFv-CH3 fusion protein that self-assembles into a bivalent dimer of 80 kDa (scFv-CH3)₂.

The term “aptamer” is used herein to refer to synthetic nucleic acid ligands that bind to protein targets with high specificity and affinity. Aptamers are known as potent inhibitors of protein function.

A dAb fragment (Ward et al., Nature 341:544 546 (1989)) consists of a V_{H} domain or a VL domain.

As used herein the term “antibody binding regions” refers to one or more portions of an immunoglobulin or antibody variable region capable of binding an antigen(s). Typically, the antibody binding region is, for example, an antibody light chain (VL) (or variable region thereof), an antibody heavy chain (VH) (or variable region thereof), a heavy chain Fd region, a combined antibody light and heavy chain (or variable region thereof) such as a Fab, F(ab′)₂, single domain, or single chain antibody (scFv), or a full length antibody, for example, an IgG (e.g., an IgG1, IgG2, IgG3, or IgG4 subtype), IgA1, IgA2, IgD, IgE, or IgM antibody.

The term “bispecific antibody” refers to an antibody that shows specificities to two different types of antigens. The term as used herein specifically includes, without limitation, antibodies which show binding specificity for a target antigen and to another target that facilitates delivery to a particular tissue. Similarly, multi-specific antibodies have two or more binding specificities.

The expression “linear antibody” is used to refer to comprising a pair of tandem Fd segments (V_{H}-C_{H}1-V_{H}-C_{H}1) which form a pair of antigen binding regions. Linear antibodies can be bispecific or monospecific and are described, for example, by Zapata et al., Protein Eng. 8(10):1057-1062 (1995).

For the purposes of the present invention, the term “antibody-like molecule” includes any molecule, other than an antibody fragment as hereinabove defined, that is capable of binding to and neutralizing a viral antigen. The term specifically includes, without limitation, pre-B cell receptor (pre-BCR) like structures, referred to as “surrobodies,” including surrogate light chain (SLC) elements, as described, for example, in PCT Publication No. WO 2008/118970, published Oct. 2, 2008, and in Xu et al., Proc. Natl. Acad. Sci. USA, 105(31):10756-61 (2008). The SLC is a nondiversified heterodimer composed of the noncovalently associated Vpre-B and λ5 proteins. The VpreB chain is homologous to a Vλ Ig domain, and the λ5 chain is homologous to the Cλ domain of canonical antibodies, respectively. The heterodimeric SLC is covalently associated with the heavy chain in the pre-BCR complex by disulfide bonds between the Cλ domain and the first constant domain of the pre-BCR HC. A unique feature of the SLC is that the VpreB1 and the λ5 domains each have noncanonical peptide extensions. VpreB1 has an additional 21 residues on its C terminus, and λ5 has a 50-aa-long tail on its N terminus (see, e.g. Vettermann et al., Semin. Immunol. 18:44-55 (2006)). The surrobody structures specifically include, without limitation, the native trimeric pre-BCR-like functional unit of the pre-BCR, fusion of VpreB1 to λ5, and trimers that eliminated either the λ5 N-terminal 50 aa or the VpreB1 C-terminal 21 aa or both peptide extensions. In addition, chimeric constructs using the constant components of classical antibody light chains are specifically included within the definition of surrobodies.

Other representatives of “antibody-like molecules,” as defined herein, are similar structures comprising antibody surrogate κ light chain sequences, where κ light chain sequences are optionally partnered with another polypeptide, such as, for example, antibody heavy and/or light chain domain sequences. A κ-like B cell receptor (κ-like BCR) has been identified, utilizing a κ-like surrogate light chain (κ-like SLC) (Frances et al., EMBO J 13:5937-43 (1994); Thompson et al., Immunogenetics 48:305-11 (1998); Rangel et al., J Biol Chem 280:17807-14 (2005)). Rangel et al., J Biol Chem 280(18):17807-17814 (2005) report the identification and molecular characterization of a Vκ-like protein that is the product of an unrearranged Vκ gene, which turned out to the be identical to the cDNA sequence previously reported by Thompson et al., Immunogenetics 48:305-311 (1998). Whereas, Frances et al., EMBO J 13:5937-43 (1994) reported the identification and characterization of a rearranged germline JCk that has the capacity to associate with μ heavy chains at the surface of B cell precursors, thereby providing an alternative to the λ5 pathway for B cell development. It has been proposed that κ-like and λ-like pre-BCRs work in concert to promote light chain rearrangement and ensure the maturation of B cell progenitors. For a review, see McKeller and Martinez-Valdez Seminars in Immunology 18:4043 (2006).

The term “λ5” is used herein in the broadest sense and refers to any native sequence or variant λ5 polypeptide, specifically including, without limitation, native sequence human and other mammalian λ5 polypeptides, and variants formed by posttranslational modifications, as well a variants of such native sequence polypeptides.

The terms “‘variant VpreB polypeptide” and “a variant of a VpreB polypeptide” are used interchangeably, and are defined herein as a polypeptide differing from a native sequence VpreB polypeptide at one or more amino acid positions as a result of an amino acid modification. The “variant VpreB polypeptide,” as defined herein, will be different from a native antibody λ or K light chain sequence, or a fragment thereof. The “variant VpreB polypeptide” will preferably retain at least about 65%, or at least about 70%, or at least about 75%, or at least about 80%, or at least about 85%, or at least about 90%, or at least about 95%, or at least about 98% sequence identity with a native sequence VpreB polypeptide. In another preferred embodiment, the “variant VpreB polypeptide” will be less than 95%, or less than 90%, or less than 85%, or less than 80%, or less than 75%, or less than 70%, or less than 65%, or less than 60% identical in its amino acid sequence to a native antibody λ or K light chain sequence. Variant VpreB polypeptides specifically include, without limitation, VpreB polypeptides in which the non-Ig-like unique tail at the C-terminus of the VpreB sequence is partially or completely removed. The terms “variant λ5 polypeptide” and “a variant of a λ5 polypeptide” are used interchangeably, and are defined herein as a polypeptide differing from a native sequence λ5 polypeptide at one or more amino acid positions as a result of an amino acid modification. The “variant λ5 polypeptide,” as defined herein, will be different from a native antibody λ or K light chain sequence, or a fragment thereof. The “variant λ5 polypeptide” will preferably retain at least about 65%, or at least about 70%, or at least about 75%, or at least about 80%, or at least about 85%, or at least about 90%, or at least about 95%, or at least about 98% sequence identity with a native sequence λ5 polypeptide. In another preferred embodiment, the “‘variant λ5 polypeptide” will be less than 95%, or less than 90%, or less than 85%, or less than 80%, or less than 75%, or less than 70%, or less than 65%, or less than 60% identical in its amino acid sequence to a native antibody λ or κ light chain sequence. Variant λ5 polypeptides specifically include, without limitation. λ5 polypeptides in which the unique tail at the N-terminus of the λ5 sequence is partially or completely removed.

The term “VpreB sequence” is used herein to refer to the sequence of “VpreB,” as hereinabove defined, or a fragment thereof.

The term “λ5 sequence” is used herein to refers to the sequence of “λ5,” as hereinabove defined, or a fragment thereof.

The term “surrogate light chain sequence,” as defined herein, means any polypeptide sequence that comprises a “VpreB sequence” and/or a “λ5 sequence,” as hereinabove defined.

The terms “κ-like surrogate light chain variable domain,” “Vκ-like SLC,” and “Vκ-like” are used interchangeably, and refer to any native sequence polypeptide that is the product of an un-rearranged Vκ gene, and variants thereof. In one embodiment, variants of native sequence Vκ-like polypeptides comprise a C-terminal extension (tail) relative to antibody κ light chain sequences. In a particular embodiment, variants of native sequence Vκ-like polypeptides retain at least part, and preferably all, of the unique C-terminal extension (tail) that distinguishes the Vκ-like polypeptides from the corresponding antibody κ light chains. In another embodiment, the C-terminal tail of the variant Vκ-like polypeptide is a sequence not naturally associated with the rest of the sequence. In the latter embodiment, the difference between the C-terminal tail naturally present in the native Vκ-like sequence and the variant sequence may result from one or more amino acid alterations (substitutions, insertions, deletions, and/or additions), or the C-terminal tail may be identical with a tail present in nature in a different Vκ-like protein. The Vκ-like polypeptides may contain amino acid alterations in regions corresponding to one or more of antibody κ light chain CDR1, CDR2 and CDR3 sequences. In all instances, the variants can, and preferably do, include a C-terminal extension of at least four, or at least five, or at least six, or at least seven, or at least eight, or at least nine, or at least ten amino acids, preferably 4-100, or 4-90, or 4-80, or 4-70, or 4-60, or 4-50, or 4-45, or 4-40, or 4-35, or 4-30, or 4-25, or 4-20, or 4-15, or 4-10 amino acid residues relative to a native antibody κ light chain variable region sequence. As defined herein, Vκ-like polypeptide variant will be different from a native antibody κ or light chain sequence or a fragment thereof, and will preferably retain at least about 65%, or at least about 70%, or at least about 75%, or at least about 80%, or at least about 85%, or at least about 90%, or at least about 95%, or at least about 98% sequence identity with a native sequence Vκ polypeptide. In another preferred embodiment, the Vκ-like polypeptide variant will be less than 95%, or less than 90%, or less than 85%, or less than 80%, or less than 75%, or less than 70%, or less than 65%, or less than 60%, or less than 55%, or less than 50%, or less than 45%, or less than 40% identical in its amino acid sequence to a native antibody λ or K light chain sequence. In other embodiments, the sequence identity is between about 40% and about 95%, or between about 45% and about 90%, or between about 50% and about 85%, or between about 55% and about 80%, or between about 60% and about 75%, or between about 60% and about 80%, or between about 65% and about 85%, or between about 65% and about 90%, or between about 65% and about 95%. In all embodiments, preferably the Vκ-like polypeptides are capable of binding to a target.

The terms “JCκ” and “JCκ-like” are used interchangeably, and refer to native sequence polypeptides that include a portion identical to a native sequence κJ-constant (C) region segment and a unique N-terminal extension (tail), and variants thereof. In one embodiment, variants of native sequence JCκ-like polypeptides comprise an N-terminal extension (tail) that distinguishes them from an antibody JC segment. In a particular embodiment, variants of native sequence JCκ-like polypeptides retain at least part, and preferably all, of the unique N-terminal extension (tail) that distinguishes the JCκ-like polypeptides from the corresponding antibody κ light chain JC segments. In another embodiment, the N-terminal tail of the variant JCκ-like polypeptide is a sequence not naturally associated with the rest of the sequence. In the latter embodiment, the difference between the N-terminal tail naturally present in the native JCκ-like sequence and the variant sequence may result from one or more amino acid alterations (substitutions, insertions, deletions, and/or additions), or the N-terminal tail may be identical with a tail present in nature in a different JCκ-like protein. Variants of native sequence JCκ-like polypeptides may contain one or more amino acid alterations in the part of the sequence that is identical to a native antibody κ variable domain JC sequence. In all instances, the variants can, and preferably do, include an N-terminal extension (unique N-terminus) of at least four, or at least five, or at least six, or at least seven, or at least eight, or at least nine, or at least ten amino acids, preferably 4-100, or 4-90, or 4-80, or 4-70, or 4-60, 4-50, or 4-45, or 4-40, or 4-35, or 4-30, or 4-25, or 4-20, or 4-15, or 4-10 amino acid residues relative to a native antibody κ light chain JC sequence. The JCκ-like polypeptide variant, as defined herein, will be different from a native antibody λ or κ light chain JC sequence, or a fragment thereof, and will preferably retain at least about 65%, or at least about 70%, or at least about 75%, or at least about 80%, or at least about 85%, or at least about 90%, or at least about 95%, or at least about 98% sequence identity with a native sequence JC polypeptide. In another preferred embodiment, the JCκ-like polypeptide variant will be less than 95%, or less than 90%, or less than 85%, or less than 80%, or less than 75%, or less than 70%, or less than 65%, or less than 60% identical in its amino acid sequence to a native antibody λ or κ light chain JC sequence. In other embodiments, the sequence identity is between about 40% and about 95%, or between about 45% and about 90%, or between about 50% and about 85%, or between about 55% and about 80%, or between about 60% and about 75%, or between about 60% and about 80%, or between about 65% and about 85%, or between about 65% and about 90%, or between about 65% and about 95%.

Percent amino acid sequence identity may be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

The term “fusion” is used herein to refer to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini.

As used herein, the terms “peptide,” “polypeptide” and “protein” all refer to a primary sequence of amino acids that are joined by covalent “peptide linkages.” In general, a peptide consists of a few amino acids, typically from about 2 to about 50 amino acids, and is shorter than a protein. The term “polypeptide,” as defined herein, encompasses peptides and proteins.

The term “amino acid” or “amino acid residue” typically refers to an amino acid having its art recognized definition such as an amino acid selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val) although modified, synthetic, or rare amino acids may be used as desired. Thus, modified and unusual amino acids listed in 37 CFR 1.822(b)(4) are specifically included within this definition and expressly incorporated herein by reference. Amino acids can be subdivided into various sub-groups. Thus, amino acids can be grouped as having a nonpolar side chain (e.g., Ala, Cys, Ile, Leu, Met, Phe, Pro, Val); a negatively charged side chain (e.g., Asp, Glu); a positively charged side chain (e.g., Arg, His, Lys); or an uncharged polar side chain (e.g., Asn, Cys, Gln, Gly, His, Met, Phe, Ser, Thr, Trp, and Tyr). Amino acids can also be grouped as small amino acids (Gly, Ala), nucleophilic amino acids (Ser, His, Thr, Cys), hydrophobic amino acids (Val, Leu, Ile, Met, Pro), aromatic amino acids (Phe, Tyr, Trp, Asp, Glu), amides (Asp, Glu), and basic amino acids (Lys, Arg).

The term “polynucleotide(s)” refers to nucleic acids such as DNA molecules and RNA molecules and analogues thereof (e.g., DNA or RNA generated using nucleotide analogues or using nucleic acid chemistry). As desired, the polynucleotides may be made synthetically, e.g., using art-recognized nucleic acid chemistry or enzymatically using, e.g., a polymerase, and, if desired, be modified. Typical modifications include methylation, biotinylation, and other art-known modifications. In addition, the nucleic acid molecule can be single-stranded or double-stranded and, where desired, linked to a detectable moiety.

The term “variant” with respect to a reference polypeptide refers to a polypeptide that possesses at least one amino acid mutation or modification (i.e., alteration) as compared to a native polypeptide. Variants generated by “amino acid modifications” can be produced, for example, by substituting, deleting, truncating, inserting and/or chemically modifying at least one amino acid in the native amino acid sequence.

An “amino acid modification” refers to a change in the amino acid sequence of a predetermined amino acid sequence. Exemplary modifications include an amino acid substitution, insertion and/or deletion.

An “amino acid modification at a specified position,” refers to the substitution or deletion of the specified residue, or the insertion of at least one amino acid residue adjacent the specified residue. By insertion “adjacent” a specified residue is meant insertion within one to two residues thereof. The insertion may be N-terminal or C-terminal to the specified residue.

An “amino acid substitution” refers to the replacement of at least one existing amino acid residue in a predetermined amino acid sequence with another different “replacement” amino acid residue. The replacement residue or residues may be “naturally occurring amino acid residues” (i.e. encoded by the genetic code) and selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val). Substitution with one or more non-naturally occurring amino acid residues is also encompassed by the definition of an amino acid substitution herein.

A “non-naturally occurring amino acid residue” refers to a residue, other than those naturally occurring amino acid residues listed above, which is able to covalently bind adjacent amino acid residues(s) in a polypeptide chain. Examples of non-naturally occurring amino acid residues include norleucine, ornithine, norvaline, homoserine and other amino acid residue analogues such as those described in Ellman et al. Meth. Enzym. 202:301 336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA.

An “amino acid insertion” refers to the incorporation of at least one amino acid into a predetermined amino acid sequence. While the insertion will usually consist of the insertion of one or two amino acid residues, the present application contemplates larger “peptide insertions”, e.g. insertion of about three to about five or even up to about ten amino acid residues. The inserted residue(s) may be naturally occurring or non-naturally occurring as disclosed above.

An “amino acid deletion” refers to the removal of at least one amino acid residue from a predetermined amino acid sequence.

The term “mutagenesis” refers to, unless otherwise specified, any art recognized technique for altering a polynucleotide or polypeptide sequence. Preferred types of mutagenesis include error prone PCR mutagenesis, saturation mutagenesis, or other site directed mutagenesis.

“Site-directed mutagenesis” is a technique standard in the art, and is conducted using a synthetic oligonucleotide primer complementary to a single-stranded phage DNA to be mutagenized except for limited mismatching, representing the desired mutation. Briefly, the synthetic oligonucleotide is used as a primer to direct synthesis of a strand complementary to the single-stranded phage DNA, and the resulting double-stranded DNA is transformed into a phage-supporting host bacterium. Cultures of the transformed bacteria are plated in top agar, permitting plaque formation from single cells that harbor the phage. Theoretically, 50% of the new plaques will contain the phage having, as a single strand, the mutated form; 50% will have the original sequence. Plaques of interest are selected by hybridizing with kinased synthetic primer at a temperature that permits hybridization of an exact match, but at which the mismatches with the original strand are sufficient to prevent hybridization. Plaques that hybridize with the probe are then selected, sequenced and cultured, and the DNA is recovered.

The term “antibody repertoire” is used herein in the broadest sense and refers to a collection of antibodies or antibody fragments which can be used to screen for a particular property, such as binding ability, binding specificity, ability of gastrointestinal transport, stability, affinity, and the like. The term specifically includes antibody libraries, including all forms of combinatorial libraries, such as, for example, antibody phage display libraries, including, without limitation, single-chain Fv (scFv) and Fab antibody phage display libraries from any source, including naïve, synthetic and semi-synthetic libraries.

Similarly, a “repertoire of antibody-like molecules” (as hereinabove defined) refers to a collection of such molecules which can be used to screen for a particular property, such as binding ability, binding specificity, ability of gastrointestinal transport, stability, affinity, and the like. The term specifically includes surrobody libraries and libraries of κ-like light chain constructs (as hereinabove defined), including all forms of combinatorial libraries, such as, for example, phage display libraries. Combinatorial surrobody libraries are disclosed, for example, in Xu et al., (2008), supra.

A “phage display library” is a protein expression library that expresses a collection of cloned protein sequences as fusions with a phage coat protein. Thus, the phrase “phage display library” refers herein to a collection of phage (e.g., filamentous phage) wherein the phage express an external (typically heterologous) protein. The external protein is free to interact with (bind to) other moieties with which the phage are contacted. Each phage displaying an external protein is a “member” of the phage display library.

An “antibody phage display library” refers to a phage display library that displays antibodies or antibody fragments. The antibody library includes the population of phage or a collection of vectors encoding such a population of phage, or cell(s) harboring such a collection of phage or vectors. The library can be monovalent, displaying on average one single-chain antibody or antibody fragment per phage particle, or multi-valent, displaying, on average, two or more antibodies or antibody fragments per viral particle. The term “antibody fragment” includes, without limitation, single-chain Fv (scFv) fragments and Fab fragments. Preferred antibody libraries comprise on average more than 10⁶, or more than 10⁷, or more than 10⁸, or more than 10⁹ different members.

The term “filamentous phage” refers to a viral particle capable of displaying a heterogenous polypeptide on its surface, and includes, without limitation, f1, fd, Pf1, and M13. The filamentous phage may contain a selectable marker such as tetracycline (e.g., “fd-tet”). Various filamentous phage display systems are well known to those of skill in the art (see, e.g., Zacher et al., Gene 9:127-140 (1980), Smith et al., Science 228:1315-1317 (1985); and Parmley and Smith, Gene 73:305-318 (1988)).

The term “panning” is used to refer to the multiple rounds of screening process in identification and isolation of phages carrying compounds, such as antibodies, with high affinity and specificity to a target.

The term “non-human animal” as used herein includes, but is not limited to, mammals such as, for example, non-human primates, rodents (e.g., mice and rats), and non-rodent animals, such as, for example, rabbits, pigs, sheep, goats, cows, pigs, horses and donkeys. It also includes birds (e.g., chickens, turkeys, ducks, geese and the like). The term “non-primate animal” as used herein refers to mammals other than primates, including but not limited to the mammals specifically listed above.

The phrase “functionally different antibodies,” and grammatical variants thereof, are used to refer to antibodies that differ from each other in at least one property, including, without limitation, binding specificity, binding affinity, and any immunological or biological function, such as, for example, ability to neutralize a target, extent or quality of biological activity, etc.

The phrase “conserved amino acid residues” is used to refer to amino acid residues that are identical between two or more amino acid sequences aligned with each other.

The term “epitope” as used herein, refers to a sequence of at least about 3 to 5, preferably at least about 5 to 10, or at least about 5 to 15 amino acids, and typically not more than about 500, or about 1,000 amino acids, which define a sequence that by itself, or as part of a larger sequence, binds to an antibody generated in response to such sequence. An epitope is not limited to a polypeptide having a sequence identical to the portion of the parent protein from which it is derived. Indeed, viral genomes are in a state of constant change and exhibit relatively high degrees of variability between isolates. Thus the term “epitope” encompasses sequences identical to the native sequence, as well as modifications, such as deletions, substitutions and/or insertions to the native sequence. Generally, such modifications are conservative in nature but non-conservative modifications are also contemplated. The term specifically includes “mimotopes,” i.e. sequences that do not identify a continuous linear native sequence or do not necessarily occur in a native protein, but functionally mimic an epitope on a native protein. The term “epitope” specifically includes linear and conformational epitopes.

The “active site” of an influenza virus neutralizing antibody comprises a heavy chain variable domain loop. The active site may comprise the CDR3 loop or a combination of the CDR3 and CDR1 loops. These loops and several other residues around the active site may be shown by mutational analysis to be the key structural determinants of the influenza virus neutralizing activity, e.g., mutations in C05.

As used herein, “solvent accessible position” refers to a position of an amino acid residue in the variable regions of the heavy and light chains of the antibody or antigen binding fragment that is determined, based on structure, ensemble of structures and/or modeled structure of the antibody or antigen binding fragment, as potentially available for solvent access and/or contact with a molecule, such as an antibody-specific antigen, e.g., C05 binding site on an influenza virus. These positions are typically found in the CDRs and on the exterior of the influenza virus. The solvent accessible positions of an antibody or antigen binding fragment, as defined herein, can be determined using any of a number of algorithms known in the art. Preferably, solvent accessible positions are determined using coordinates from a 3-dimensional model of an antibody or antibody fragment bound to its antigen, preferably using a computer program such as the InsightII program (Accelrys, San Diego, Calif.). Solvent accessible positions can also be determined using algorithms known in the art (e.g., Lee and Richards (1971) J. Mol. Biol. 55, 379 and Connolly (1983) J. Appl. Cryst. 16, 548). Determination of solvent accessible positions can be performed using software suitable for protein modeling and 3-dimensional structural information obtained from an antibody. Software that can be utilized for these purposes includes SYBYL Biopolymer Module software (Tripos Associates). Generally and preferably, where an algorithm (program) requires a user input size parameter, the “size” of a probe which is used in the calculation is set at about 1.4 Angstrom or smaller in radius. In addition, determination of solvent accessible regions and area methods using software for personal computers has been described by Pacios (1994) Comput. Chem. 18(4): 377-386.

The term “binding pocket” or “binding domain” refers to a region of a molecule or molecular complex, which, as a result of its shape, favorably associates with another chemical entity. The term “pocket” includes, but is not limited to, a cleft, channel or site. The shape of a binding pocket may be largely pre-formed before binding of a chemical entity, may be formed simultaneously with binding of a chemical entity thereto, or may be formed by the binding of another chemical entity thereto to a different binding pocket of the molecule, which in turn induces a change in shape of the binding pocket.

The term “generating a three-dimensional structure” or “generating a three-dimensional representation” refers to converting the lists of structure coordinates into structural models or graphical representation in three-dimensional space. This can be achieved through commercially or publicly available software. A model of a three-dimensional structure of a molecule or molecular complex can thus be constructed on a computer screen by a computer that is given the structure coordinates and that comprises the correct software. The three-dimensional structure may be displayed or used to perform computer modeling or fitting operations. In addition, the structure coordinates themselves, without the displayed model, may be used to perform computer-based modeling and fitting operations.

The term “crystallization solution” refers to a solution that promotes crystallization comprising at least one agent, including a buffer, one or more salts, a precipitating agent, one or more detergents, sugars or organic compounds, lanthanide ions, a poly-ionic compound and/or a stabilizer.

“Therapeutically effective amount” is the amount of an “influenza neutralizing agent” which is required to achieve a measurable improvement in the state, e.g. pathology, of the target disease or condition, such as, for example, an influenza virus infection.

### B. General Techniques

Techniques for performing the methods of the present invention are well known in the art and described in standard laboratory textbooks, including, for example, Ausubel et al., Current Protocols of Molecular Biology, John Wiley and Sons (1997); Molecular Cloning: A Laboratory Manual, Third Edition, J. Sambrook and D. W. Russell, eds., Cold Spring Harbor, N.Y., USA, Cold Spring Harbor Laboratory Press, 2001; Antibody Phage Display: Methods and Protocols, P. M. O'Brian and R. Aitken, eds., Humana Press, In: Methods in Molecular Biology, Vol. 178; Phage Display: A Laboratory Manual, C. F. Barbas III et al. eds., Cold Spring Harbor, N.Y., USA, Cold Spring Harbor Laboratory Press, 2001; and Antibodies, G. Subramanian, ed., Kluwer Academic, 2004. Mutagenesis can, for example, be performed using site-directed mutagenesis (Kunkel et al., Proc. Natl. Acad. Sci USA 82:488-492 (1985)).

The present invention concerns methods for identifying, designing, producing, and engineering neutralizing agents against influenza A viruses, and to the neutralizing agents produced. In general, the agents identified will be viral antigen neutralizing molecules. In one aspect, the viral antigen neutralizing molecules of the present invention are antibodies, which are typically selected using antibody or diversified polypeptide libraries. In the following description, the invention is illustrated with reference to certain types of antibody libraries, but the invention is not limited to the use of any particular type of antibody or diversified polypeptide library. Recombinant monoclonal antibody libraries can be based on immune fragments or naïve fragments. Antibodies from immune antibody libraries are typically constructed with V_{H} and V_{L} gene pools that are cloned from source B cells into an appropriate vector for expression to produce a random combinatorial library, which can subsequently be selected for and/or screened. Other types of libraries may be comprised of antibody fragments from a source of genes that is not explicitly biased for clones that bind to an antigen. Thus, naïve antibody libraries derive from natural, unimmunized, rearranged V genes. Synthetic antibody libraries are constructed entirely by in vitro methods, introducing areas of complete or tailored degeneracy into the CDRs of one or more V genes. Semi-synthetic libraries combine natural and synthetic diversity, and are often created to increase natural diversity while maintaining a desired level of functional diversity. Thus, such libraries can, for example, be created by shuffling natural CDR regions (Soderlind et al., Nat. Biotechnol. 18:852-856 (2000)), or by combining naturally rearranged CDR sequences from human B cells with synthetic CDR1 and CDR2 diversity (Hoet et al., Nat. Biotechnol. 23:455-38 (2005)). The methods of the present invention for identifying potential influenza virus neutralizing agent which mimic the binding site of an influenza virus A neutralizing molecule, e.g., C05, encompass the use of naïve, synthetic and semi-synthetic antibody libraries, or any combination thereof, as well as any method described herein.

Similarly, the methods of the present invention are not limited by any particular technology used for the display of antibodies. Although the invention is illustrated with reference to phage display, antibodies of the present invention can also be identified by other display and enrichment technologies. Antibody fragments have been displayed on the surface of filamentous phage that encode the antibody genes (Hoogenboom and Winter J. Mol. Biol., 222:381 388 (1992); McCafferty et al., Nature 348(6301):552 554 (1990); Griffiths et al. EMBO J., 13(14):3245-3260 (1994)). For a review of techniques for selecting and screening antibody libraries see, e.g., Hoogenboom, Nature Biotechnol. 23(9):1105-1116 (2005). In addition, there are systems known in the art for display of heterologous proteins and fragments thereof on the surface of Escherichia coli (Agterberg et al., Gene 88:37-45 (1990); Charbit et al., Gene 70:181-189 (1988); Francisco et al., Proc. Natl. Acad. Sci. USA 89:2713-2717 (1992)), and yeast, such as Saccharomyces cerevisiae (Boder and Wittrup, Nat. Biotechnol. 15:553-557 (1997); Kieke et al., Protein Eng. 10:1303-1310 (1997)). Other known display techniques include ribosome or mRNA display (Mattheakis et al., Proc. Natl. Acad. Sci. USA 91:9022-9026 (1994); Hanes and Pluckthun, Proc. Natl. Acad. Sci. USA 94:4937-4942 (1997)), DNA display (Yonezawa et al., Nucl. Acid Res. 31(19):e118 (2003)); microbial cell display, such as bacterial display (Georgiou et al., Nature Biotech. 15:29-34 (1997)), display on mammalian cells, spore display (Isticato et al., J. Bacteriol. 183:6294-6301 (2001); Cheng et al., Appl. Environ. Microbiol. 71:3337-3341 (2005) and co-pending provisional application Ser. No. 60/865,574, filed Nov. 13, 2006), viral display, such as retroviral display (Urban et al., Nucleic Acids Res. 33:e35 (2005), display based on protein-DNA linkage (Odegrip et al., Proc. Acad. Natl. Sci. USA 101:2806-2810 (2004); Reiersen et al., Nucleic Acids Res. 33:e10 (2005)), and microbead display (Sepp et al., FEBS Lett. 532:455-458 (2002)).

### C. Detailed Description of Preferred Embodiments

In one aspect, the present invention concerns the design, selection, production and use of influenza neutralizing agents that can neutralize more than one subtype and/or more than one isolate of an influenza A virus, binding to a hemagglutinin (HA) antigen of the virus.

The virions of influenza A virus contain 8 segments of linear negative-sense single stranded RNA. The total genome length is 13600 nucleotides, and the eight segments are 2350 nucleotides; 2350 nucleotides; of 2250 nucleotides; 1780 nucleotides; 1575 nucleotides; 1420 nucleotides; 1050 nucleotides; and 900 nucleotides, respectively, in length. Host specificity and attenuation of influenza A virus have been attributed to viral hemagglutinin (H, HA), nucleoprotein (NP), matrix (M), and non-structural (NS) genes individually or in combinations of viral genes (see, e.g., Rogers et al., Virology. 127:361-373 (1983); Scholtissek et al., Virology 147:287-294 (1985); Snyder et al., J. Clin. Microbiol. 24:467-469 (1986); Tian et al., J. Virol. 53:771-775 (1985); Treanor et al., Virology 171:1-9 (1989).

Nucleotide and amino acid sequences of influenza A viruses and their surface proteins, including hemagglutinins and neuraminidase proteins, are available from GenBank and other sequence databases, such as, for example, the Influenza Sequence Database maintained by the Theoretical Biology and Biophysics Group of Los Alamos National Laboratory. The amino acid sequences of 15 known H subtypes of the influenza A virus hemagglutinin (H1-H15) are shown in U.S. Application Publication No. 20080014205, published on Jan. 17, 2008, incorporated herein by reference in its entirety. An additional influenza A virus hemagglutinin subtype (H16) was isolated recently from black-headed gulls in Sweden, and reported by Fouchier et al., J. Virol. 79(5):2814-22 (2005). A large variety of strains of each H subtype are also known. For example, the sequence of the HA protein designated H5 A/Hong Kong/156/97 was determined from an influenza A H5N1 virus isolated from a human in Hong Kong in May 1997, and is shown in comparison with sequences of several additional strains obtained from other related H5N1 isolates in Suarez et al., J. Virol. 72:6678-6688 (1998).

The structure of the catalytic and antigenic sites of influenza virus neuraminidase have been published by Colman et al., Nature 303:41-4 (1983), and neuraminidase sequences are available from GenBank and other sequence databases.

It has been known that virus-specific antibodies resulting from the immune response of infected individuals typically neutralize the virus via interaction with the viral hemagglutinin (Ada et al., Curr. Top. Microbiol. Immunol. 128:1-54 (1986); Couch et al., Annu. Rev. Micobiol. 37:529-549 (1983)). The three-dimensional structures of influenza virus hemagglutinins and crystal structures of complexes between influenza virus hemagglutinins and neutralizing antibodies have also been determined and published, see, e.g., Wilson et al., Nature 289:366-73 (1981); Ruigrok et al., J. Gen. Virol. 69 (Pt 11):2785-95 (1988); Wrigley et al., Virology 131(2):308-14 (1983); Daniels et al., EMBO J. 6:1459-1465 (1987); and Bizebard et al., Nature 376:92-94 (2002).

According to the present invention, influenza virus neutralizing agents with the desired properties are identified based upon the characterization of an antibody or antibody-like molecule known to bind to and neutralize the virus, e.g., C05 antibody.

Human Influenza Neutralizing Binding Molecules

In one aspect, the agents identified by the methods provided herein are binding molecules comprising hypervariable regions from heavy chain and light chain polypeptides. In one embodiment, the binding molecule comprises one, two, or three hypervariable region sequences from a heavy chain selected from the group consisting of SEQ ID NO:7, SEQ ID NO:8, and SEQ ID NO:9, or a functionally active fragment thereof. In another embodiment, the binding molecule comprises all hypervariable region sequences SEQ ID NO:7, SEQ ID NO:8, and SEQ ID NO:9. In one other embodiment, the binding molecule is a binding molecule which is capable of binding a target when associated with a light chain. In one embodiment, the light chain or binding molecule comprises one, two or three hypervariable sequences of the polypeptide sequence of SEQ ID NO:13, SEQ ID NO:14, and SEQ ID NO:15. In another embodiment, the light chain or binding molecule comprises one, two or three hypervariable sequences of the polypeptide sequence of SEQ ID NO:16, SEQ ID NO:17, and SEQ ID NO:18. In one other embodiment, the light chain or binding molecule comprises one, two or three hypervariable sequences of the polypeptide sequence of SEQ ID NO:19, SEQ ID NO:20, and SEQ ID NO:21. In another embodiment, the light chain or binding molecule comprises all hypervariable region sequences SEQ ID NO:13, SEQ ID NO:14, and SEQ ID NO:15. In another embodiment, the light chain or binding molecule comprises all hypervariable region sequences SEQ ID NO:16, SEQ ID NO:17, and SEQ ID NO:18. In one other embodiment, the light chain or binding molecule comprises all hypervariable region sequences SEQ ID NO:19, SEQ ID NO:20, and SEQ ID NO:21. In a preferred embodiment, the binding molecule is the C05 antibody.

In one embodiment, the binding molecule is an antibody. In another embodiment, the binding molecule is a Surrobody.

In another aspect, the present invention provides binding molecules comprising a VpreB sequence and/or a λ5 sequence. In one embodiment, the binding molecule comprises a polypeptide comprising a VpreB sequence and/or a λ5 sequence. In another embodiment, the binding molecule further comprises a polypeptide comprising a VpreB sequence fused to a λ5 sequence. In one other embodiment, the binding molecule further comprises a κ-like surrogate light chain (SLC) construct comprising a Vκ-like and/or a JCκ sequence.

In some embodiments, the binding molecules (i) neutralize more than one subtype and/or more than one isolate of an influenza A virus, (ii) bind to a hemagglutinin (HA) antigen of the virus, and (iii) inhibit hemagglutination. In another embodiment, the binding molecule which neutralizes at least one of the H1 and H3 influenza A virus subtypes. In one embodiment, the binding molecule neutralizes the H1 and H3 influenza A virus subtypes. In another embodiment, the binding molecule prevents the globular head region of the influenza A virus from binding the surface of a cell. In one other embodiment, the binding molecule prevents the influenza A virus from attaching to a cell to be infected. In another embodiment, the binding molecule binds to an H1 HA antigen. In one embodiment, the binding molecule binds to at least one additional HA antigen. In another embodiment, the additional HA antigen is H3. In another embodiment, the binding molecule binds to an H2 HA antigen. In some other embodiment, the binding molecule binds to H9 HA antigen. In another embodiment, the binding molecule binds to H12 HA antigen.

In one embodiment, the present invention provides an antibody comprising a heavy chain, the heavy chain comprising the amino acid sequence shown as SEQ ID NO:1. In another embodiment, the antibody further comprises a light chain comprising the amino acid sequence selected from the group consisting of SEQ ID NO:3, SEQ ID NO:4, and SEQ ID NO:5. In one other embodiment, the antibody is an antibody which (i) neutralizes more than one subtype and/or more than one isolate of an influenza A virus, (ii) binds to a hemagglutinin (HA) antigen of the virus, and (iii) inhibits hemagglutination. In another embodiment, the antibody is an antibody which neutralizes at least one of the H1 and H3 influenza A virus subtypes. In one embodiment, the antibody is an antibody which neutralizes the H1 and H3 influenza A virus subtypes. In another embodiment, the antibody is an antibody which prevents the globular head region of the influenza A virus from binding the surface of a cell. In one other embodiment, the antibody is an antibody which prevents the influenza A virus from attaching to a cell to be infected. In another embodiment, the antibody is an antibody which binds to an H1 HA antigen. In one embodiment, the antibody is an antibody which binds to at least one additional HA antigen. In another embodiment, the additional HA antigen is H3.

In another embodiment, the antibodies or binding molecules bind to and/or are reactive to and/or neutralize more than one subtype and/or more than one isolate of an influenza A virus. In one embodiment, the virus is a virus having the ability to infect humans. In another embodiment, the isolate is an isolate that has been obtained from a human subject. In one other embodiment, the isolate is an isolate that has been obtained from a non-human animal. In another embodiment, the non-human animal is a bird. In one embodiment, the bird is a wild-fowl or a chicken. In one other embodiment, the non-human animal is a pig.

In another embodiment, the antibody or binding molecule is an antibody or binding molecule which binds to an epitope of an H1 subtype of the HA antigen. In one other embodiment, the antibody or binding molecule is an antibody or binding molecule which binds to an epitope of an H3 subtype of the HA antigen. In another embodiment, the antibody or binding molecule is an antibody or binding molecule which binds to an epitope of an H1 subtype of the HA antigen and to an epitope of an H3 subtype of the HA antigen. In one embodiment, the antibody or binding molecule is an antibody or binding molecule which binds to an epitope of an H9 subtype of the HA antigen. In another embodiment, the antibody or binding molecule is an antibody or binding molecule which binds to an epitope of an H5 subtype of the HA antigen. In another embodiment, the antibody or binding molecule is an antibody or binding molecule which binds to an epitope of an H2 subtype of the HA antigen.

In some embodiments, the antibody or binding molecule binds to an epitope which is displayed on the surface of an influenza A virus.

In some embodiments, the H1 subtype is, or the HA is from, a New Caledonia/20/99 isolate of the H1 virus; a Solomon Islands/3/06 isolate of the H1 virus; a Memphis/3/2008 isolate of the H1 virus; a Singapore/6/1986 isolate of the H1 virus; or a Beijing/262/1995 isolate of the H1 virus.

In some embodiments, the H3 subtype is, or the HA is from, a Wisconsin/67/05 isolate of the H3 virus; a Hong Kong/68 isolate of the H3 virus, a Hong Kong/1/1968 isolate of the H3 virus; a Panama/2007/1999 isolate of the H3 virus; a Moscow/10/1999 isolate of the H3 virus; a Brisbane/19/2007 isolate of the H3 virus; or a Perth/16/2009 isolate of the H1 virus.

In some embodiments, the H9 subtype is, or the HA is from, a Hong Kong/1073/99 isolate of the H9 virus; or a Turkey/Wisconsin/1/1996 isolate of the H9 virus.

In some embodiments, the H5 subtype is, or the HA is from, a Vietnam/1203/04 isolate of the H5 virus.

In some embodiments, the H2 subtype is, or the HA is from, the Adachi/1/1957 isolate of the H2 virus; a Japan/305/1957 isolate of the H1 virus; or an Adachi/2/1997 isolate of the H1 virus.

In some embodiments, the H12 subtype is, or the HA is from, a duck/Alberta/60/1976 isolate of the H12 virus.

In some embodiments, the antibody or binding molecule is an antibody or binding molecule which is cross-reactive with an H1 HA antigen and an H3 antigen. In other embodiments, the antibody or binding molecule is cross-reactive with one or more of H1, H2, H3, H5, H9, and H12.

In one aspect, the agent identified by the methods provided herein is an antibody or binding molecule which binds essentially the same epitope as the epitope for an antibody comprising a heavy chain polypeptide comprising an amino acid sequence shown as SEQ ID NO:1; or a consensus or variant sequence based upon said amino acid sequence. In another embodiment, the antibody or binding molecule binds essentially the same epitope as the epitope for an antibody comprising a light chain polypeptide comprising an amino acid sequence selected from the group consisting of SEQ ID NO:3, SEQ ID NO:4, and SEQ ID NO:5; or a consensus or variant sequence based upon said amino acid sequence. In one other embodiment, the antibody or binding molecule is an antibody or binding molecule which (i) neutralizes more than one subtype and/or more than one isolate of an influenza A virus, (ii) binds to a hemagglutinin (HA) antigen of the virus; and (iii) inhibits hemagglutination.

In another embodiment, the present invention provides a composition comprising a binding molecule or an antibody identified by the methods described herein.

In another aspect, the present invention provides methods for the treatment and/or prevention of an influenza A virus infection in a subject in need. In one embodiment, the methods comprises administering to said subject an effective amount of a composition described herein. In another embodiment, the method comprises comprising administering to said subject an effective amount of a neutralizing antibody or binding molecule described herein. In one other embodiment, the subject is a human patient.

In another aspect, the agents identified by the methods provided herein may provide the basis for designing a vaccine effective against influenza A virus infection. In one embodiment, the vaccine comprises a peptide or polypeptide functionally mimicking a neutralization epitope bound by an antibody or binding molecule described herein. In another embodiment, the vaccine is a synthetic vaccine. In one other embodiment, the vaccine comprises an attenuated influenza A virus, or a part thereof. In another embodiment, the vaccine comprises a killed influenza A virus, or part thereof. In one other embodiment, the antibody or binding molecule is selected from the group consisting of (a) an antibody or binding molecule which binds essentially the same epitope as the epitope for an antibody comprising a heavy chain polypeptide comprising an amino acid sequence shown as SEQ ID NO:1; or a consensus or variant sequence based upon said amino acid sequences; and (b) an antibody comprising a heavy chain polypeptide comprising a heavy chain polypeptide comprising an amino acid sequence shown as SEQ ID NO:1; or a consensus or variant sequence based upon said amino acid sequence.

In all embodiments, the antibody binds an HA antigen. The HA antigen may be selected from the group consisting of an H3 subtype; an H1 subtype; an H2 subtype; an H1 subtype and an H3 subtype; an H5 subtype; an H9 subtype, an H12 subtype, and any combination thereof. In one embodiment, the antigen is displayed on the surface of an influenza A virus. In another embodiment, the peptide or polypeptide functionally mimicking a neutralization epitope bound by an antibody or binding molecule described herein, comprises antigenic determinants that raise neutralizing antibodies. In another embodiment, the vaccine is suitable for oral administration; transdermal administration; or parenteral administration. In another embodiment, the vaccine is suitable for transmucosal delivery. In one other embodiment, the transmucosal delivery is intranasal administration. In another embodiment, the vaccine is for childhood immunization.

In one aspect, the agents identified by the methods provided herein are influenza neutralizing antibodies or binding molecules with a length-modified heavy chain loop. The length modified chain may be an extended heavy chain loop or a shortened heavy chain loop. In one embodiment, the antibody or binding molecule is a neutralizing antibody or binding molecule binding to a hemagglutinin of an influenza A virus having the ability to infect humans neutralizing at least one isolate of an influenza A virus, antibody having an extended heavy chain loop. In one other embodiment, the length-modified heavy chain loop is a CDR3 loop or a CDR1 loop. In another embodiment, the CDR3 loop comprises an amino acid sequence SEQ ID NO:9. In yet another antibody, the CDR1 loop comprises an amino acid sequence SEQ ID NO:7.

In another aspect, the agents identified by the methods provided herein are engineered antibodies or binding molecules with reduced oxidative potential. In one embodiment, the engineered antibody or binding molecule with reduced oxidative potential is an antibody binding to a hemagglutinin of an influenza A virus having the ability to infect humans neutralizing at least one isolate of an influenza A virus having one or more methionine substitutions in a heavy chain variable domain. In another embodiment, the heavy chain variable domain is a CDR3 region. In one other embodiment, the methionine substitution is at position 96 and/or 98 according to Kabat numbering system. In yet another embodiment, the methionine is substituted with a leucine. In one embodiment, the heavy chain variable domain includes an amino acid sequence SEQ ID NO:29.

Preferred mimetics, e.g. peptidomimetics, mimic the binding and/or biological properties of the preferred antibodies or antibody fragments herein.

Comprehensive Human Influenza Antibody Libraries

In one aspect, the agents identified by the methods provided herein originate from comprehensive human influenza antibody libraries. Comprehensive human influenza antibody libraries can be created from antibodies obtained from convalescent patients of various prior influenza, seasonal outbreaks epidemics, and pandemics, including the 1968 Hong Kong flu (H3N2), the 1957 Asian flu (H2N2), the 1918 Spanish flu (H1N1), and the 2004/2005 Avian flu (H5N1). For example, see U.S. Application Publication No. 20080014205, published on Jan. 17, 2008, incorporated herein by reference in its entirety. In order to prepare such libraries, blood or bone marrow samples are collected from individuals known or suspected to have been infected with an influenza virus. Peripheral blood samples, especially from geographically distant sources, may need to be stabilized prior to transportation and use. Kits for this purpose are well known and commercially available: such as, for example, BD Vacutainer® CPT™ cell preparation tubes can be used for centrifugal purification of lymphocytes, and guanidium, Trizol, or RNAlater used to stabilize the samples. Upon receipt of the stabilized lymphocytes or whole bone marrow, RT-PCR is performed to rescue heavy and light chain repertoires, using immunoglobulin oligo primers known in the art. The PCR repertoire products are combined with linker oligos to generate scFv libraries to clone directly in frame with m13 pIII protein, following procedures known in the art.

In a typical protocol, antibodies in the human sera can be detected by well known serological assays, including, for example, by the well-known hemagglutinin inhibition (HAI) assay (Kendal, A. P., M. S. Pereira, and J. J. Skehel. 1982. Concepts and procedures for laboratory-based influenza surveillance. U.S. Department of Health and Human Services, Public Health Service, Centers for Disease Control, Atlanta, Ga.), or the microneutralization assay (Harmon et al., J. Clin. Microbiol. 26:333-337 (1988)). This detection step might not be necessary if the serum sample has already been confirmed to contain influenza neutralizing antibodies. Lymphocytes from whole blood or those present in bone marrow are next processed by methods known in the art. Whole RNA is extracted by Tri BD reagent (Sigma) from fresh or RNAlater stabilized tissue. Subsequently, the isolated donor total RNA is further purified to mRNA using Oligotex purification (Qiagen). Next first strand cDNA synthesis, is generated by using random nonamer oligonucleotides and or oligo (dT)₁₈ primers according to the protocol of AccuScript reverse transcriptase (Stratagene). Briefly, 100 ng mRNA, 0.5 mM dNTPs and 300 ng random nonamers and or 500 ng oligo (dT)₁₈ primers in Accuscript RT buffer (Stratagene) are incubated at 65° C. for 5 min, followed by rapid cooling to 4° C. Then, 100 mM DTT, Accuscript RT, and RNAse Block are added to each reaction and incubated at 42° C. for 1 h, and the reverse transcriptase is inactivated by heating at 70° C. for 15 minutes. The cDNA obtained can be used as a template for RT-PCR amplification of the antibody heavy and light chain V genes, which can then be cloned into a vector, or, if phage display library is intended, into a phagemid vector. This procedure generates a repertoire of antibody heavy and light chain variable region clones (V_{H} and V_{L} libraries), which can be kept separate or combined for screening purposes.

Immunoglobulin repertoires from peripheral lymphocytes of survivors of earlier epidemics and pandemics, such as the 1918 Spanish Flu, can be retrieved, stabilized, and rescued in a manner similar to that described above. For additional H1 and H3 libraries repertoires can be recovered from properly timed vaccinated locally-sourced donors. As an additional option commercially available bone marrow total RNA or mRNA can be purchased from commercial sources to produce libraries suitable for H1 and H3, and depending upon the background of donor also suitable for H2 antibody screening.

Synthetic Human-Like Repertoire

In one aspect, the agents identified by the methods provided herein originate from a synthetic human antibody repertoire. In the methods of the present invention, the synthetic human antibody repertoire can be represented by a synthetic antibody library, which can be made by methods known in the art or obtained from commercial sources. Thus, for example, a fully synthetic human repertoire is described in Horowitz et al. U.S. Patent Application Publication No. 20090082213 published on Mar. 26, 2009, the entire disclosure of which is hereby expressly incorporated by reference. In brief, this patent application describes libraries of immunoglobulins in which predetermined amino acids have been combinatorially introduced into one or more complementarity-determining regions of the immunoglobulin of interest. Additionally, for example, a universal immunoglobulin library, including subsets of such library, are described in U.S. Patent Application Publication No. 20030228302 published on Dec. 11, 2003, the entire disclosure of which is hereby expressly incorporated by reference.

Specific sub-libraries of antibody heavy and light chains with various mutations can be combined to provide the framework constructs for the antibodies of the present invention, which is followed by introducing diversity in the CDRs of both heavy and light chains. This diversity can be achieved by methods known in the art, such as, for example, by Kunkel mutagenesis, and can be repeated several times in order to further increase diversity. Thus, for example, diversity into the heavy and light chain CDR1 and CD2 regions, separately or simultaneously, can be introduced by multiple rounds of Kunkel mutagenesis. If necessary, the various Kunkel clones can be segregated by CDR lengths and/or clones lacking diversity in a targeted CDR (e.g., CDR1 or CDR3) can be removed, e.g., by digestion with template-specific restriction enzymes. Upon completion of these steps, the size of the library should exceed about 10⁹ members, but libraries with lesser members are also useful.

In a specific embodiment, both immunized antibody libraries and synthetic antibody libraries are used for identifying the neutralizing antibodies of the present invention. The two types of libraries are fundamentally different. The synthetic antibody libraries are synthesized collections of human antibodies with the predicted ability to bind antigens, while an immunized repertoire will contain sequences to specifically recognize avian H5 hemagglutinin, and/or H1, H2, or H3 hemagglutinin, as the case may be. Thus, the immunized repertoires are theoretically optimized to recognize critical components of targeted influenza subtype(s). As a result these differences the two methods produce a different set of antibodies and thus provide a more efficient approach for identifying the desired neutralizing antibodies.

Hyperimmunized Non-Human Primate Antibody Libraries

In one aspect, the agents identified by the methods provided herein originate from a hyperimmunized non-human primate antibody library. In this method, an antibody library is rescued from hyperimmunized non-human primates, such as, for example, macaque or baboons. Specifically, non-human primates are immunized with various subtypes of the influenza A virus or with various hemagglutinin (H) proteins. Animals developing titers of antibody recognizing the influenza A virus subtype or hemagglutinin they were immunized with are sacrificed and their spleens harvested. Blood or bone marrow of the immunized animals is collected, and antibodies produced are collected and amplified as described above for the comprehensive influenza antibody libraries.

Strategies for Isolating Neutralizing Antibodies of the Invention

In one aspect, the agents identified by the methods provided herein utilize strategies for isolating neutralizing antibodies. Regardless of the type of antibody library or libraries used, antibodies with dual specificities, such as, for example, showing reactivity with two different influenza A subtypes and/or with two strains (isolates) of the same subtype, and/or with human and non-human isolates, can be discovered and optimized through controlled cross-reactive selection and/or directed combinatorial and/or mutagenic engineering.

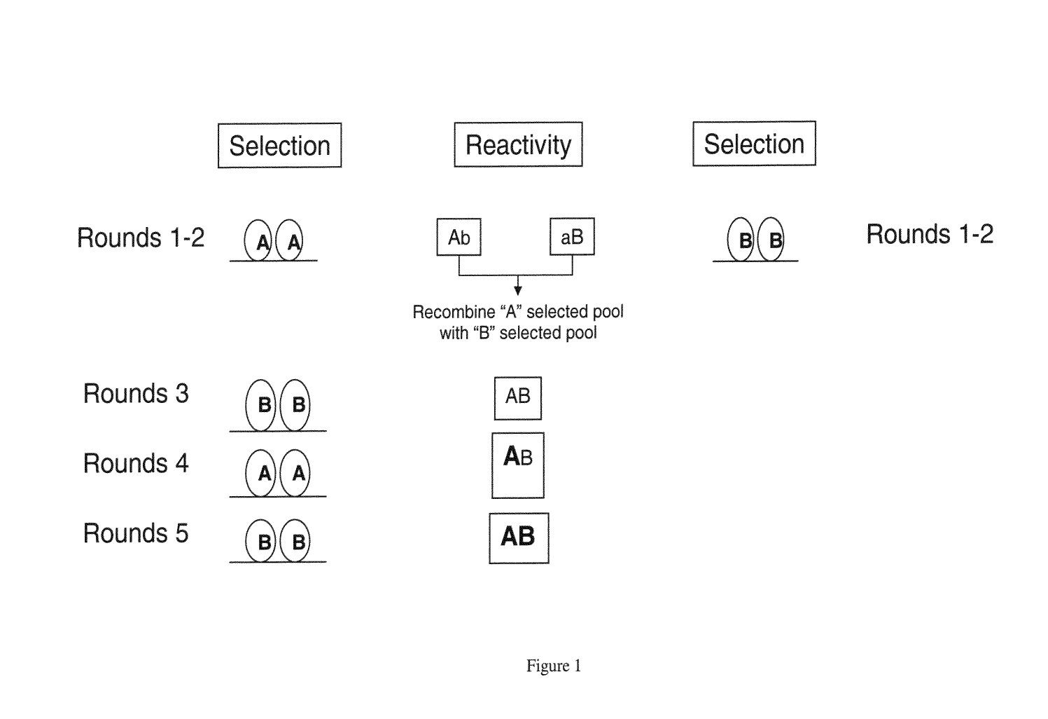

In a typical enrichment scheme, illustrated in FIG. 1, a library including antibodies showing cross-reactivity to two targets, designated as targets A and B, are subjected to multiple rounds of enrichment (see U.S. Application Publication No. 20080014205, published on Jan. 17, 2008, incorporated herein by reference in its entirety). If enrichment is based on reactivity with target A, each round of enrichment will increase the reactive strength of the pool towards target A. Similarly, if enrichment is based on reactivity with target B, each round of enrichment will increase the reactive strength of the pool towards target B. Although this approach refers to panning, which is the selection method used when screening phage display libraries (see below), the approach is equally applicable to any type of library discussed above, other otherwise known in the art, and to any type of display technique. Targets A and B include any targets to which antibodies bind, including but not limited to various isolates, types and sub-types of influenza viruses.

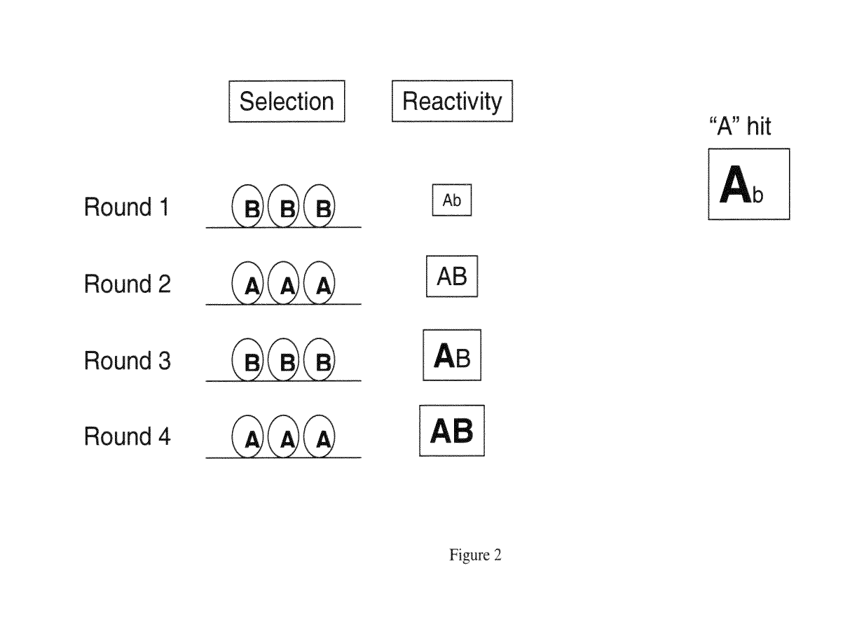

Since the goal of the present invention is to identify neutralizing antibodies with multiple specificities, a cross-reactive discovery selection scheme has been developed. In the interest of simplicity, this scheme is illustrated in FIG. 2 showing the selection of antibodies with dual specificities. In this case, an antibody library including antibodies showing reactivity with two targets, targets A and B, is first selected for reactivity with one of the targets, e.g., target A, followed by selection for reactivity with the other target, e.g., target B. Each successive selection round reinforces the reactive strength of the resulting pool towards both targets. (see also U.S. Application Publication No. 20080014205, published on Jan. 17, 2008, incorporated herein by reference in its entirety). Accordingly, this method is particularly useful for identifying antibodies with dual specificity. Of course, the method can be extended to identifying antibodies showing reactivity towards further targets, by including additional rounds of enrichment towards the additional target(s). Again, if the library screened is a phage display library, selection is performed by cross-reactive panning, but other libraries and other selection methods can also be used.

A combination of the two methods discussed above includes two separate enrichment rounds for reactivity towards target A and target B, respectively, recombining the two pools obtained, and subsequent cross-reactive selection rounds, as described above (see U.S. Application Publication No. 20080014205, published on Jan. 17, 2008, incorporated herein by reference in its entirety). This approach is illustrated in FIG. 1. Just as in the pure cross-reactive selection, each round of selection of the recombined library increases the reactive strength of the resulting pool towards both targets.

In a further embodiment, illustrated in FIG. 2, first a clone showing strong reactivity with a target A, and having detectable cross-reactivity with target B is identified. Based on this clone, a mutagenic library is prepared, which is then selected, in alternating rounds, for reactivity with target B and target A respectively. This scheme will result in antibodies that maintain strong reactivity with target A, and have increased reactivity with target B (see U.S. Application Publication No. 20080014205, published on Jan. 17, 2008, incorporated herein by reference in its entirety). Just as before, selection is performed by panning, if the libraries screened are phage display libraries, but other libraries, other display techniques, and other selection methods can also be used, following the same strategy.

As discussed above, targets A and B can, for example, be two different subtypes of the influenza A virus, two different strains (isolates) of the same influenza A virus, subtypes or isolates from two different species, where one species is preferably human. Thus, for example, target A may be an isolate of the New Caledonia isolate of the H1N1 virus, and target B may be the Wisconsin isolate of the H3N2 virus. It is emphasized that these examples are merely illustrative, and antibodies with dual and multiple specificities to any two or multiple targets can be identified, selected and optimized in an analogous manner.

Alternatively, if an antibody library such as the UAL that allows segregation of discrete frameworks and CDR lengths is used to find an antibody to target A, then an antigen B could be screened for and the library could be restricted to a diverse collection of similar parameters. Once an antibody to antigen B is found then chimeric or mutagenic antibodies based upon the respective A and B antibodies could be used to engineer a dual specific collection.

Phage Display

In a particular embodiment, the present invention utilizes phage display antibody libraries to functionally discover neutralizing monoclonal antibodies with multiple (including dual) specificities. Such antibodies can, for example, be monoclonal antibodies capable of neutralizing more than one influenza A virus subtype, including the H1, H3 and/or the H9 subtypes, the H5, H7 and/or H9 subtypes, such as the H1 and H3, H5 and H1; H5 and H2; H5 and H3; H5, H1, and H2; H5, H1, and H3; H5, H2 and H3; H1, H2 and H3, etc., subtypes, and/or more than one strain (isolate) of the same subtype.

To generate a phage antibody library, a cDNA library obtained from any source, including the libraries discussed above, is cloned into a phagemid vector.

Thus, for example, the collection of antibody heavy and light chain repertoires rescued from lymphocytes or bone marrow by RT-PCR as described above, is reassembled as a scFv library fused to m13 pIII protein. The combinatorial library will contain about more than 10⁶, or more than 10⁷, or more than 10⁸, or more than 10⁹ different members, more than 10⁷ different members or above being preferred. For quality control random clones are sequenced to assess overall repertoire complexity.

Similarly, following the initial PCR rescue of heavy and light chain variable regions from a naïve or immunized human, or hyperimmunized nonhuman primate antibody library, the PCR products are combined with linker oligos to generate scFv libraries to clone directly in frame with M13 pIII coat protein. The library will contain about more than 10⁶, or more than 10⁷, or more than 10⁸, or more than 10⁹ different members, more than 10⁷ different members or above being preferred. As a quality control step, random clones are sequenced in order to assess overall repertoire size and complexity.

Antibody phage display libraries may contain antibodies in various formats, such as in a single-chain Fv (scFv) or Fab format. For review see, e.g., Hoogenboom, Methods Mol. Biol. 178:1-37 (2002).

Screening

Screening methods for identifying antibodies and/or neutralizing agents with the desired neutralizing properties have been described above. Reactivity can be assessed based on direct binding to the desired hemagglutinin proteins.

Hemagglutinin (HA) Protein Production

Hemagglutinin (HA) proteins can be produced by recombinant DNA technology. In this method, HA genes are cloned into an appropriate vector, preferably a baculovirus expression vector for expression in baculovirus-infected insect cells, such as Spodoptera frugiperda (Sf9) cells.

The nucleic acid coding for the HA protein is inserted into a baculovirus expression vector, such as Bac-to-Bac (Invitrogen), with or without a C-terminal epitope tag, such as a poly-his (hexahistidine tag). A poly-his tag provides for easy purification by nickel chelate chromatography.

In general the cloning involves making reference cDNAs by assembly PCR from individually synthesized oligos. Corresponding isolate variant HA proteins are made by either substituting appropriate mutant oligos into additional assembly PCRs or by mutagenesis techniques, such as by Kunkel mutagenesis.

Recombinant baculovirus is generated by transfecting the above Bacmid into Sf9 cells (ATCC CRL 1711) using lipofectin (commercially available from Gibco-BRL). After 4-5 days of incubation at 28° C., the released viruses are harvested and used for further amplifications. Viral infection and protein expression are performed as described by O'Reilley et al., Baculovirus Expression Vectors: A Laboratory Manual (Oxford: Oxford University Press, 1994).

Expressed poly-His-tagged HA polypeptides can then be purified, for example, by Ni²⁺-chelate affinity chromatography as follows. Supernatents are collected from recombinant virus-infected Sf9 cells as described by Rupert et al., Nature 362:175-179 (1993). A Ni²⁺-NTA agarose column (commercially available from Qiagen) is prepared with a bed volume of 5 mL, washed with 25 mL of water, and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% glycerol, pH 6.0), which elutes non-specifically bound protein. After reaching A₂₈₀ baseline again, the column is developed with a 0 to 500 mM imidazole gradient in the secondary wash buffer. One-mL fractions are collected and analyzed by SDS-PAGE and silver staining or Western blot with Ni²⁺-NTA-conjugated to alkaline phosphatase (Qiagen). Fractions containing the eluted His₁₀-tagged HA polypeptide are pooled and dialyzed against loading buffer.

Alternatively, purification of an IgG-tagged (or Fc-tagged) HA polypeptide can be performed using known chromatography techniques, including, for instance, Protein A or protein G column chromatography.

As an alternative to using Sf9 cells HA proteins can also be produced in other recombinant host cells, prokaryote, yeast, or higher eukaryote cells. Suitable prokaryotes include but are not limited to eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. Various E. coli strains are publicly available, such as E. coli K12 strain MM294 (ATCC 31,446); E. coli X1776 (ATCC 31,537); E. coli strain W3110 (ATCC 27,325); and K5 772 (ATCC 53,635).

In addition to prokaryotes, eukaryotic microbes such as filamentous fungi or yeast are suitable cloning or expression hosts for vectors containing nucleic acid encoding an HA polypeptide. Saccharomyces cerevisiae is a commonly used lower eukaryotic host microorganism. However, a number of other genera, species, and strains are commonly available and useful herein, such as Schizosaccharomyces pombe (Beach and Nurse, Nature 290: 140 (1981); EP 139,383 published 2 May 1985); Kluyveromyces hosts (U.S. Pat. No. 4,943,529; Fleer et al., Bio/Technology 9:968-975 (1991)) such as, e.g., K. lactis (MW98-8C, CBS683, CBS4574; Louvencourt et al., J. Bacteriol. 737 (1983)), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology 8:135 (1990)), K. thermotolerans, and K. marxianus; yarrowia (EP 402,226); Pichia pastoris (EP 183,070; Sreekrishna et al., J. Basic Microbiol. 28:265-278 (1988)); Candida; Trichoderma reesia (EP 244,234); Neurospora crassa (Case et al., Proc. Natl. Acad. Sci. USA 76:5259-5263 (1979)); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun. 112:284-289 (1983); Tilburn et al., Gene 26:205-221 (1983); Yelton et al., Proc. Natl. Acad. Sci. USA 81:1470-1474 (1984)) and A. niger Kelly and Hynes, EMBO J. 4:475-479 (1985). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistry of Methylotrophs 269 (1982).

Suitable host cells for the expression of HA proteins include cells of multicellular organisms. Examples of invertebrate cells include the above-mentioned insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (HEK 293 or HEK 293 cells subcloned for growth in suspension culture (Graham et al., J. Gen Virol. 36:59 (1977)); Chinese hamster ovary cells/−DHFR (CHO, Urlaub and Chasin, Proc. Natl. Acad. Sci. USA 77:4216 (1980)); mouse sertoli cells (TM4, Mather, Biol. Reprod. 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

Hemagglutinin (HA) Protein Panning

HA protein is immobilized on to the surface of microtiter wells or magnetic beads to pan the described above libraries. In a particular embodiment, each library is allowed to bind one or more H proteins at 4 degrees for two hours and then washed extensively with cold PBS, before eluting HA specific binding clones with 0.2M glycine-HCl buffer (pH2.5). The recovered phage is pH neutralized and amplified by infecting a susceptible host E. coli. Subsequently, phagemid production can be induced to repeat the enrichment of positive clones and subsequent clones isolation for triage. Upon sufficient enrichment the entire pool is transferred by infection into a non amber suppressor E. coli strain such as HB2151 to express soluble scFv proteins. Alternatively the pool(s) could be subcloned into a monomeric scFv expression vector, such as pBAD, and recombinant soluble scFv proteins are expressed for in vitro analysis and characterization, as described below.

Characterization

Clones are tested for binding affinity to one or more H proteins, as described above. For example, binding is tested to an H1 protein (Refseq ABQ10137, Isolate New Caledonia/20/99 (H1N1) and/or Refseq ABU99069, Isolate Solomon Islands/3/06 (H1N1)), and in parallel test to an H3 protein (Refseq ACC67032, Isolate Wisconsin/67/05 (H3N2), and/or Refseq CAA24269, Hong Kong/68 (H3N2), but other isolates can also be used alone or in any combination. The positive clones obtained based on the demonstrated binding can be tested for neutralizing ability. The typical functional test for neutralization involves hemagglutination inhibition assays using whole virus binding to red blood cells. Alternatively, hemagglutination assays with recombinant hemagglutinin protein and red blood cells are possible. In order to eliminate the need for whole blood, the hemagglutinin binding inhibition assay can be performed on airway epithelial cells. The binding assay can be performed in any configuration, including, without limitation, any flow cytometric or cell ELISA (cELISA) based assays. Using cELISA is advantageous in that it obviates the use of expensive flow cytometry equipment and can provide for more automated clonal assessment and greater data collection. On the other hand, flow cytometry may provide greater sensitivity, consistency, and speed.

In one aspect, the antibodies of the present invention have a binding affinity for an H1 HA containing influenza virus and/or an H3 HA containing influenza virus. Binding affinities of the antibodies of the present invention can be determined by methods known to those of skill in the art, for example by the Scatchard analysis of Munson et al., Anal. Biochem., 107:220 (1980). In one embodiment, the binding affinity is <100 pM. In other embodiments, the binding affinity of the antibody is from about 1×10⁻⁷ to about 1×10⁻¹³ M, from about 1×10⁻⁸ to about 1×10⁻¹² M, or from about 1×10⁻⁹ to about 1×10⁻¹¹ M. In other embodiments, the binding affinity of the antibody is about 1×10⁻⁷ M, about 1×10⁻⁸ M, about 1×10⁻⁹ M, about 1×10^{-1×10} M, about 1×10⁻¹¹ M, about 1×10⁻¹² M, or about 1×10⁻¹³ M.

Optimization: Mutagenesis Libraries

For the efficient management of influenza epidemics and pandemics, including a potential pandemic associated with human infections caused by a non-human animal virus, antibodies that effectively neutralize current isolates of the H proteins, as well as future mutations, are needed. In order to achieve this goal, diverse H (e.g., H1, H3, H5, etc.) neutralizing clones need to be identified that bind all known isolates of the targeted hemagglutinin subtype(s).

Cross-reactive antibodies, in some instances, emerge through directed screening against single antigens. To increase the likelihood of isolating cross-reactivity cones one would apply multiple selective pressures by processively screening against multiple antigens. In either event cross-reactivity can be further improved by antibody optimization methods known in the art. For example, certain regions of the variable regions of the immunoglobulin chains described herein may be subjected to one or more optimization strategies, including light chain shuffling, destinational mutagenesis, CDR amalgamation, and directed mutagenesis of selected CDR and/or framework regions.

One mutagenic method designed to intentionally introduce cross-reactivity of the antibodies herein with more than one influenza A subtype and/or more than one isolate of the same subtype, is referred herein as “destinational” mutagenesis. Destinational mutagenesis can be used to rationally engineer a collection of antibodies based upon one or more antibody clones, preferably of differing reactivities. In the context of the present invention, destinational mutagenesis is used to encode single or multiple residues defined by analogous positions on like sequences such as those in the individual CDRs of antibodies. In this case, these collections are generated using oligo degeneracy to capture the range of residues found in the comparable positions. It is expected that within this collection a continuum of specificities will exist between or even beyond those of the parental clones. The objective of destinational mutagenesis is to generate diverse multifunctional antibody collections, or libraries, between two or more discrete entities or collections. In the case of influenza this method can be utilized to use two antibodies that recognize two distinct epitopes, isolates, or subtypes and morph both functional qualities into a single antibody. As an example, a first influenza A antibody can be specific to an isolate of the H1 subtype and a second antibody is specific to an isolate of the H3 subtype of the influenza A virus. To create a destinational mutagenesis library, the CDR sequences for both antibodies are first attained and aligned. Next all positions of conserved identity are fixed with a single codon to the matched residue. At non-conserved positions a degenerate codon is incorporated to encode both residues. In some instances the degenerate codon will only encode the two parental residues at this position. However, in some instances additional co-products are produced. The level of co-product production can be dialed in to force co-product production or eliminate this production dependent upon size limits or goals.

Thus, for example, if the first position of the two antibodies respectively are threonine and alanine, the degenerate codon with A/G-C- in the first two positions would only encode threonine or alanine, irrespective of the base in the third position. If, for example, the next position residues are lysine and arginine the degenerate codon A-A/G-A/G will only encode lysine or arginine. However, if the degenerate codon A/C-A/G-A/G/C/T were used then asparagine, histidine, glutamine, and serine coproducts will be generated as well.

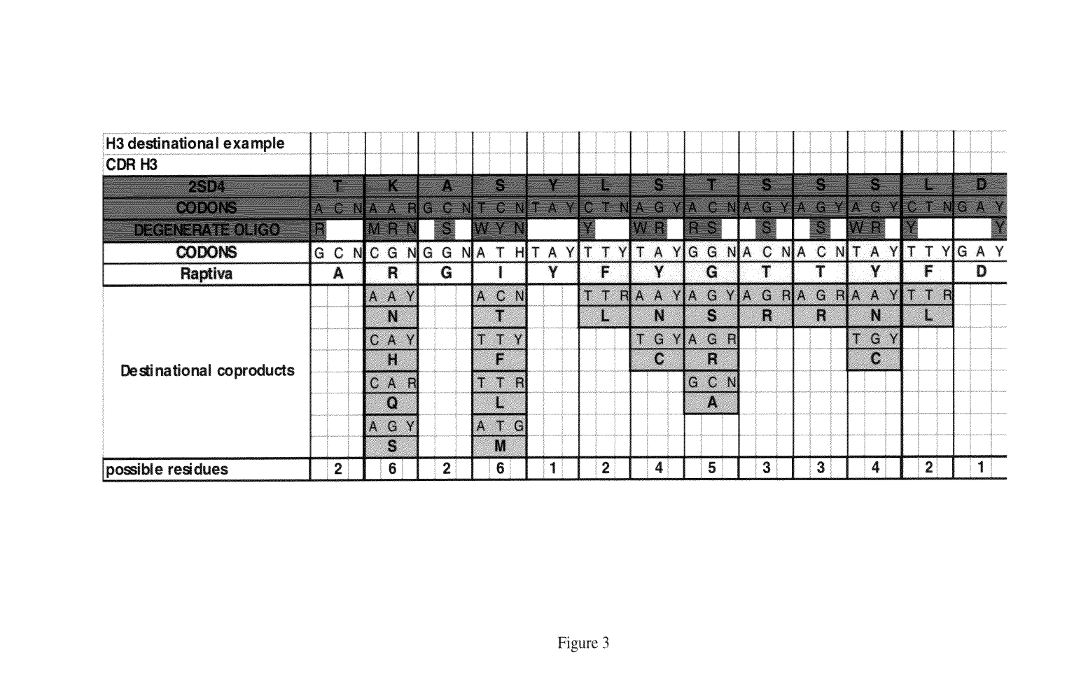

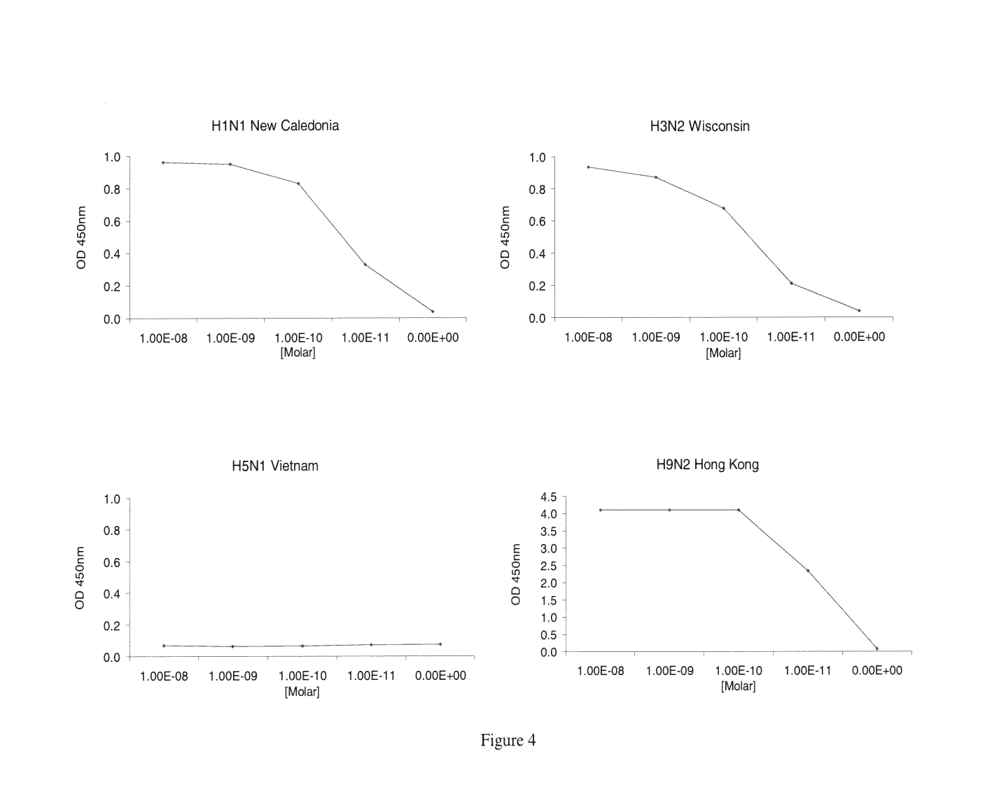

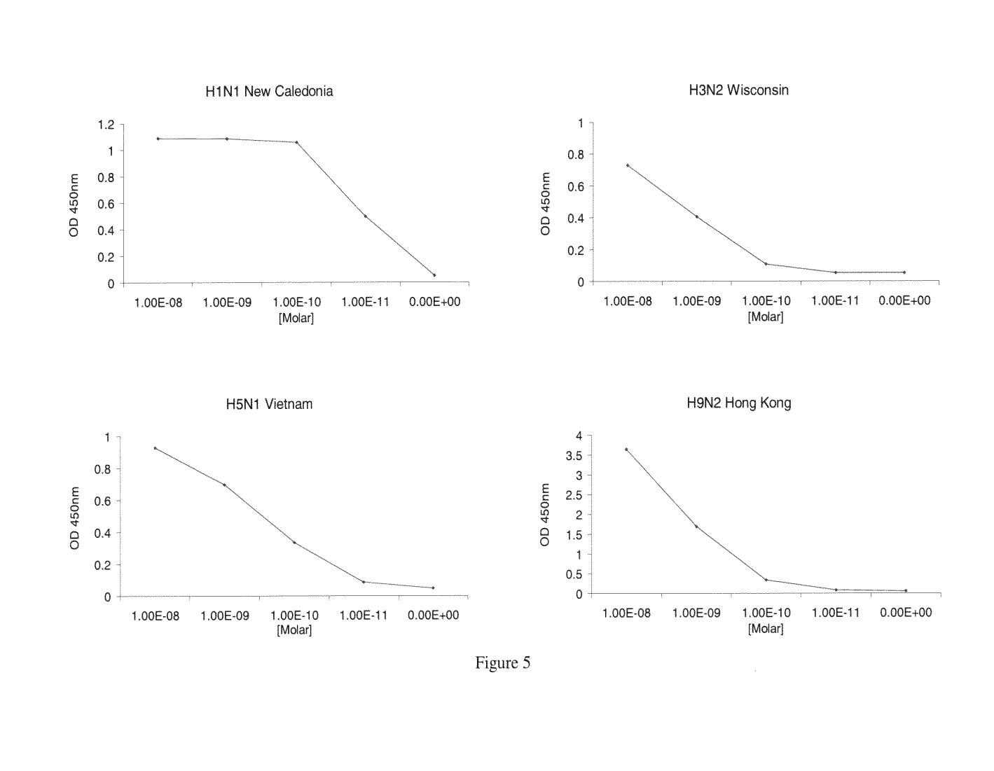

As a convenience it is simpler to use only antibodies with matched CDR lengths. One way to force this is to screen a size restricted library for the second antigen, based on the CDR length and potentially even framework restrictions imparted by the initially discovered antibody. It is noted, however, that using CDRs of equal length is only a convenience and not a requirement. It is easy to see that, while this method will be useful to create large functionally diverse libraries of influenza A virus neutralizing antibodies, its applicability is much broader. This mutagenesis technique can be used to produce functionally diverse libraries or collections of any antibody (see U.S. Application Publication No. 20080014205, published on Jan. 17, 2008 and incorporated herein by reference in its entirety). Thus, FIG. 3 is included herein to illustrate the use of the destinational mutagenesis method using CDRs of a TNF-α antibody and a CD11a antibody as the parental sequences mutagenized.

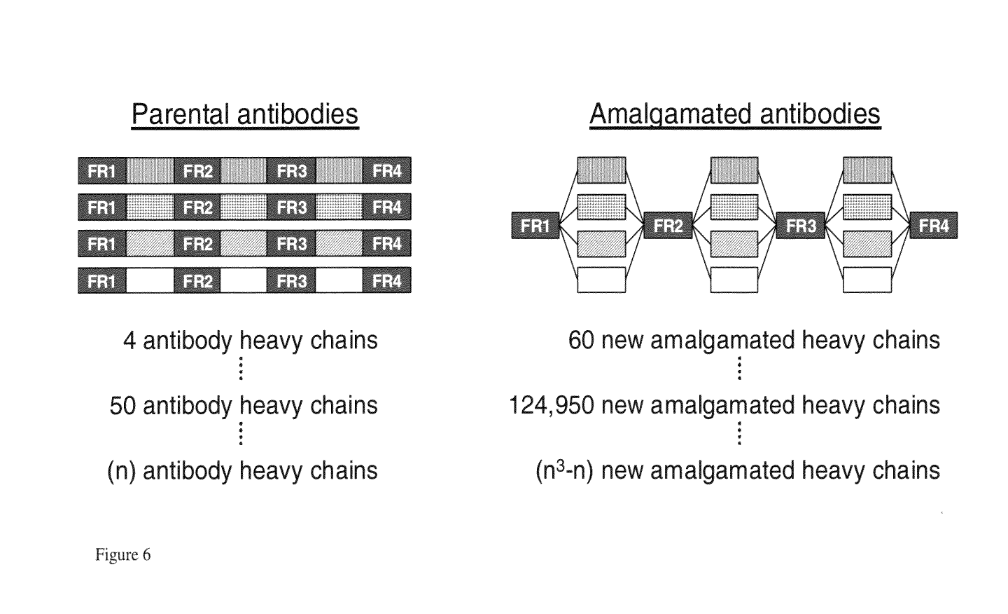

As crossreactivity is not commonly selected for naturally it is likely that executing typical mutagenic strategies may not enable potent crossreactivity. Destinational mutagenesis was devised as a directed method to generate spectrums of antibodies with crossreactive potention. Alternatively CDR amalgamation may provide another rapid and potent strategy for the creation and/or optimization of cross-reactive antibodies. It is well established that antigen binding and specificity is heavily influenced by differing combinations of selected CDRs from either or both chains. As the CDRs contained in preexisting antibodies may already be pre-optimized against target, one could create single antibodies, or collections of antibodies composed of CDR amalgams from multiple antibodies, as depicted in FIG. 6, that may prove effective against heterogeneous targets.

In FIG. 6, CDR amalgamated antibodies are depicted as a combinatorial superimposition of CDRs upon a single framework, but they could be superimposed upon multiple related and unrelated frameworks, or even chimeric frameworks thereof, increasing the overall diversity and productivity of the resulting amalgamated antibody or antibodies. Amalgamated libraries have the benefit of leveraging productive diversity found in existing antibodies, and the capacity to identify numerous new antibodies per amalgamated collection. This broader utilization of additional heavy chain frameworks allow sampling of binding in the context of CDR and framework variants that allow combinations not attainable in conventional B cell maturation from where greater crossreactivity and potency could be derivatized. As CDRs serve as interactive loops to engage target, one could create far more extensive combinations by mixing CDRs between both heavy and light chains, respective and irrespective of their original placements.

If more conventional optimization is sufficient to increase potency or spectrum of activity then targeted random mutagenesis, saturation mutagenesis, or even error-prone PCR could be utilized.

Targeted random mutagenesis (Matteuchi and Heyneker, Nucleic Acids Research 11: 3113-3121 (1983)) using ambiguously synthesized oligonucleotides is a technique that generates an intended codon as well as all possible codons at specific ratios, with respect to each other, at designated positions. Ambiguously synthesized oligonucleotides result in the reduced accuracy of nucleotide addition by the specific addition of non “wild type” bases at designated positions, or codons. This is typically performed by fixing the ratios of wild type and non wild type bases in the oligonucleotide synthesizer and designating the mixture of the two reagents at the time of synthesis.

Saturation mutagenesis (Hayashi et al., Biotechniques 17:310-315 (1994)) is a technique in which all 20 amino acids are substituted in a particular position in a protein and clones corresponding to each variant are assayed for a particular phenotype. (See, also U.S. Pat. Nos. 6,171,820; 6,358,709 and 6,361,974.)

Error prone PCR (Leung et al., Technique 1:11-15 (1989); Cadwell and Joyce, PCR Method Applic. 2:28-33 (1992)) is a modified polymerase chain reaction (PCR) technique introducing random point mutations into cloned genes. The resulting PCR products can be cloned to produce random mutant libraries or transcribed directly if a T7 promoter is incorporated within the appropriate PCR primer.

Other mutagenesis techniques are also well known and described, for example, in In Vitro Mutagenesis Protocols, J. Braman, Ed., Humana Press, 2001.

Optimization: Selection Considerations for Mutagenesis Library Screening

In the present case, one of the main goals is to engineer and isolate an antibody (or antibodies), from a collection, to effectively treat current H1 and/or H3 (or H5 or H9) isolates as well as future mutations. To engineer an antibody with tolerances capable of recognizing mutations in new isolates H1/H3, neutralizing clones that bind a variety of H1/H3 isolates need to be identified. It is expected that if a clone is selected on a first H1/H3 isolate it will bind/neutralize a second H1/H3 isolate to a lesser degree. In this case the goal is to improve recognition of the second H1/H3 isolate dramatically within the context of improving (or at least maintaining) the first H1/H3 isolate binding. Therefore, selection is first done for improvements on second H1/H3 reference protein followed by selection on the first H/H3 protein. Doing so provides a greater selective pressure on the new strain, while maintaining pressure on the second parameter.

Other H neutralizing antibodies can be optimized in an analogous manner. In this case one can select and optimize using any reference protein sequences from other isolates (e.g., H5, H9, etc.), and current as either a starting point or destination.

In addition, intertype recognition is tested with the neutralizing antibody clones. An example of intertype recognition is coincidental or engineered H1 binding from a non-H1 sourced or optimized clone.

In aggregate, the multiple mutagenesis collections and screens can be based upon the C5 and A11 antibodies, the C5-like and A11-like antibodies, C5 and A11 antibody-like molecules, C5 and C5-like surrobodies, and A11 and A11-like surrobodies. Whereby, each of the aforementioned molecules could be subject to any and all of the selections mentioned above to isolate appropriately reactive molecules that have broad spectrum reactivity and high potency.

Epitope Mapping of Neutralizing Antibodies

Once neutralizing antibodies with the desired properties have been identified, it might be desirable to identify the dominant epitope or epitopes recognized by the majority of such antibodies. Methods for epitope mapping are well known in the art and are disclosed, for example, in Morris, Glenn E., Epitope Mapping Protocols, Totowa, N.J. ed., Humana Press, 1996; and Epitope Mapping: A Practical Approach, Westwood and Hay, eds., Oxford University Press, 2001.

Epitope mapping concerns the identification of the epitope to which an antibody binds. There are many methods known to those of skill in the art for determining the location of epitopes on proteins, including crystallography analysis of the antibody-antigen complex, competition assays, gene fragment expression assays, and synthetic peptide-based assays (see for example, in Chapter 11 of Harlow and Lane, Using Antibodies, a Laboratory Manual, Cold Spring Harbor Laboratory Press, Cold Spring Harbor, N.Y., 1999; U.S. Pat. No. 7,332,579, each of which is incorporated herein by reference in its entirety). An antibody binds “essentially the same epitope” as a reference antibody, when the two antibodies recognize epitopes that are identical or sterically overlapping epitopes. A commonly used method for determining whether two antibodies bind to identical or sterically overlapping epitopes is the competition assay, which can be configured in all number of different formats, using either labeled antigen or labeled antibody. Usually, an antigen is immobilized on a 96-well plate, and the ability of unlabeled antibodies to block the binding of labeled antibodies is measured using radioactive or enzyme labels.

Production of Neutralizing Antibodies

Once antibodies with the desired neutralizing properties are identified, such antibodies, including antibody fragments can be produced by methods well known in the art, including, for example, hybridoma techniques or recombinant DNA technology.

In the hybridoma method, a mouse or other appropriate host animal, such as a hamster, is immunized to elicit lymphocytes that produce or are capable of producing antibodies that will specifically bind to the protein used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Preferred myeloma cells are those that fuse efficiently, support stable high-level production of antibody by the selected antibody-producing cells, and are sensitive to a medium such as HAT medium. Among these cell lines, preferred myeloma cell lines are murine myeloma lines, such as those derived from MOPC-21 and MPC-11 mouse tumors available from the Salk Institute Cell Distribution Center, San Diego, Calif. USA, and SP-2 or X63-Ag8-653 cells available from the American Type Culture Collection, Rockville, Md. USA. Human myeloma and mouse-human heteromyeloma cell lines also have been described for the production of human monoclonal antibodies (Kozbor, J. Immunol. 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA).

Recombinant monoclonal antibodies can, for example, be produced by isolating the DNA encoding the required antibody chains and co-transfecting a recombinant host cell with the coding sequences for co-expression, using well known recombinant expression vectors. Recombinant host cells can be prokaryotic and eukaryotic cells, such as those described above.

The choice of human variable domains, both light and heavy, to be used in making the humanized antibodies is very important to reduce antigenicity. According to the so-called “best-fit” method, the sequence of the variable domain of a rodent antibody is screened against the entire library of known human variable-domain sequences. The human sequence which is closest to that of the rodent is then accepted as the human framework region (FR) for the humanized antibody (Sims et al., J. Immunol. 151:2296 (1993); Chothia et al., J. Mol. Biol. 196:901 (1987)). It is important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences.

In addition, human antibodies can be generated following methods known in the art. For example, transgenic animals (e.g., mice) can be made that are capable, upon immunization, of producing a full repertoire of human antibodies in the absence of endogenous immunoglobulin production. See, e.g., Jakobovits et al., Proc. Natl. Acad. Sci. USA 90:2551 (1993); Jakobovits et al., Nature 362:255-258 (1993); Bruggermann et al., Year in Immuno. 7:33 (1993); and U.S. Pat. Nos. 5,591,669, 5,589,369 and 5,545,807.

Neutralizing Antibodies

In one aspect, the agents identified by the methods described herein are influenza neutralizing molecules, such as neutralizing antibodies. In one aspect, the present invention provides neutralizing antibodies that bind to a hemagglutinin protein epitope. In one embodiment, the neutralizing antibody binds to at least one epitope on the HA1 subunit of the hemagglutinin protein. In another embodiment, the neutralizing antibody binds to at least two, at least three, at least four, at least five, or at least six epitopes on the HA1 subunit of the hemagglutinin protein.

In some embodiments, the antibodies of the present invention neutralize viruses containing H3 and/or H1. In other embodiments, the antibodies neutralize both H3 and H1. In one embodiment, the antibodies of the present invention prevent hemagglutination. In other embodiments, the antibodies prevent the binding of an influenza A virus to a target cell to be infected. In another embodiment, the anti-hemagglutinin antibody prevents the receptor binding site on the globular head region of the HA of an influenza A virus from attaching to a target cell to allow hemagglutinin activity of HA to occur.

Based on the experiments described in the Examples below, a number of anti-hemagglutinin antibody heavy chain/light chain pairings were identified. In another embodiment, the antibodies of the present invention are cross-reactive to two or more influenza A virus subtypes. In one embodiment, the antibody contains a heavy chain polypeptide containing an amino acid sequence shown as SEQ ID NO:1 and a light chain polypeptide containing an amino acid sequence shown as SEQ ID NO:3, SEQ ID NO:4, or SEQ ID NO:5. In a preferred embodiment, the neutralizing antibody of the present invention binds to an epitope that is substantially the same as the epitope for (i) an antibody comprising a heavy chain amino acid sequence shown as SEQ ID NO:1 and a light chain amino acid sequence shown as SEQ ID NO:3 (clone 1286-C5 in the Example below and as shown in Table 1); (ii) an antibody comprising a heavy chain amino acid sequence shown as SEQ ID NO:1 and a light chain amino acid sequence shown as SEQ ID NO:4 (clone 1286-C5 in the Example below and as shown in Table 1); or (iii) an antibody comprising a heavy chain amino acid sequence shown as SEQ ID NO:1 and a light chain amino acid sequence shown as SEQ ID NO:5 (clone 1286-C5 in the Example below and as shown in Table 1).

TABLE 1

| | SEQ | | SEQ |
|---|---|---|---|
| Heavy Chain | ID | | ID |
| Sequence | NO | Light Chain Sequence | NO |
| QVQLQESGGGLVQPGESL | 1 | QSVLTQPPSVSGAPGQRVTISC | 3 |
| RLSCVGSGSSFGESTLSYY | | TGSSSNIGAGYDVHWYQQLP | |
| AVSWVRQAPGKGLEWLSI | | GTAPKLLIYDNNNRPSGVPDR | |
| INAGGGDIDYADSVEGRF | | FSGSKSGASASLAITGLQAEDE | |
| TISRDNSKETLYLQMTNL | | AHYYCQSYDNSLSGSVFGGGT | |
| RVEDTGVYYCAKHMSMQ | | QLTVLS | |
| QVVSAGWERADLVGDAF | | | |
| DVWGQGTMVTVSS | | | |
| | | DIQLTQSPSSLSASVGDRVTLT | 4 |
| | | CQASQDIRKFLNWYQQKPGK | |
| | | GPKLLIYDASNLQRGVPSRFSG | |
| | | GGSGTDFTLIISSLQPEDVGTY | |
| | | YCQQYDGLPFTFGGGTKLEIK | |
| | | DIQLTQSPSSLSASIGDRVTITC | 5 |
| | | QASQDIRNSLNWYEHKPGKAP | |
| | | KLLIHDASNLETGVPSRFSGGG | |
| | | SGTDFTLTISSLQPEDFATYYC | |
| | | QQANSFPLTFGGGTKVEIK | |

Antibodies with longer than typical heavy chain loops have been reported as demonstrating certain properties. For instance, a longer than typical heavy chain CDR3 loop has been linked to polyreactivity (Schettino et al. J. Immunol. 1997; 158; 2477-2489), and more recently they have been connected with numerous anti-HIV antibodies (Saphire et al. Science 2001; 293; 1151-1159; Kunert et al. AIDS Res. Hum. Retroviruses; 1998; 14(13); 1115-1128; Barbas et al. J Mol Biol 1993; 230(3):812-823) and anti-Pneumococcal antibodies (Baxendale et al. 2008; Clin. Exper. Immunol. 2007; 151; 51-60). An extended loop may facilitate deeper probing and interactions with pathogenic antigens. In the case of an HIV antibody, which contains a 19 amino acid length heavy chain CDR3, the target is engaged very specifically through the formation of a finger-like projection that contacts susceptible recessed regions on gp120. As described in Example 7, the C05 antibody heavy chain sequence has a remarkably atypical length heavy chain CDR1 loop of 11 amino acids and CDR3 loop of 25 amino acids.

In one aspect, the agent identified by the methods of the present invention is an influenza neutralizing antibody or binding molecule having a length-modified heavy chain CDR loop. Normally, the length of human heavy chain CDR 1 loop that contacts antigen is typically either about 6 or 8 amino acids and the typical length of a human heavy chain CDR3 loop that contacts antigen is about 13 amino acids (Kabat et al., Sequences of Proteins of Immunological Interest, National Institutes of Health, Bethesda, Md., ed. 5, (1991)); MacCallum et al. 1996, J. Mol. Biol. 262, 732-745). In one embodiment, the length modified heavy chain is an “extended” heavy chain CDR loop, which refers to a CDR loop that is longer than the typical heavy chain CDR loop of an antibody.

In one embodiment, the antibody or binding molecule has an extended heavy chain CDR3 loop and/or an extended heavy chain CDR1 loop. In one embodiment, the extended heavy chain CDR3 loop is about 1 amino acid, about 2 amino acids, about 3 amino acids, about 4 amino acids, about 5 amino acids, about 6 amino acids, about 7 amino acids, about 8 amino acids, about 9 amino acids, about 10 amino acids, about 11-amino acids, about 12 amino acids, about 13 amino acids, about 14 amino acids, about 15 amino acids, about 16 amino acids, about 17 amino acids, about 18 amino acids, about 19 amino acids, or about 20 amino acids longer than a typical heavy chain CDR3 loop. In another embodiment, the extended heavy chain CDR3 loop is about 1 to about 20 amino acid, about 1 to about 19 amino acids, about 1 to about 18 amino acids, about 1 to about 17 amino acids, about 1 to about 16 amino acids, about 1 to about 15 amino acids, about 1 to about 14 amino acids, about 1 to about 13 amino acids, about 1 to about 12 amino acids, about 1 to about 11 amino acids, about 1 to about 10 amino acids, about 1 to about 9 amino acids, about 1 to about 8 amino acids, about 1 to about 7 amino acids, about 1 to about 6 amino acids, about 1 to about 5 amino acids, about 1 to about 4 amino acids, about 1 to about 3 amino acids, or about 1 to about 2 amino acids longer than a typical heavy chain CDR3 loop. In yet another embodiment, the extended heavy chain CDR3 loop is about 14, about 15, about 16, about 17, about 18, about 19, about 20, about 21, about 22, about 23, about 24, about 25, about 26, about 27, about 28, about 29, about 30, about 31, about 32, about 33, about 34, or about 35 amino acids long. In a preferred embodiment, the extended heavy chain CDR3 loop is about 25 amino acids long. In another preferred embodiment, the extended heavy chain CDR3 loop comprises the extended CDR3 amino acid sequence of C05 (SEQ ED NO:31), the CDR3 sequence of C05 (SEQ ID NO:9), or a variant thereof.

As the antibodies described are capable of inhibiting hemagglutination, it is possible that this long heavy chain CDR3 loop also forms a projection that probes deeply into the globular head. Such a deep probe of the globular head may provide a novel means of interfering with sialic acid coordination in the recesses of the globular head domain of hemagglutinin. This may contribute to the remarkable breadth of activity observed for antibodies having an extended CDR3 loop, as described herein.

In one aspect, the present invention concerns potential influenza neutralizing agents capable of mimicking the binding site of an influenza virus A neutralizing molecule, e.g., a C05 antibody, which comprise amino acid sequence extensions that are discontinuous with a heavy chain hypervariable region sequence. In general, the discontinuous extension is provided by way of an amino acid modification adjacent to at least one heavy chain hypervariable region. For example, an amino acid modification may be introduced in the region that is N-terminal and/or C-terminal to the heavy chain hypervariable region. In one embodiment, the modification is an insertion of amino acids. In one other embodiment, the insertion is about 1, 2, 3, 4, 5, 6, 7, 8, 9, 10, 11, 12, 13, 14, 15, 16, 17, 18, 19, 20, 21, 22, 23, 24, or 25 amino acid residues in length. In another embodiment, the insertion is the extended CDR3 amino acid sequence of C05 (SEQ ID NO:31), the CDR3 sequence of C05 (SEQ ID NO:9), or a variant thereof. In one embodiment, the agent having the discontinuous extension is one of an antibody, antibody fragment, and surrobody. In another embodiment, the discontinuous extension allows a greater degree of contact between the CDR3 region of the agent and the HA antigen.

In one embodiment, the agent identified by the methods described herein is an antibody or binding molecule having an extended heavy chain CDR1 loop. In one embodiment, the extended heavy chain CDR1 loop is about 1 amino acid, about 2 amino acids, about 3 amino acids, about 4 amino acids, about 5 amino acids, about 6 amino acids, about 7 amino acids, about 8 amino acids, about 9 amino acids, about 10 amino acids, about 11 amino acids, about 12 amino acids, about 13 amino acids, about 14 amino acids, or about 15 amino acids longer than a typical heavy chain CDR1 loop. In another embodiment, the extended heavy chain CDR1 loop is about 1 to about 15 amino acids, about 1 to about 14 amino acids, about 1 to about 13 amino acids, about 1 to about 12 amino acids, about 1 to about 11 amino acids, about 1 to about 10 amino acids, about 1 to about 9 amino acids, about 1 to about 8 amino acids, about 1 to about 7 amino acids, about 1 to about 6 amino acids, about 1 to about 5 amino acids, about 1 to about 4 amino acids, about 1 about 3 amino acids, or about 1 to about 2 amino acids longer than a typical heavy chain CDR1 loop. In yet another embodiment, the extended heavy chain CDR3 loop is about 3, about 4, about 5, about 6, about 7, about 8, about 9, about 10, about 11, about 12, about 13, about 14, or about 15 amino acids long.

In a preferred embodiment, the extended heavy chain CDR1 loop is about 11 amino acids long. In another preferred embodiment, the extended heavy chain CDR1 loop comprises the CDR1 extended amino acid sequence (SEQ ID NO: 30), the CDR1 amino acid sequence (SEQ ID NO:7), or a variant thereof. As the heavy chain CDR1 region placement is typically proximal to CDR3 and towards the edge of the binding face, it is possible that the additional length (e.g., 3-5 amino acids) may impart additional physical docking surface area creating greater stability for the antibody to the hemaggluttinin. Curiously, analyses of the VH gene does not reveal the use of such an extended length of CDR1. Similarly, BLAST searches were unsuccessful at identifying any previously reported antibodies with such a CDR1 length. It bears consideration that positive selection played a role in reinforcing such a novel CDR1 length and composition.

In one other aspect, the length-modified heavy chain loop is a shortened heavy chain loop. The shortened heavy chain loop may comprise a deletion. In one embodiment, the CDR1 loop and/or the CDR3 loop comprise a deletion. In another embodiment, SEQ ID NO: 7 and/or SEQ ID NO:9 comprise a deletion. In one embodiment, the deletion within the heavy chain CDR1 or CDR3 loop is about 1 amino acid, about 2 amino acids, about 3 amino acids, about 4 amino acids, about 5 amino acids, about 6 amino acids, about 7 amino acids, about 8 amino acids, about 9 amino acids, about 10 amino acids, about 11 amino acids, about 12 amino acids, about 13 amino acids, about 14 amino acids, or about 15 amino acids, or about 16 amino acids, or about 16 amino acids, or about 17 amino acids, or about 18 amino acids, or about 19 amino acids, or about 20 amino acids, or about 21 amino acids, or about 22 amino acids, or about 23 amino acids, or about 24 amino acids. In another embodiment, the deletion in SEQ ID NO:7 is at least one or all of residues G-E-S-T-L (SEQ ID NO: 39-SYYAVS).

In one aspect, the agent identified by the methods described herein is an influenza neutralizing antibody or binding molecule having reduced oxidative potential or decreased oxidative heterogeneity potential. The oxidation of methionine by peroxides in aqueous formulations of polypeptides is considered to be detrimental to the development of protein-based therapeutics. The concern is at least two-fold. First, if the methionines are essential then oxidation must be controlled so that the antibody can maintain activity. However, if they are not essential for activity then non-oxidizable substitutions are preferred in order to produce antibodies or binding molecules with decreased heterogeneity. In one embodiment, the antibodies or binding molecules with reduced oxidative potential have a variant heavy chain amino acid sequence. In another embodiment, the variant amino acid sequence is in the CDR3 loop. In one embodiment, the variant amino acid sequence includes at least one substitution corresponding to an amino acid corresponding to position 96 and/or 98 of the C05 heavy chain according to Kabat numbering convention. In a preferred embodiment, a methionine at position 96 and/or 98 is the substituted amino acid. In yet another embodiment, the variant heavy chain amino acid sequence contains at least one substitution of a methionine residue. In one embodiment, the variant heavy chain amino acid sequence contains at least two substitutions of methionine residues. In a preferred embodiment, the variant sequence contains at least one methionine to leucine substitution, more preferably at least two methionine to leucine substitutions. In another embodiment, the variant sequence comprises one or more of the following:

(SEQ ID NO: 25)
AKHMSLQQVVSAGWERADLVGDAFD;
(SEQ ID NO: 26)
AKHLSMQQVVSAGWERADLVGDAFD;
(SEQ ID NO: 27)
AKHASLQQVVSAGWERADLVGDAFD;
and
(SEQ ID NO: 28)
AKHSSLQQVVSAGWERADLVGDAFD, preferably
(SEQ ID NO: 29)
AKHLSLQQVVSAGWERADLVGDAFD.

Such variant sequences may be part of a heavy chain hypervariable region sequence or it may be an amino acid sequence extension that is discontinuous with a heavy chain hypervariable region sequence, as described herein.

In one embodiment, the antibody or binding molecule with decreased oxidative heterogeneity potential retains one or more of the following features: (i) neutralizes more than one subtype and/or more than one isolate of an influenza A virus, (ii) binds to a hemagglutinin (HA) antigen of the virus, (iii) inhibits hemagglutination, or any combination thereof.

In another embodiment, variant sequences of SEQ ID NO:9 are provided including without limitation,

(SEQ ID NO: 32)
AKHMSMQQFVSAGWERADLVGDAFD
(SEQ ID NO: 33)
AKHMSMQQLVSAGWERADLVGDAFD
(SEQ ID NO: 34
AKHMSMQQVVSAGAERADLVGDAFD
(SEQ ID NO: 35)
AKHMSMQQVVSAGFERADLVGDAFD
(SEQ ID NO: 36)
AKHMSMQQVVSAGIERADLVGDAFD
(SEQ ID NO: 37)
AKHMSMAQVVSAGWERADLVGDAFD
(SEQ ID NO: 38)
AKHMSMNQVVSAGWERADLVGDAFD

In one aspect, the present invention concerns neutralizing antibodies or binding molecules with a polypeptide that comprises, consists essentially of, or consists of one or more the amino acid sequences shown as SEQ ID NOS: 1-37.

Design, Preparation and Screening of Influenza Virus Neutralizing Agents

This invention includes screening assays to identify neutralizing agents that mimic the activity of an influenza virus neutralizing antibody described herein, which find utility, for example, in the treatment and/or prevention of infection by the influenza virus.

Neutralizing agents will mimic a qualitative biological activity of the neutralizing antibody, e.g., C05. Preferably, the biological activity is the ability to inhibit the attachment or adhesion of the virus to the cell surface, e.g., by engineering an molecule, such as an influenza neutralizing mimetic, that binds directly to, or close by, the site responsible for the attachment or adhesion of the virus. Neutralization can also be achieved by a molecule directed to the virion surface, which results in the aggregation of virions. Neutralization can further occur by inhibition of the fusion of viral and cellular membranes following attachment of the virus to the target cell, by inhibition of endocytosis, inhibition of progeny virus from the infected cell, and the like. The neutralizing agents of the present invention are not limited by the mechanism by which neutralization is achieved.

In one embodiment, the influenza neutralizing agents include, for example, the immunoadhesins, peptide mimetics, and non-peptide small organic molecules mimicking a qualitative biological activity of the neutralizing antibody or a fragment thereof.

Immunoadhesins are antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

Peptide mimetics include, for example, peptides containing non-naturally occurring amino acids provided the compound retains a biological activity of a neutralizing antibody as described herein. Similarly, peptide mimetics and analogs may include non-amino acid chemical structures that mimic the structure of important structural elements of the neutralizing antibodies of the present invention and retain biological activity. The term “peptide” is used herein to refer to constrained (that is, having some element of structure as, for example, the presence of amino acids which initiate a β turn or β pleated sheet, or for example, cyclized by the presence of disulfide bonded Cys residues) or unconstrained (e.g., linear) amino acid sequences of less than about 50 amino acid residues, and preferably less than about 40 amino acids residues, including multimers, such as dimers thereof or there between. Of the peptides of less than about 40 amino acid residues, preferred are the peptides of between about 10 and about 30 amino acid residues and especially the peptides of about 20 amino acid residues. However, upon reading the instant disclosure, the skilled artisan will recognize that it is not the length of a particular peptide but its ability to bind influenza virus and neutralize it that distinguishes the peptide. Example 9 provides additional guidance on the design of peptides in relation to the present invention.

The screening and identification of neutralizing agents can be facilitated by amino acid sequence modification of the C05 antibody, and by the identification of residues that are important for the C05 antibody to bind the virus. This information can allow for the design of agents that mimic the virus binding site of the antibody. For example, it may be desirable to improve the binding affinity and/or other biological properties of an antibody. Amino acid sequence variants of the antibody may be prepared by introducing appropriate nucleotide changes into a polynucleotide that encodes the antibody, or a chain thereof, or by peptide synthesis. Such modifications include, for example, deletions from, and/or insertions into and/or substitutions of, residues within the amino acid sequences of the antibody. Any combination of deletion, insertion, and substitution may be made to arrive at the final antibody, provided that the final construct possesses the desired characteristics. The amino acid changes also may alter post-translational processes of the antibody, such as changing the number or position of glycosylation sites. Any of the variations and modifications described above for polypeptides of the present invention may be included in antibodies of the present invention.

A useful method for identification of certain residues or regions of an antibody that are preferred locations for mutagenesis is called “alanine scanning mutagenesis” as described by Cunningham and Wells in Science, 244:1081-1085 (1989). Here, a residue or group of target residues are identified (e.g., charged residues such as arg, asp, his, lys, and glu) and replaced by a neutral or negatively charged amino acid (most preferably alanine or polyalanine) to affect the interaction of the amino acids with an HA antigen. Those amino acid locations demonstrating functional sensitivity to the substitutions then are refilled by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed anti-antibody variants are screened for the desired activity.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to a cytotoxic polypeptide. Other insertional variants of an antibody include the fusion to the N- or C-terminus of the antibody to an enzyme (e.g., for ADEPT) or a polypeptide that increases the serum half-life of the antibody.

Another type of variant is an amino acid substitution variant. These variants have at least one amino acid residue in the antibody molecule replaced by a different residue. The sites of greatest interest for substitutional mutagenesis include the hypervariable regions, but FR alterations are also contemplated. Conservative and non-conservative substitutions are contemplated.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

One type of substitutional variant involves substituting one or more hypervariable region residues of a parent antibody. Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding.

Alternatively, or additionally, it may be beneficial to analyze a crystal structure of the antigen-antibody complex to identify contact points between the antibody and an antigen or infected cell. Such contact residues and neighboring residues are candidates for substitution according to the techniques elaborated herein. Once such variants are generated, the panel of variants is subjected to screening as described herein and antibodies with superior properties in one or more relevant assays may be selected for further development.

The screening and identification of neutralizing agents can be facilitated by the disclosure of the crystal structure of an antibody, e.g., C05, in complex with an influenza virus, and by the identification of the binding interface between the antibody and virus. This information can allow for the design of compounds that mimic the virus binding site of the antibody.

In addition, neutralizing agents can be identified by (a) employing computational or experimental means to perform a fitting operation between the chemical entity and the three-dimensional structure of a C05 antibody complexed with influenza virus; and (b) analyzing the data obtained in step (a) to determine the characteristics of the association between the chemical entity and the complex. Based on this information, neutralizing agent candidates can be synthesized and their binding and/or neutralizing properties can be verified in biological assays for binding and/or neutralizing activity.

In a particular embodiment, a neutralizing agent will be a chemical entity that comprises at least a portion of the influenza virus binding region of the C05 antibody, or a conservative amino acid substitution variant thereof.

In another aspect, the present invention provides methods of identifying a potential influenza virus neutralizing agent which mimics the binding site of an influenza neutralizing antibody to the influenza virus that employ three-dimensional structure information. In one embodiment, the method includes the steps of (a) employing the three-dimensional structure of an influenza neutralizing antibody bound to an influenza virus in rational drug design to design a potential influenza virus neutralizing agent which mimics the antibody binding site; and (b) contacting said potential influenza virus neutralizing agent from step (a) with an influenza virus to determine its capacity to act as a neutralizing agent. In one other embodiment, the influenza neutralizing antibody is the C05 antibody. In another embodiment, the method includes the following step prior to step (a): determining the three-dimensional structure of an influenza neutralizing antibody bound to an influenza virus. In all embodiments, the agent mimics a qualitative activity of the neutralizing antibody. In all embodiments, the agent has the ability to specifically bind influenza virus. In one embodiment, the agent is selected from the group consisting of a Surrobody, an influenza neutralizing antibody or antibody fragment, a peptide mimetic, a fusion protein, an immunoadhesin, and a small molecule.

Peptide mimetics can be conveniently prepared using solid phase peptide synthesis (Merrifield, J. Am. Chem. Soc. 85:2149 (1964); Houghten, Proc. Natl. Acad. Sci. USA 82:5132 (1985)). Solid phase synthesis begins at the carboxyl terminus of the putative peptide by coupling a protected amino acid to an inert solid support. The inert solid support can be any macromolecule capable of serving as an anchor for the C-terminus of the initial amino acid. Typically, the macromolecular support is a cross-linked polymeric resin (e.g., a polyamide or polystyrene resin). In one embodiment, the C-terminal amino acid is coupled to a polystyrene resin to form a benzyl ester. A macromolecular support is selected such that the peptide anchor link is stable under the conditions used to deprotect the \acute{α}-amino group of the blocked amino acids in peptide synthesis. If a base-labile \acute{α}-protecting group is used, then it is desirable to use an acid-labile link between the peptide and the solid support. For example, an acid-labile ether resin is effective for base-labile Fmoc-amino acid peptide synthesis. Alternatively, a peptide anchor link and \acute{α}-protecting group that are differentially labile to acidolysis can be used. For example, an aminomethyl resin such as the phenylacetamidomethyl (Pam) resin works well in conjunction with Boc-amino acid peptide synthesis. After the initial amino acid is coupled to an inert solid support, the \acute{α}-amino protecting group of the initial amino acid is removed with, for example, trifluoroacetic acid (TFA) in methylene chloride and neutralizing in, for example, triethylamine (TEA). Following deprotection of the initial amino acid's \acute{α}-amino group, the next \acute{α}-amino and side chain protected amino acid in the synthesis is added. The remaining \acute{α}-amino and, if necessary, side chain protected amino acids are then coupled sequentially in the desired order by condensation to obtain an intermediate compound connected to the solid support. Alternatively, some amino acids may be coupled to one another to form a fragment of the desired peptide followed by addition of the peptide fragment to the growing solid phase peptide chain.

The condensation reaction between two amino acids, or an amino acid and a peptide, or a peptide and a peptide can be carried out according to the usual condensation methods such as the axide method, mixed acid anhydride method, DCC (N,N′-dicyclohexylcarbodiimide) or DIC (N,N′-diisopropylcarbodiimide) methods, active ester method, p-nitrophenyl ester method, BOP (benzotriazole-1-yl-oxy-tris [dimethylamino]phosphonium hexafluorophosphate) method, N-hydroxysuccinic acid imido ester method, etc, and Woodward reagent K method.

It is common in the chemical syntheses of peptides to protect any reactive side-chain groups of the amino acids with suitable protecting groups. Ultimately, these protecting groups are removed after the desired polypeptide chain has been sequentially assembled. Also common is the protection of the \acute{α}-amino group on an amino acid or peptide fragment while the C-terminal carboxyl group of the amino acid or peptide fragment reacts with the free N-terminal amino group of the growing solid phase polypeptide chain, followed by the selective removal of the \acute{α}-amino group to permit the addition of the next amino acid or peptide fragment to the solid phase polypeptide chain. Accordingly, it is common in polypeptide synthesis that an intermediate compound is produced which contains each of the amino acid residues located in the desired sequence in the peptide chain wherein individual residues still carry side-chain protecting groups. These protecting groups can be removed substantially at the same time to produce the desired polypeptide product following removal from the solid phase: α- and ε-amino side chains can be protected with benzyloxycarbonyl (abbreviated Z), isonicotinyloxycarbonyl (iNOC), o-chlorobenzyloxycarbonyl [Z(2C1)], p-nitrobenzyloxycarbonyl [Z(NO₂)], p-methoxybenzyloxycarbonyl [Z(OMe))], t-butoxycarbonyl (Boc), t-amyloxycarbonyl (Aoc), isobornyloxycarbonyl, adamantyloxycarbonyl, 2-(4-biphenyl)-2-propyloxycarbonyl (Bpoc), 9-fluorenylmethoxycarbonyl (Fmoc), methylsulfonyethoxycarbonyl (Msc), trifluoroacetyl, phthalyl, formyl, 2-nitrophenylsulphenyl (NPS), diphenylphosphinothioyl (Ppt), and dimethylphosphinothioyl (Mpt) groups, and the like.

Protective groups for the carboxyl functional group are exemplified by benzyl ester (OBzl), cyclohexyl ester (Chx), 4-nitrobenzyl ester (ONb), t-butyl ester (Obut), 4-pyridylmethyl ester (OPic), and the like. It is often desirable that specific amino acids such as arginine, cysteine, and serine possessing a functional group other than amino and carboxyl groups are protected by a suitable protective group. For example, the guanidino group of arginine may be protected with nitro, p-toluenesulfonyl, benzyloxycarbonyl, adamantyloxycarbonyl, p-methoxybenzesulfonyl, 4-methoxy-2,6-dimethylbenzenesulfonyl (Nds), 1,3,5-trimethylphenysulfonyl (Mts), and the like. The thiol group of cysteine can be protected with p-methoxybenzyl, trityl, and the like.

Many of the blocked amino acids described above can be obtained from commercial sources such as Novabiochem (San Diego, Calif.), Bachem Calif. (Torrence, Calif.) or Peninsula Labs (Belmont, Calif.).

After the desired amino acid sequence has been completed, the peptide can be cleaved away from the solid support, recovered and purified. The peptide is removed from the solid support by a reagent capable of disrupting the peptide-solid phase link, and optionally deprotects blocked side chain functional groups on the peptide. In one embodiment, the peptide is cleaved away from the solid phase by acidolysis with liquid hydrofluoric acid (HF), which also removes any remaining side chain protective groups. Preferably, in order to avoid alkylation of residues in the peptide (for example, alkylation of methionine, cysteine, and tyrosine residues), the acidolysis reaction mixture contains thio-cresol and cresol scavengers. Following HF cleavage, the resin is washed with ether, and the free peptide is extracted from the solid phase with sequential washes of acetic acid solutions. The combined washes are lyophilized, and the peptide is purified.

In one embodiment, a group of influenza neutralizing agents includes antibodies specifically binding influenza virus and inhibiting the biological activity of the virus.

In one embodiment, the influenza neutralizing agents include, for example, antibodies that mimic a qualitative biological activity of the C05 neutralizing antibody or a fragment thereof. Exemplary antibodies (both agonists and antagonists) include polyclonal, monoclonal, humanized, bispecific and heteroconjugate antibodies, and antibody fragments.

Antibodies which recognize and bind to the influenza virus or which act to neutralize the virus may, alternatively be monoclonal antibodies. Monoclonal antibodies may be prepared using hybridoma methods, such as those described in the art (see Kohler and Milstein, Nature, 256:495 (1975); Goding, Monoclonal Antibodies: Principles and Practice, Academic Press, (1986) pp. 59-103; Kozbor, J. Immunol., 133:3001 (1984); Brodeur et al., Monoclonal Antibody Production Techniques and Applications, Marcel Dekker, Inc., New York, (1987) pp. 51-63). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include an influenza virus polypeptide, an antigenic fragment or a fusion protein thereof. In a preferred embodiment, the immunizing agent will be designed based up knowledge of the interaction between an influenza neutralizing antibody, e.g., C05, and the virus.

The monoclonal antibodies may also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA may be placed into expression vectors, which are then transfected into host cells such as simian COS cells, Chinese hamster ovary (CHO) cells, or myeloma cells that do not otherwise produce immunoglobulin protein, to obtain the synthesis of monoclonal. antibodies in the recombinant host cells. The DNA also may be modified, for example, by substituting the coding sequence for human heavy and light chain constant domains in place of the homologous murine sequences [U.S. Pat. No. 4,816,567; Morrison et al., supra] or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies are preferably monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

The antibodies of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. Humanization can be essentially performed following the method of Winter and coworkers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985); Boerner et al., J. Immunol., 147(1):86-95 (1991); U.S. Pat. No. 5,750,373]. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature.sub.—Biotechnology 14, 845-51 (1996); Neuberger, Nature Biotechnology 14, 826 (1996); Lonberg and Huszar, Intern. Rev. Immunol. 13 65-93 (1995).

Bispecific antibodies are monoclonal, preferably human or humanized, antibodies that have binding specificities for at least two different antigens. In the present case, one of the binding specificities may be for the polypeptide of the invention, the other one is for any other antigen, and preferably for a cell-surface protein or receptor or receptor subunit.

Methods for making bispecific antibodies are known in the art. Traditionally, the recombinant production of bispecific antibodies is based on the coexpression of two immunoglobulin heavy-chain/light-chain pairs, where the two heavy chains have different specificities (Milstein and Cuello, Nature, 305:537-539 [1983]). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are cotransfected into a suitable host organism. For further details of generating bispecific antibodies see, for example, Suresh et al., Methods in Enzymology, 121:210 (1986).

Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

It may be desirable to modify the antibody of the invention with respect to effector function, so as to enhance the effectiveness of the antibody in treating an immune related disease, for example. For example cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B., J. Immunol. 148:2918-2922 (1992). Homodimeric antibodies with enhanced anti-tumor activity may also be prepared using heterobifunctional cross-linkers as described in Wolff et al. Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design, 3:219-230 (1989).

The influenza neutralizing agents of the present invention can be tested in a variety of in vitro and in vivo assays to determine whether they mimic or inhibit a biological activity of the influenza neutralizing antibody, e.g., the C05 antibody.

As a result of their ability to neutralize influenza virus, the neutralizing agents find utility in the prevention and/or treatment of influenza virus infection.

Influenza Neutralizing Mimetics

Influenza neutralizing mimetics may be derived from and/or designed based upon an influenza neutralizing molecule described herein, e.g., C05. The mimetics include, but are not limited to: peptide mimetics including peptides, proteins, and derivatives thereof, such as peptides containing naturally occurring, non-naturally occurring, or non-peptide organic moieties, synthetic peptides which may or may not contain amino acids and/or peptide bonds, but retain the structural and functional features of a peptide ligand. Influenza neutralizing mimetics may also be based upon fragments of an influenza neutralizing antibody, such as digestion fragments, specified portions and variants thereof, including, without limitation, antibody mimetics or comprising portions of antibodies that mimic the structure and/or function of an antibody or specified fragment or portion thereof, including, without limitation, single chain antibodies, single domain antibodies, minibodies, and fragments thereof. Functional fragments include antigen-binding fragments that bind to an influenza virus antigen of interest.

For example, antibody fragments capable of binding to a target antigen or portions thereof, including, but not limited to, Fab (e.g., by papain digestion), Fab′ (e.g., by pepsin digestion and partial reduction) and F(ab′).sub.2 (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the term antibody (see, e.g., Colligan, et al., eds., Current Protocols in Immunology, John Wiley & Sons, Inc., NY (1994-2006). Thus, the term antibody, as used herein also includes antibody fragments either produced by the modification of whole antibodies or synthesized de novo using recombinant DNA methodologies.

Recombinant and Chemical Synthesis of C05 CDR Peptides and Analogs

Functional neutralization of influenza virus infection by an influenza neutralizing molecule described herein (e.g., the C05 antibody) is mediated through heavy chain complementarity determining region (CDR) 3 (CDRH3 hereafter) by its occupation of the receptor binding site on the influenza hemagglutinin protein. Specific points of contact between amino acids in the CDRH3 and the hemagglutinin protein are required binding and to block binding to sialic acids on the surface of susceptible cells. As the heavy chain CDR (1) (CDRH1 hereinafter) region placement is typically proximal to CDR3 and towards the edge of the binding face, it is possible that the additional length (e.g., 3-5 amino acids) of CDR1H1 may impart additional physical docking surface area creating greater stability for the antibody to the hemaggluttinin.

Peptides comprised of the CDRH3 and/or CDRH1 amino acid sequence and variants thereof can be made for use in blocking the hemagglutinin receptor binding site. As antibody CDRs are amino acid loops that extend from immunoglobulin scaffolds, free peptides (untethered and unconstrained) may be made that mimic their effect on the hemagglutinin protein. Such peptides may be made from the 20 naturally occurring amino acids through recombinant expression in a suitable expression system such as bacteria, fungi, insect and mammalian cells. To achieve this, DNA encoding the CDRH3, CDRH1 and/or variants thereof is cloned into a plasmid vector compatible with the expression system. Amino acid changes can be made at one or multiple positions using site directed mutagenesis techniques such as strand overlap exchange polymerase chain reaction (SOE-PCR) or the incorporation of oligonucleotides via the method of Kunkel. Alternatively, the entire CDR peptide and/or variants can be synthesized as oligonucleotides with flanking restriction enzyme sites for use in sub-cloning techniques known in the art.

For ease of purification, CDR peptides and variants may be made as fusion proteins to molecules known in the art to aid in expression, solubility and purification. For example poorly soluble peptides can be made more soluble through fusion to a highly soluble protein. Removal of the fusion protein by a proteolytic enzyme such as Thrombin, Factor Xa, Trypsin, and others, can be performed to release a free peptide once the fusion partner is immobilized by liquid chromatography affinity capture. In addition, specific variants of such expression systems that have been engineered to allow the incorporation of unnatural amino acids may also be used to expand the types of recombinant peptide variants.

Alternative to recombinant methods, chemical synthesis methods to incorporate natural and unnatural amino acids and their analogs may be used to create CDR peptides. Chemical synthesis offers the advantage of incorporating multiple and different unnatural amino acids into the CDR peptide sequence during a single synthesis. Recombinant methods are limited by the availability of aminoacyl tRNA synthetases that can be engineered (typically 1) to accept unnatural amino acids and the organisms which are amenable to such modifications. Moreover, the efficiency of synthetic methods is greater than recombinant methods in incorporating unnatural amino acids.

Constrained Heavy Chain CDR Peptides

As the heavy chain CDRH3 and CDRH1 loops are presented within the context of a heavy chain immunoglobulin framework where both termini are attached to, and constrained by the framework structure it could be beneficial to constrain the termini of the heavy chain CDRH1 and/or CDRH3 peptides with chemical and/or biochemical conjugates to reconstitute the necessary and favored interactive structures typically supported by the immunoglobulin framework. One strategy to generate such peptide loops would be to synthesize the heavy chain CDRH3 flanked by cysteine residues, whereby selective intrapeptide cysteine disulfide bonding could be used to generate peptide loops similar to those found in the native antibody structure. In creating such cysteine-flanked peptides it is possible that positioning the cysteines to immediately flank the CDRH3 peptide sequence might restrict the optimal loop structure from forming. In this case amino acid, or non-amino acid linkers, could be used as intervening spacers between the peptide and the cysteines.

As an alternative to disulfide bridging, other chemistries could provide specificity and loop structure such as amine-reactive bifunctional linkers. It would be possible to incorporate amine reactive sites to flank the CDRH1 and/or CDRH3 peptide by incorporating primary amines through lysines or other specific conjugates at the carboxy terminus and then use any number of amine reactive bifunctional agents to form intrapeptide bonds via a amine terminal and carboxy terminal amine.

Moreover, heterobifunctional linkers utilizing differing chemistries could be specifically programmed to match defined chemistries incorporated at specific ends of the peptides, increasing the production of the desired peptide loop.

Finally, the peptides could also be synthesized to contain biotin at both amino and carboxy termini. These biotin containing peptides could be combined with avidin, or streptavidin to bind the peptides via the biotins in a manner that favors intramolecular binding to create peptide loops.

Constrained Heavy Chain CDR Peptides Through Protein Scaffold Constraints

As the heavy chain CDRH3 and CDRH1 loops are presented within the context of a heavy chain immunoglobulin framework where both termini are attached to, and constrained by the framework structure it may be beneficial to constrain the termini of the heavy chain CDRH1 and/or CDRH3 peptides with a surrogate protein scaffold to reconstitute the necessary and favored interactive structures typically supported by the immunoglobulin variable domain frameworks. Structured protein loops are not restricted to the variable domains of antibodies. Constant regions, such as those found in the Fc domain, can accommodate substitutions in exterior loops. Alternatively, it is possible to graft the CDRH1 and/or CDRH3 loops into other immune-based protein scaffolds, such as CTLA-IV or into nonimmune-based protein scaffolds, such as Fibronectin domains.

Antibody Hybrids Through CDR Peptide Replacement of Existing CDRs and Inclusion into Non-CDR Domain Loops

Antibodies are heterotetrameric complexes composed of two heavy chain and two light chain subunits. Within each subunit are three hypervariable regions or complementarity determining regions (CDRs) that are presented peptide loops from the antibody framework. These regions are the major determinants of antigen recognition and high affinity binding.

Because of the independent nature of the loops in the immunoglobulin framework, it is possible to replace CDR loops in one antibody with those from a different antibody(s) to impart unique properties without causing major alterations to the immunoglobulin structure. Additionally, the incorporation of a CDRH1 and/or CDRH3 from the same or a different antibody into more than one CDR either in the heavy or light chain can also provide avid binding and/or new function to an antibody. An alternative strategy to bring new properties to an antibody is to add a CDRH1 and/or CDRH3, whole or in part, from an antibody of one function to a CDR, whole or in part, in a second antibody with different antigen recognition to create a hybrid CDR having functions from both.

Antibody structures contain regions outside of the CDRs that may also accommodate peptide loops to impart additional functionality. The constant domain of antibodies are highly conserved and participate in important binding interactions that mediate the effector functions and FcRn binding for long lasting pharmacokinetic properties. Loops in the CH2 and CH3 domains can be replaced with functional peptides taken from CDRs. Non-CDR loops in the variable domains may also be replaced with CDR loops from the same or other antibodies to create additional hybrid antibodies. An additional benefit of this approach is to impart humanization of CDRs from non-human species when hybridized on a human antibody.

Generating Variants with Increased Affinity and Breadth of Activity

Various methods of mutagenesis are used to create improved variants for testing either individually or amongst a collection in a library. Methods commonly used to introduce beneficial mutations at sites responsible for binding, such as the CDRs or those contact residues found specifically through direct structural analysis would be, but not limited to, saturation mutagenesis, Look through mutagenesis, or parsimonious mutagenesis.

As a directed step one would create a collection of variants based upon mutagenesis of the CDR3 and/or CDR1 by the methods mentioned above. Previous work with other human antibodies have shown tremendous benefits by exploring and generating point variants to the light chain, other heavy chain CDRs, or even simultaneously to several or all of these areas at one time to produce synergistic improvements. Usually one can accomplish this by maintaining the parental framework and length of the CDRs, while varying only the composition of the CDRs. Conversely error-prone PCR mutagenesis and other stochastic processes could be used throughout similar regions and also in other areas of the heavy chain variable domain to generate collections of variants. In any event the resulting collections or clones could be selected for increased affinity, neutralization, and or breadth of activity.

As described above we would generate such optimization collections, but because the heavy chain is marked by such unique loop lengths in both CDR1 and CDR3 it could be of even greater importance to test the potency and breadth of activity not just by varying the composition of these loops, but also by varying the length of these loops. For example, we would test the effects of the extended CDR1 loop by replacement with the corresponding shorter germline CDR1 peptide sequence and/or a mutated collection. In addition insertion of random amino acids within CDR1, or at the FR1 and FR2 junctions of CDR1 in a stepwise library fashion, until the loop matches, and even exceeds the existing extended loop length could be made and discriminately screened for better and broader binders. Similarly the CDR3 sequences could be contracted or expanded within the loops or at the FR3 and FR4 junctions of CDR3 in a step wise library fashion. By analogy to other mutually beneficial mutagenesis the varied loop length CDR1 and CDR3 libraries could be combined to interrogate novel and more broadly potent anti-influenza antibodies.

Use of Neutralizing Antibodies and Agents

In one aspect, the agents identified by the methods described herein are influenza neutralizing molecules, e.g., antibodies, antibody-like molecules and neutralizing agents, that can be used for the prevention and/or treatment of influenza type A infections and for the development of vaccines presenting the appropriate cross neutralizing epitopes. For therapeutic applications, the molecules are usually used in the form of pharmaceutical compositions. Techniques and formulations generally may be found in Remington: The Science and Practice of Pharmacy, 21^{st} Edition, Lippincott Williams & Wilkins (2005). See also, Wang and Hanson “Parenteral Formulations of Proteins and Peptides: Stability and Stabilizers,” Journal of Parenteral Science and Technology, Technical Report No. 10, Supp. 42-2S (1988).

In one aspect, the agents identified by the methods described herein are suitable for use in methods of treating or preventing influenza in a subject in need. In one embodiment, the method includes the step of administering an agent to a subject in need. The agent may be an influenza neutralizing molecule and similar to the C05 antibody (see Example 6), the agent may provide a therapeutic or prophylactic effect against influenza infection.

Agents identified by the methods described herein, such as antibodies, are typically formulated in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The agents also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, supra. Agents identified by the methods described herein may also be formulated as immunoliposomes. Liposomes containing the agent, e.g., an antibody, are prepared by methods known in the art, such as described in Epstein et al., Proc. Natl. Acad. Sci. USA 82:3688 (1985); Hwang et al., Proc. Natl. Acad. Sci. USA 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al. J. Biol. Chem. 257:286-288 (1982) via a disulfide interchange reaction. A chemotherapeutic agent is optionally contained within the liposome. See Gabizon et al. J. National Cancer Inst. 81(19)1484 (1989).

For the prevention or treatment of disease, the appropriate dosage of an agent, e.g., an antibody, will depend on the type of infection to be treated the severity and course of the disease, and whether the antibody is administered for preventive or therapeutic purposes. The agent is suitably administered to the patient at one time or over a series of treatments. Depending on the type and severity of the disease, about 1 μg/kg to about 15 mg/kg of the agent, e.g., an antibody, is a typical initial candidate dosage for administration to the patient, whether, for example, by one or more separate administrations, or by continuous infusion. In other embodiments, the dosage is about 0.010 mg/kg, about 0.015 mg/kg, about 0.020 mg/kg 0.025 mg/kg, about 0.030 mg/kg, about 0.035 mg/kg, about 0.040 mg/kg, about 0.045 mg/kg, about 0.050 mg/kg, about 0.055 mg/kg, about 0.060 mg/kg, about 0.065 mg/kg, about 0.070 mg/kg, about 0.075 mg/kg, about 0.080 mg/kg, about 0.085 mg/kg, about 0.090 mg/kg, about 0.10 mg/kg, about 0.15 mg/kg, about 0.20 mg/kg, about 0.25 mg/kg, about 0.30 mg/kg, about 0.35 mg/kg, about 0.40 mg/kg, about 0.45 mg/kg, about 0.50 mg/kg, about 0.55 mg/kg, about 0.60 mg/kg, about 0.65 mg/kg, about 0.70 mg/kg, about 0.75 mg/kg, about 0.80 mg/kg, about 0.85 mg/kg, about 0.90 mg/kg, about 0.95 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg.

The agents identified by the methods of the present invention can be additionally used as a tool for epitope mapping of antigenic determinants of an influenza A virus, and are useful in vaccine development. Indeed, as shown in the Example below, broadly reactive neutralizing antibodies can be used as guides for vaccine design.

Thus, the agents, e.g., antibodies, identified by the methods of the present invention can be used to select peptides or polypeptides that functionally mimic the neutralization epitopes to which the antibodies bind, which, in turn, can be developed into vaccines against influenza A virus infection. In one embodiment, the present invention provides a vaccine effective against an influenza A virus comprising a peptide or polypeptide that functionally mimics a neutralization epitope bound by an antibody described herein. In one embodiment, the vaccine comprises a peptide or polypeptide functionally mimicking a neutralization epitope bound by an antibody that binds a hemagglutinin (HA) antigen. In another embodiment, the vaccine may be synthetic. In other embodiments, the vaccine may comprise (i) an attenuated influenza A virus, or a part thereof; or (ii) a killed influenza A virus, or part thereof. In one other embodiment, the vaccine comprises a peptide or polypeptide functionally mimicking a neutralization epitope bound by an antibody that binds a hemagglutinin (HA) antigen. The HA antigen may be an H3 subtype or an H1 subtype. In another embodiment, the HA antigen is displayed on the surface of an influenza A virus.

In another embodiment, the peptides or polypeptides of the vaccine contain antigenic determinants that raise crossreactive influenza A virus neutralizing antibodies.

In one aspect, the present invention provides the use of the agents described herein for the preparation of a medicament or pharmaceutical composition useful in or for the prevention or treatment of a disease in a subject in need. In another embodiment, the present invention provides pharmaceutical compositions for treating or preventing a disease in a subject in need, said composition comprising an agent, e.g., an influenza neutralizing molecule, described herein.

Non-Antibody Molecules with Neutralizing Properties

Although in the previous description the invention is illustrated with reference to antibody libraries, libraries of other, non-antibody molecules, such as surrobodies, can be prepared, used, and optimized in a similar manner. Thus, the construction of unique combinatorial protein libraries based on the pre-B cell receptor (pre-BCR) (“surrobody libraries”) are described in Xu et al., 2008, supra. As discussed before, the pre-BCR is a protein that is produced during normal development of the antibody repertoire. Unlike that of canonical antibodies, the pre-BCR subunit is a trimer that is composed of an antibody heavy chain paired with two surrogate light chain (SLC) components. Combinatorial libraries based on these pre-BCR proteins in which diverse heavy chains are paired with a fixed SLC were expressed in mammalian, Escherichia coli, and phagemid systems. These libraries contain members that have nanomolar affinity for a target antigen. A description of the library construction, selective enrichment, and biophysical characterization of library members is detailed in the Materials and Methods section of Xu et al., (2008), supra. Any of the antibody sequences described herein may be used to construct such binding or non-antibody molecules, such as for example surrobodies.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. Thus, for an embodiment of the invention using one of the terms, the invention also includes another embodiment wherein one of these terms is replaced with another of these terms. In each embodiment, the terms have their established meaning. Thus, for example, one embodiment may encompass, a molecule “comprising” a number of components, another embodiment would encompass a molecule “consisting essentially of” the same components, and a third embodiment would encompass a molecule “consisting of” the same components. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The foregoing written description is considered to be sufficient to enable one skilled in the art to practice the invention. The following Examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

All patents, patent applications, and literature references cited in the present specification are hereby incorporated by reference in their entirety.

### Example 1

### Antibody Libraries from Influenza Donors

Donors selected for inclusion into the Comprehensive Influenza Library were confirmed to have had a previous influenza infection, been approximately 5 years old at the time of a the 1957 H2N2 or 1968 H3N2 influenza pandemics, and to be in current good health. Serology on a panel of H1N1 A/NewCaledonia/20/99, H3N2 A/Panama/2007/99 and H5N1 A/Vietnam/1203/2004 virus or hemagglutinin proteins was performed to confirm the presence of antibodies to the hemagglutinin proteins.

First 5-20 ml of bone marrow was collected from each donor meeting the selection criteria and mixed with RNAlater (Applied Biosystems) per the manufacturer's instructions to preserve the integrity of cellular RNA. RNA was isolated using a TRI-BD reagent protocol (Sigma-Aldrich).

Heavy chain and light chain repertoires were recovered from each donor derived RNA by RT-PCR using random primed cDNA template for heavy chains, oligo dT primed cDNA template for light chains and gene specific variable domain primers.

Next, 1 μg each of pooled Kappa light chain and pooled Lambda light chain per donor are digested with NotI and BamHI and gel purified using Qiagen Gel Extraction Kit. For kappa and lambda light chain cloning 5 μg of each vector was digested with NotI and BamHI and gel purified using Qiagen Gel Extraction Kit. Light chain library ligations are performed with 200 ng of gel purified Kappa or Lambda inserts and 1 μg of gel purified vector. Incubation is overnight at 14° C. To determine cloning efficiencies, a control ligation reaction is set up equal to the amount of one electroporation (200-300 ng vector DNA) without the addition of light chain inserts. Prior to transformation the ligations are desalted using Edge BioSystem Perfroma spin columns. Each library was transformed in 3-5 electroporations using 80 μl Dh5α electrocompentent cell aliquots, with each recovered into 1 ml SOC, pooled and outgrown for one hour at 37 C. A sample of each library is plated on selective media and used to determine the efficiency of cloning and total number of transformants. The remainder is transferred to 200 ml 2YT+100 ug/ml Ampicillin+2% glucose and grown overnight at 37° C. Successful libraries have background of less than 10% and total transformants exceeding 1×10⁶ members. The following day light chain library plasmids were isolated using a Qiagen High Speed Maxiprep Kit.

To clone heavy chain collections 1.5-2 μg each of the 5 donor specific heavy chains variable genes (VH1/7, VH 2, 5, 6, VH 3, and VH 4) are digested with a 40 Unit excess/ug DNA with SfiI and XhoI and gel purified using Qiagen Gel Extraction Kit. To prepare the recipient plasmid 15 μg of each light chain library vector is digested with a 40 Unit excess/ug DNA with SfiI and XhoI and gel purified using Qiagen Gel Extraction Kit. Library ligations are accomplished by combining 1.2 μg SfiI/XhoI digested, gel purified heavy chain DNA per donor pooled to contain 300 ng of each of the 5 heavy chain variable gene families with 5 μg of each light chain library, Kappa and Lambda respectively. A control ligation reaction is set up equal to the amount of one electroporation. (300-600 ng vector DNA) without the addition of heavy chain inserts. The ligations were incubated overnight at 14° C. and then desalted with Edge BioSystem Pefroma spin columns. The ligation was transformed into 8-12 electroporations per library are done using 80 ul TG-1 cells, each recovered into 1 ml SOC, pooled and outgrown for one hour at 37° C. A sample of each was used to determine the efficiency of cloning and the total number of transformants. Target number of transformants/library should be at least 1×10⁷ with a background of less than 10%. The remainder was transferred to 300 ml 2YT+100 ug/ml Ampicillin+2% glucose and grown to an OD600 of −0.3. Next m13K07 helper phage was added at a multiplicity of infection (MOI) of 5:1 and incubated for 1 hour at 37° C. without shaking. Following helper infection, the cells were harvested by centrifugation and resuspended in 300 ml 2YT+100 ug/ml Ampicillin+2% glucose+70 ug/ml Kanamycin and growth continued at 37° C. overnight with shaking for stock phage production.

The resulting phage containing culture supernatents are harvested by centrifugation at 6000 RPM for 10 minutes at 4° C. Next the phage are precipitated by the addition of 0.2 volume of 20% PEG/2.5M NaCl solution to each supernatant and incubation on ice for 1 hour. Phage are then harvested by centrifugation at 7900 RPM for 15 minutes at 4 C. The supernatant is removed and the phage pellet resuspended in 30 ml sterile 1×PBS. For long term −20° C. storage the PBS is supplemented with 50% glycerol.

### Example 2

### Preparation of Neutralizing Antibodies

Antibodies derived from human bone marrow phage display antibody libraries (see Example 1) were converted and tested as mammalian expressed immunoglobulins, as previously described (see also, Kashyap A K et al., Proc Natl Acad Sci USA. 2008 Apr. 22; 105(16):5986-91). The heavy chain sequence of the 1286-C5 is provided below:

(SEQ ID NO: 1)
QVQLQESGGGLVQPGESLRLSCVGSGSSFGESTLSYYAVSWVRQAPGKGLEWLSIINAG
GGDIDYADSVEGRFTISRDNSKETLYLQMTNLRVEDTGVYYCAKHMSMQQVVSAGWE
RADLVGDAFDVWGQGTMVTVSS

The underlined hypervariable CDR regions are shown for the heavy chains as follows.

(SEQ ID NO: 7)
GESTLSYYAVS
(SEQ ID NO: 8)
WLSIINAGGGDID
(SEQ ID NO: 9)
AKHMSMQQVVSAGWERADLVGDAFD

The heavy chain described by SEQ ID NO:1 was found to pair with one lambda light chain (SEQ ID NO:3) and two kappa light chains (SEQ ID NOS:4-5) exemplified by clone 1286-C5.

(SEQ ID NO: 3)
QSVLTQPPSVSGAPGQRVTISCTGSSSNIGAGYDVHWYQQLPGTAPKLLIYDNNNRPSG
VPDRFSGSKSGASASLAITGLQAEDEAHYYCQSYDNSLSGSVFGGGTQLTVLS
(SEQ ID NO: 4)
DIQLTQSPSSLSASVGDRVTLTCQASQDIRKFLNWYQQKPGKGPKLLIYDASNLQRGVP
SRFSGGGSGTDFTLIISSLQPEDVGTYYCQQYDGLPFTFGGGTKLEIK
(SEQ ID NO: 5)
DIQLTQSPSSLSASIGDRVTITCQASQDIRNSLNWYEHKPGKAPKLLIHDASNLETGVPSR
FSGGGSGTDFTLTISSLQPEDFATYYCQQANSFPLTFGGGTKVEIK

The underlined hypervariable CDR regions are shown for the light chains as follows.

(SEQ ID NO: 13)
IGAGYDVHWY
(SEQ ID NO: 14)
LLIYDNNNRP
(SEQ ID NO: 15)
QSYDNSLSGS
(SEQ ID NO: 16)
IRKFLNWY
(SEQ ID NO: 17)
LLIYDASNLQ
(SEQ ID NO: 18)
QQYDGLPF
(SEQ ID NO: 19)
IRNSLNWY
(SEQ ID NO: 20)
LLIHDASNLE
(SEQ ID NO: 21)
QQANSFPL

FIGS. 4-5 show the binding ability of the clone 1286-C5 antibody and another clone 1286-A11. To characterize the function of these antibodies, the corresponding IgGs were tested for the ability to bind on a panel of different antigens (FIGS. 4 and 5), as previously described (see also, Kashyap A K et al. 2008 supra). The binding ability of the clone 1286-A11 and 1286-C5 antibodies. Both 1286-A11 and 1286-C5 were capable of binding H1N1, H3N2 and H9N2 hemagglutinin. In addition, 1286-A11 was able to bind H5N1 hemagglutinin. Micro-neutralization of various influenza subtype viruses was also performed as previously described (see also, id.).

As shown in Table 2 below, each antibody was able to neutralize H1 virus replication in vitro in MDCK cells.

TABLE 2

| | | | | Neutralization |
|---|---|---|---|---|
| | Sub-type | Strain | Binding | MIC (ug/mL) |
| 1286-C5 | | | | |
| Avian | H5N1 | Vietnam/1203/04 | No | No activity |
| Influenza | | | | at 333 |
| | H9N2 | Hong Kong 1073/99 | Yes | 100 |
| Seasonal | H1N1 | New Caledonia/20/99 | Yes | 0.5 |
| Influenza | | Solomon Islands/3/06 | Yes | 0.65 |
| | H3N2 | Wisconsin/67/05 | Yes | 0.13 |
| | | Hong Kong/68 | Yes | 0.13 |
| “Asian” | H2N2 | Adachi/1/1957 | Yes | 21 |
| Influenza | | | | |
| 1286-A11 | | | | |
| Avian | H5N1 | Vietnam/1203/04 | Yes | partial activity |
| Influenza | | | | at 333 |
| Seasonal | H1N1 | New Caledonia/20/99 | Yes | 83 |
| Influenza | | Solomon Islands/3/06 | Yes | Not tested |
| | H3N2 | Wisconsin/67/05 | Yes | No activity |
| | | Hong Kong/68 | Yes | at 333 |

1286-C5 also showed the remarkable ability to neutralize H3 virus replication. 1286-A11 did not measurably neutralize an H3 sub-type virus, but did however display H5 sub-type neutralization. The mechanism of action of the 1286-C5 antibody is through hemagglutination inhibition, while the mechanism of action for 1286-A11 has not been determined.

Each of these antibodies would be optimized for increased potency and spectrum of activity through standard directed and randomized antibody optimization techniques, such as saturation mutagenesis and error-prone PCR, respectively. Presumably these antibodies would be useful if converted to various fragments, as well as monospecific and multispecific surrobodies.

To further assess the breadth of activity, we performed binding analysis on a broad panel of HA isolates by Biolayer Interferometry. Binding was observed to the following representatives of the H1, H2, H3, H9 and H12 subtypes (Table 3).

TABLE 3

| Subtype | Isolate |
|---|---|
| H1 | A/Singapore/6/1986 |
| H1 | A/Beijing/262/1995 |
| H1 | A/Solomon Islands/3/2006 |
| H2 | A/Japan/305/1957 |
| H2 | A/Adachi/2/1957 |
| H3 | A/Hong Kong/1/1968 |
| H3 | A/Panama/2007/1999 |
| H3 | A/Moscow/10/1999 |
| H3 | A/Brisbane/19/2007 |
| H3 | A/Perth/16/2009 |
| H9 | A/turkey/Wisconsin/1/1966 |
| H12 | A/duck/Alberta/60/1976 |

### Example 3

### Generating Universal Influenza Vaccines

The goal of vaccine design against heterogeneous pathogens is to identify and design effective and broadly protective antigens. In the case of influenza, considerable historical efforts have gone into the empirical testing of conserved linear sequences and regions with little success. A plausible reason for these failures is a lack of knowledge that focused responses against antigenic test articles are actual bona fide productive sites for neutralization of an antigen on the pathogen in the setting of an actual infection. For influenza one would be expect to find these bona fide solutions within the repertoires of survivors of an influenza infection. In our case we have demonstrated that certain antibodies amongst a large collection of antibodies are capable of neutralizing multiple subtypes of Influenza. Some of these antibodies neutralize influenza through classical inhibition of hemagglutination. Collectively, we expect that the design and assessment of vaccines utilizing such cross neutralizing antibodies derived from bona fide survivors would aid in the design and validity of cross reactive or “universal” influenza vaccines.

Specifically cross neutralizing monoclonal antibodies can be used in the design and validation of vaccine production processes that maintain or enhance the quality and antigenicity of cross neutralizing epitopes in current and future manufactured vaccines. Assuming that antibody binding to vaccine is reflective of structural integrity and antigenic potential, one would assess binding of cross neutralizing antibodies to such vaccine process derivatives to quantitatively assess their cross neutralizing potential.

To maximize the responses toward these universal epitopes one would create derivatives to increase immunogenicity towards these universal epitopes through adjuvants, like a spore coat or spore exosporium. Alternatively one could engineer and optimize these cross neutralizing epitopes to increase their immunogenicity through predictive models and supportive testing. In any case the resulting antigen would again be tested to insure that not only the efficiency of binding to target was maintained, but that a directed immunogenicity was accomplished. This would either involve determining the specific universal neutralizing titers contained in the serum from immunized individuals or test animals, likely by competitive ELISA. As an in vitro surrogate, one would combine the antigen-antibody binding data with that of an in vitro or in silico predictive model for immunogenicity. To further direct responses to the universal epitope one may deimmunize known non-neutralizing and non-crossreactive hemagglutinin epitopes

It is reasonable to extend this antibody into the design and validation of engineered recombinant hemagglutinin chimeras, fragments, and conformational mimics. For instance, it is well established that influenza contains many immunodominant epitopes that give rise to non-neutralizing responses. Utilizing the cross protective antibodies it is possible to assess whether antigen variants of vaccines that have been partially or fully deimmunized for these immunodominant non-neutralizing epitopes have maintained or created enhanced recognition of the universally protective epitopes.

Also as a result of these vaccine designs, one could minimize the antigen epitopes and even remove them from the context of hemagglutinin to create a conformational cross specific antigen.

The strategies outlined above detail methods to guide a response to a minimized neutralizing epitope or element. From the knowledge of such minimized elements, which are likely be conformationally dependent and exist within discontinuous sequence space, it would be possible to recreate the conformational neutralizing epitope in a combinatorial fashion within a smaller polypeptide, as described previously (see Horowitz et al. WO08/089,073) where the proximal placement of discontinuous epitopes alone, or in the context of designed structural support, can regenerate the essential properties of conformational epitopes.

In such a design we would take the conformation epitope and express them on hemagglutinin related and unrelated structural scaffolds, or even as a collection of conformational epitopes within a library that could be selected by conformationally dependent antibodies.

The reduction of discontinuous epitopes to a conformational epitope would result in an even smaller sized peptide immunogen than that possible with traditional protein engineering. Furthermore these structural epitopes may be further enhanced, reduced in size, or substituted through the use of nonpeptide mimetics. In any event, any of these conformational derivatives or mimics would be validated by one or more of SEQ ID NOS:1-6, related antibodies comprising at least one of SEQ ID NOS:1-6, or a corresponding antibody to the influenza virus of choice.

Methods and Materials. Crossreactive Influenza HAI Epitope Spore Vaccine Targets.
1. Mammalian expression of target as secreted protein or on mammalian cell.
   a. globular HA1 variant from a single isolate
   b. globular HA1 chimera from related isolates
   c. globular HA1 chimera from unrelated isolates
   d. globular HA1 chimera from related and unrelated isolates
2. Detect conformational epitope with SEQ ID NOS:1-6 antibodies or related antibodies of secreted protein or on mammalian cell
3. Transfer successful conformational antigen to spore expression
4. Test for spore binding with SEQ ID NOS:1-6 antibodies or -related antibodies
5. Assess crossreactive immunogenicity in vivo

### Example 4

### Increasing the Potency and Spectrum of Cross Subtype Neutralizing Antibodies

Based on the sequence information for the heavy and light chains of the antibodies described in Example 1, methods of mutagenesis are used to create improved mutants for testing either individually or amongst a collection in a library. Methods commonly used to introduce beneficial mutations could be saturation mutagenesis at sites responsible for binding or error-prone PCR mutagenesis throughout the regions known to be responsible for binding.

If crossreactivity and potency are insufficient because of inherent limitations of conventional antibody optimization strategies, one might consider destinational mutagenesis, amalgamated antibody libraries, or combinations of either or both of these methods with each other or with the previously mentioned conventional optimization strategies.

### Example 5

### Co-Administration of Vaccine and Antibody to Increase Potency and Spectrum of Protection

Complexes of antibody and antigen are known to potently induce responses against numerous microbial proteins and other proteins in animals. One possible explanation is that a forced uptake of the vaccine antibody complex occurs by Fc receptors on antigen presenting cells. Complexes of cross reactive antibodies with seasonal vaccines would allow for increases in potency from year to year and because the cross-reactive antibodies recognize numerous hemagglutinin antigens, this obviates the need to recreate new antibodies when new viral isolates are selected for each seasons Influenza vaccine. Furthermore, as these antibodies are directed at conserved neutralizing regions they may actually direct a more effective protective response towards these critically conserved susceptible regions when complexed with antigen. As described previously, the vaccine may be a traditional live or killed virus, recombinant protein or protein fragment, or even minimized peptide or non-peptidic conformationally epitope complexed with an antibody, antibody fragment or derivative, or Surrobody.

### Example 6

### Protective and Therapeutic Effect Against Influenza Challenge

Female 6-8 weeks old DBA/2 (Charles River) mice were housed 5-6 per cage in ABSL3+ containment. Food and water were provided ad libitum. Mice (5-6 per group) received 1, 2.5, 10, or 25 mg antibody C05 (C05-1286) per kg of bodyweight in approximately 200-300 μL of sterile phosphate-buffered saline (PBS) by intraperitoneal (IP) injection. The control groups were injected with 200-300 μL of either 25 mg nonimmune human IgG in PBS or PBS alone via IP injection. Antibody and controls were administered 24 hours prior to viral challenge with X-31 (H3N2) or A/Memphis/3/2008 (H1N1) virus. For a lethal virus challenge, mice were inoculated by intranasal administration with 33 MLD₅₀ (50% mouse lethal dose) influenza virus in 30-50 μL of PBS. Both the H3N2 and H1N1 viruses are highly pathogenic in DBA/2 mice. Symptoms preceding death are weight loss >30% and general inactivity. Body weight, morbidity, and mortality were monitored daily for fourteen days.

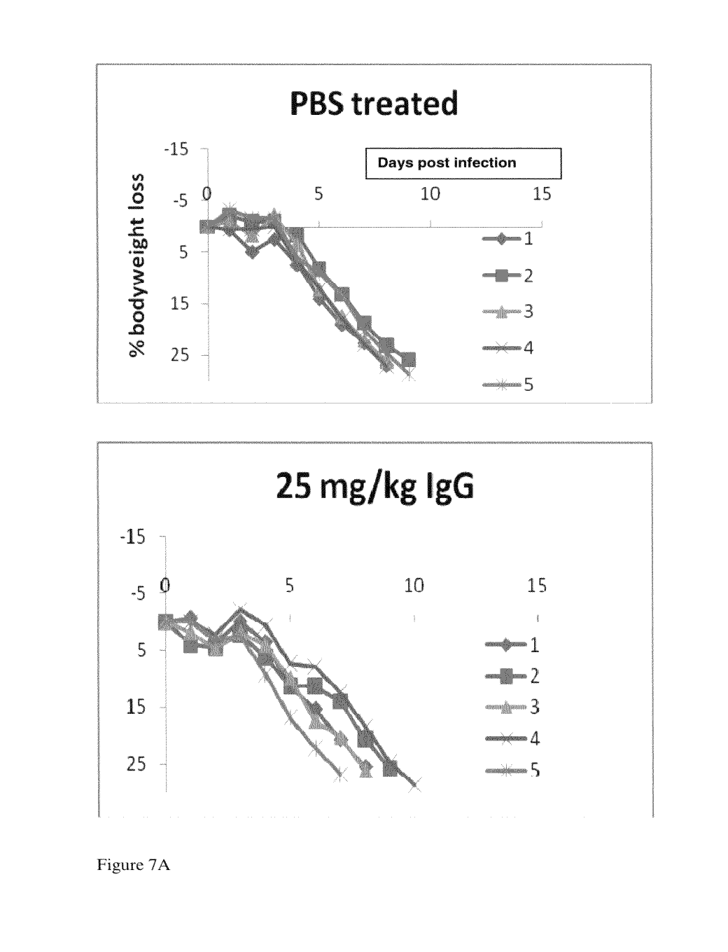

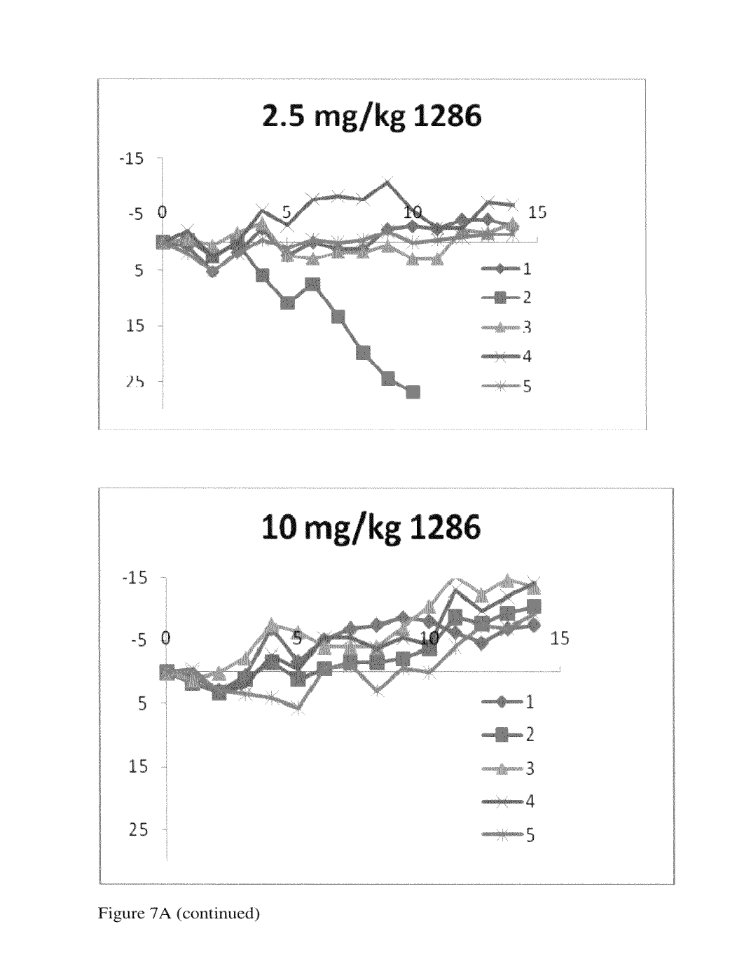

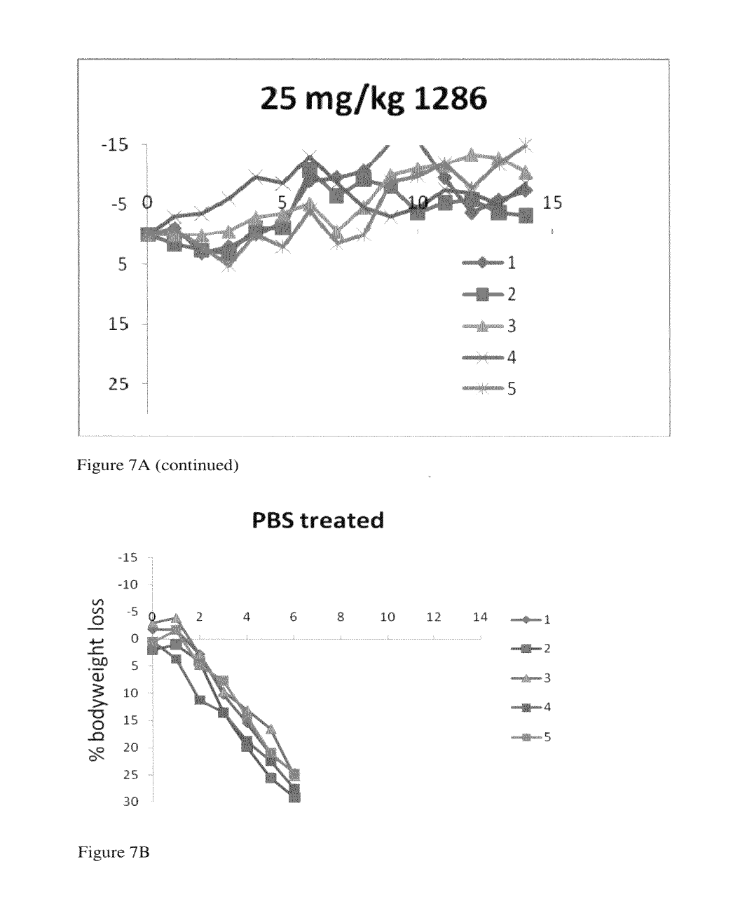

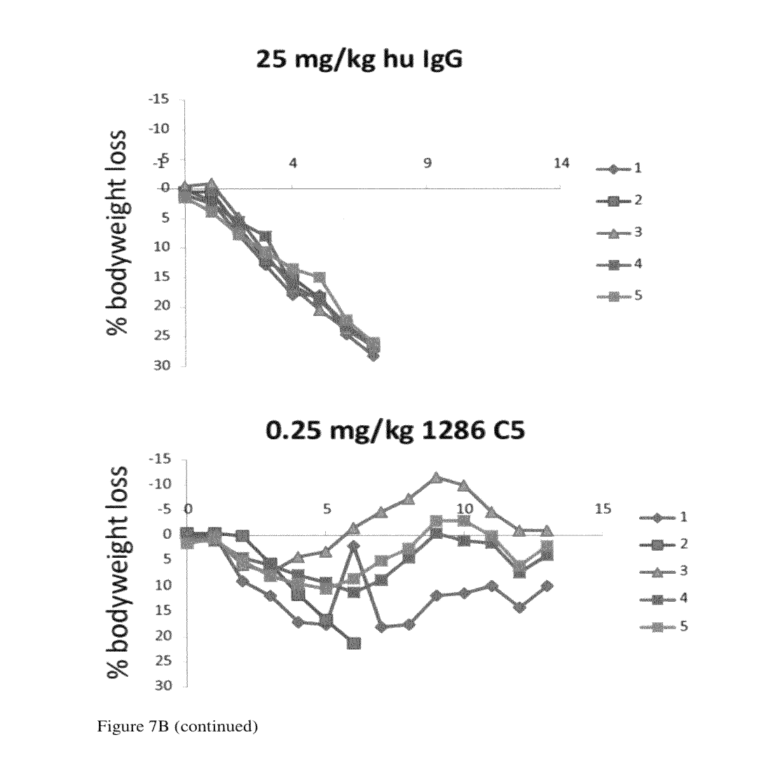

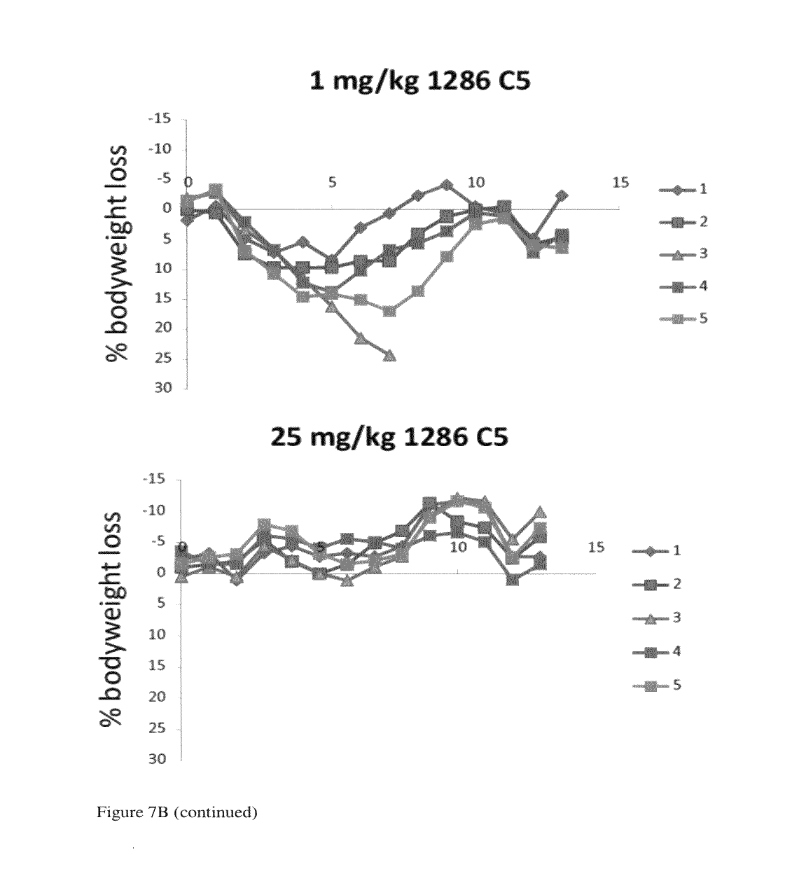

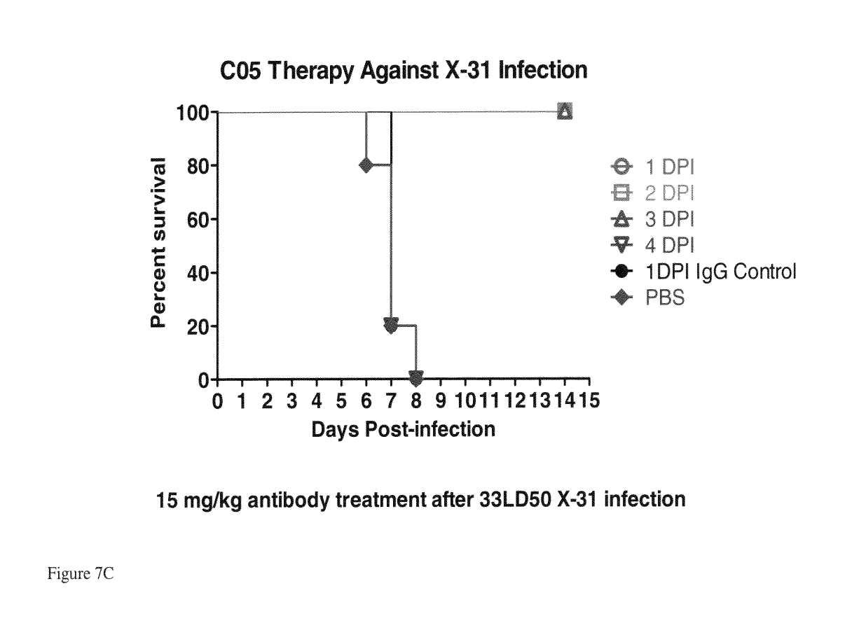

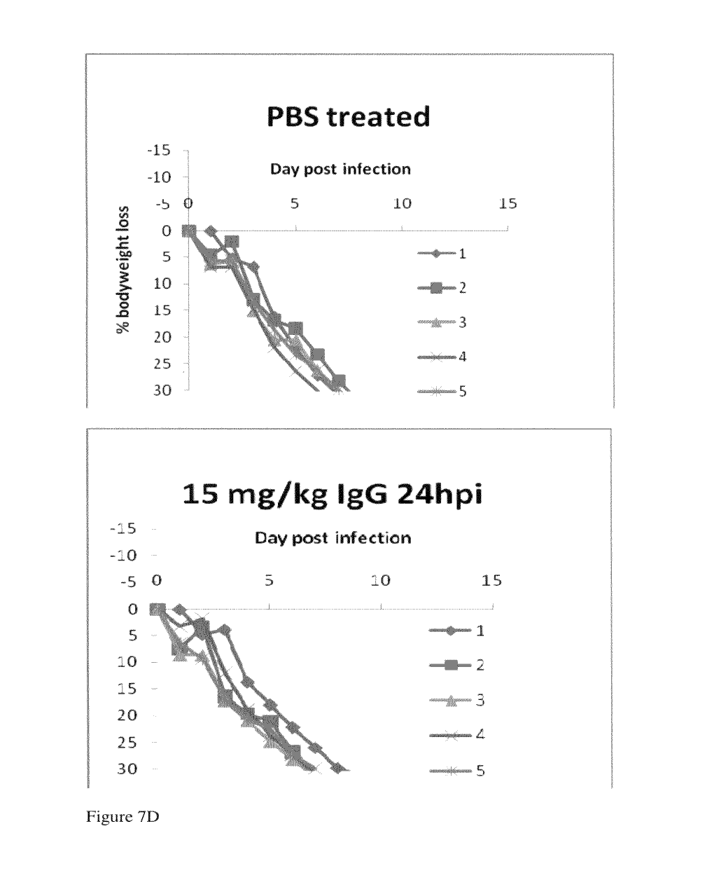

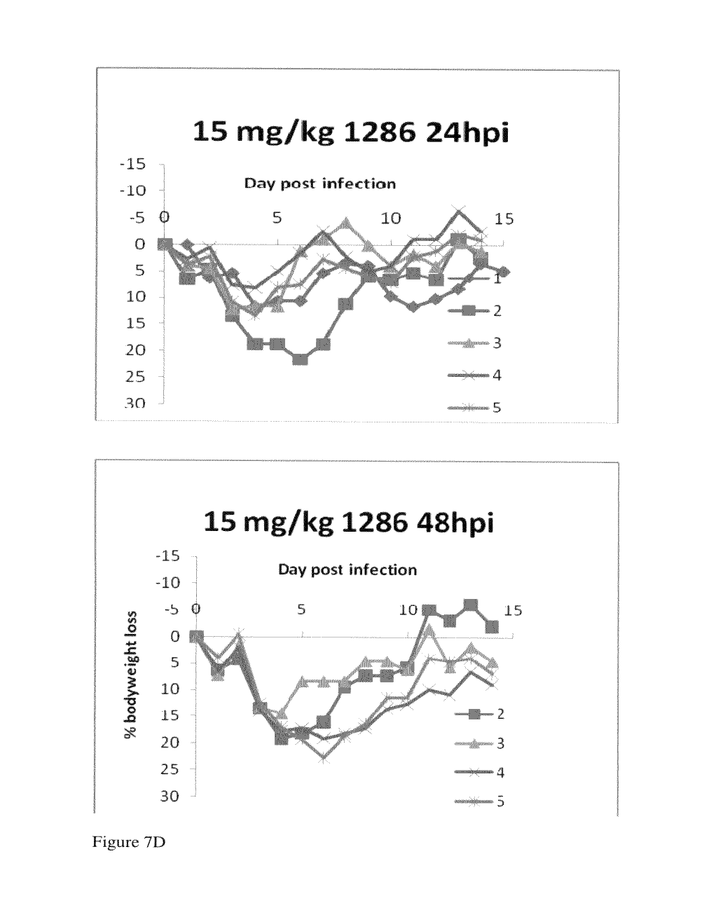

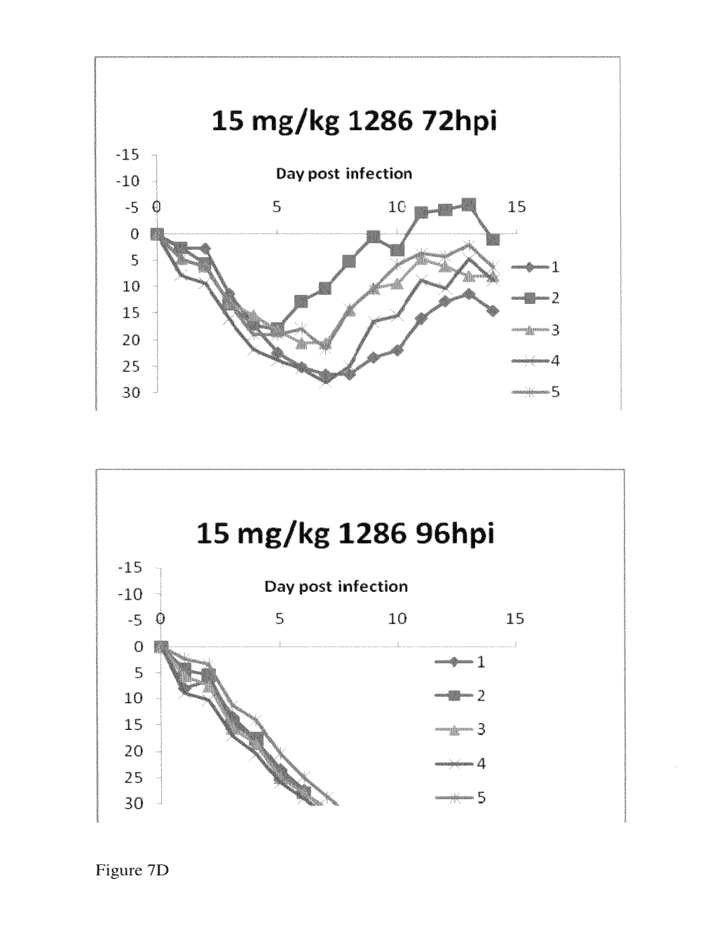

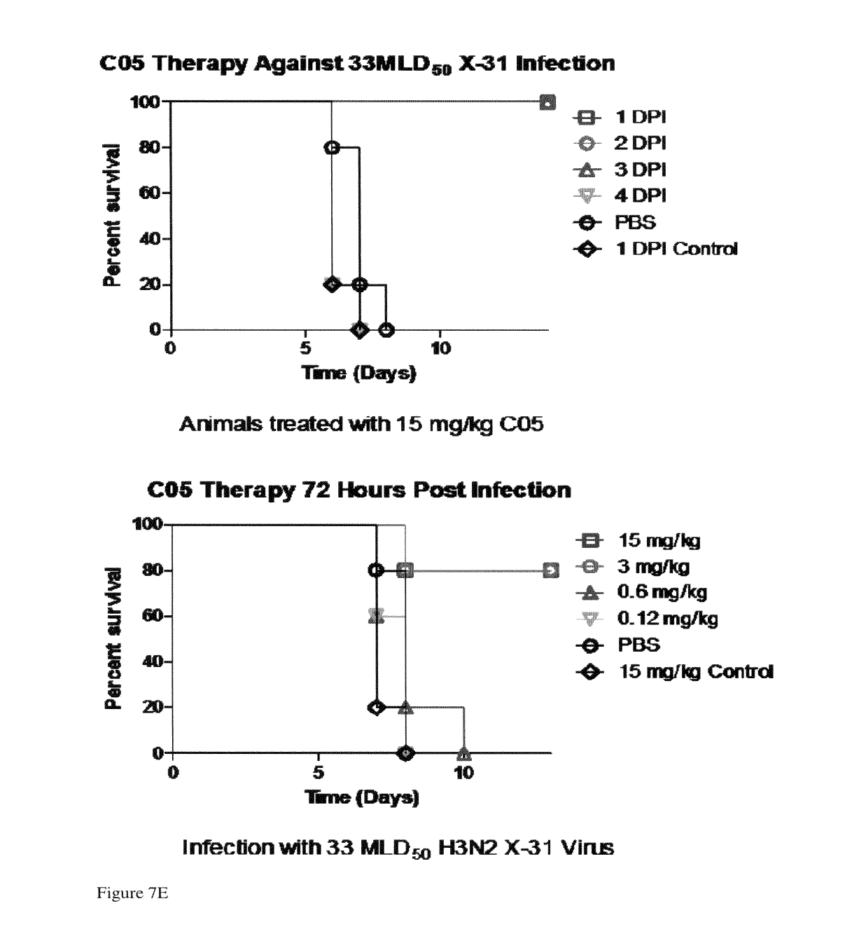

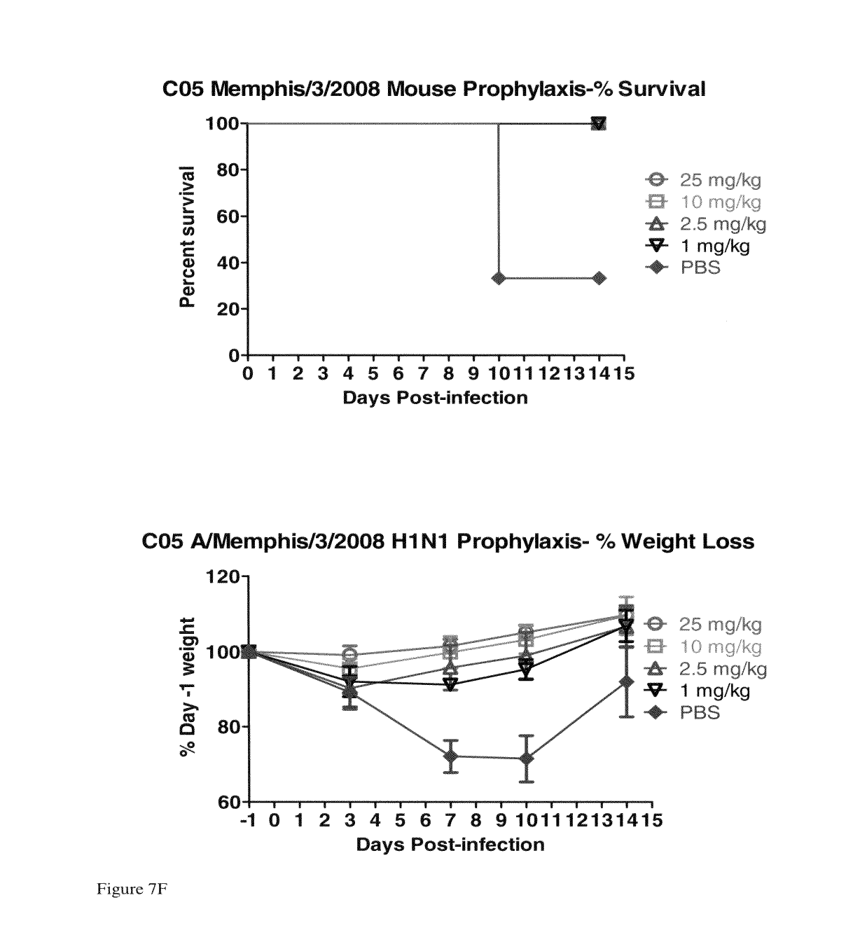

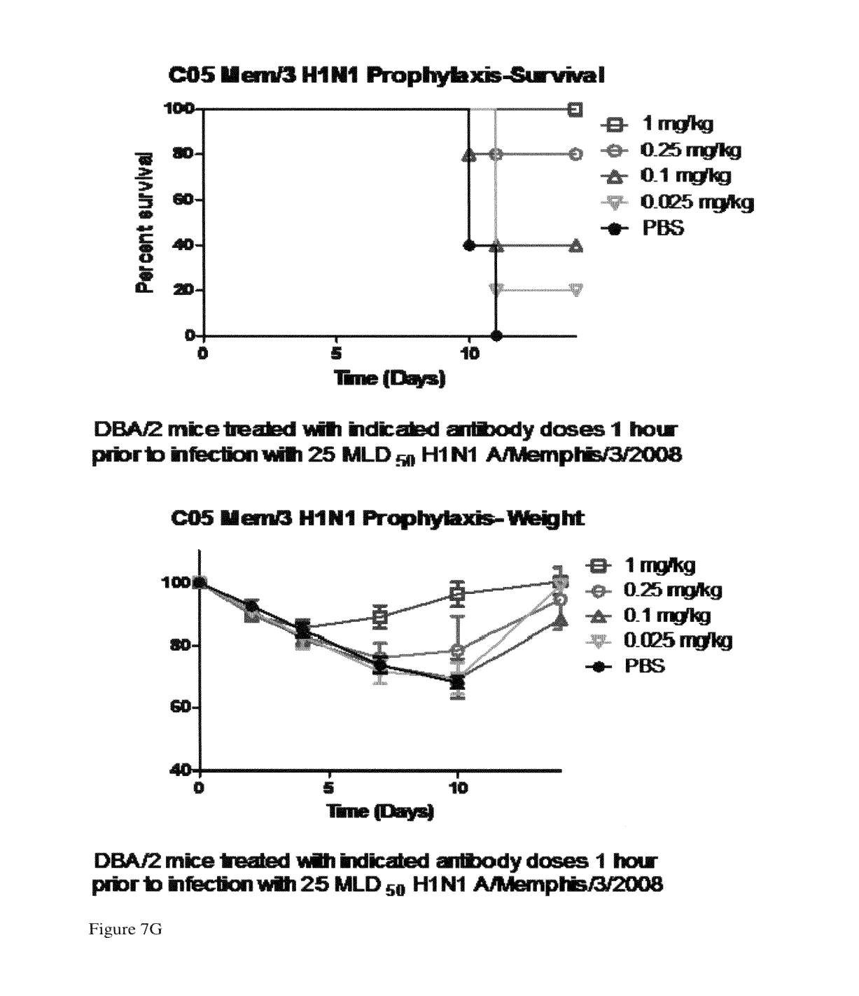

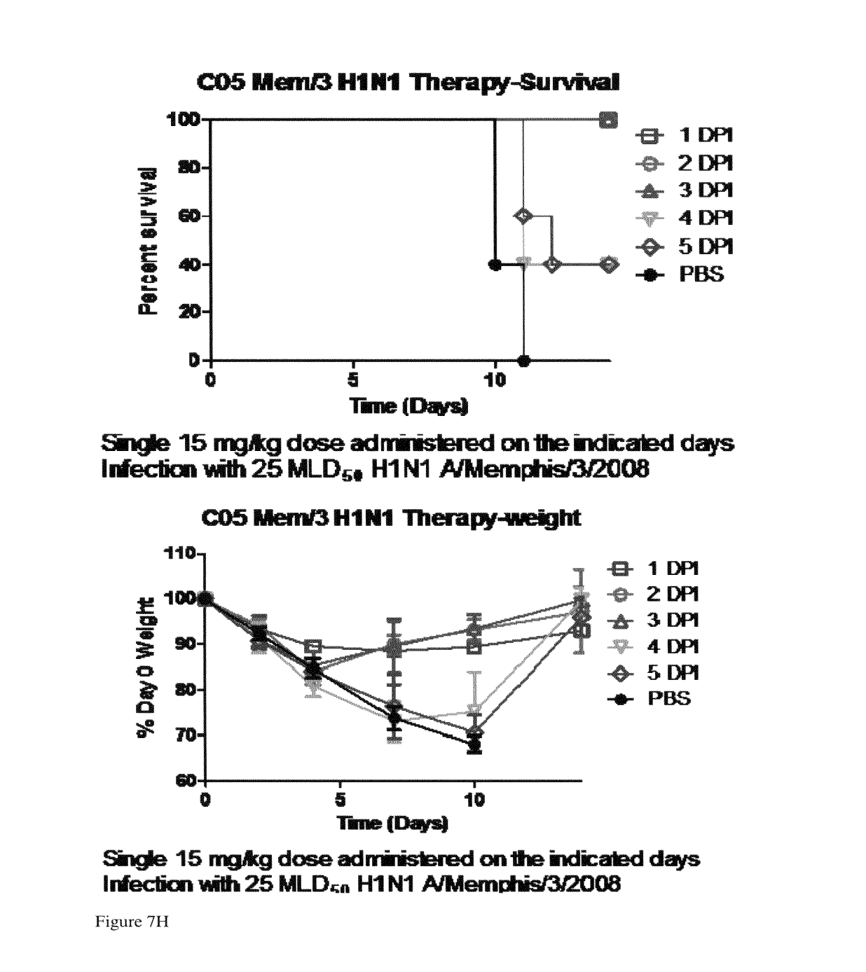

FIG. 7A illustrates the prophylactic effect of the C05 antibody against high titer lethal H3N2 viral challenge. For the high titer challenge (33 MLD₅₀), the following results were obtained. PBS treated: 0% survival. 2.5 mg/kg treated: 80% survival. 10 mg/kg treated: 100% survival. 25 mg/kg treated: 100% survival. 25 mg/kg IgG isotype: 0% survival.

The prophylactic effect of lower doses of the C05 antibody was examined in a separate study. FIG. 7B illustrates the prophylactic effect of the C05 antibody against high titer lethal H3N2 viral challenge (33 MLD₅₀) with the following results: PBS treated: 0% survival. 0.25 mg/kg treated: 80% survival; 1 mg/kg treated: 80% survival; 25 mg/kg treated: 100% survival; and 25 mg/kg IgG isotype: 0% survival. Antibodies were administered 24 hours prior to infection.

FIG. 7C-D shows a therapeutic effect by the C05 antibody against lethal H3N2 viral challenge. DBA/2 mice were first inoculated by intranasal administration with 33 MLD₅₀ (50% mouse lethal dose) X-31 (H3N2) influenza virus in 30-50 μL of PBS. Mice (5-6 per group) received 15 mg antibody C05 (C05-1286) per kg of bodyweight in approximately 200-300 μL of sterile phosphate-buffered saline (PBS) by intraperitoneal (IP) injection. The control groups were injected with 200-300 μL of either 25 mg non-immune human IgG in PBS or PBS alone via IP injection. The C05 antibody was administered at 24, 48, 72, or 96 hours after infection. Administration of the controls occurred at 24 hours after infection. Body weight, morbidity, and mortality were monitored daily for fourteen days and the results are provided in FIGS. 7C-D.

Low Dose Antibody C05 Treatment Rescues Mice from Lethal H3N2 (X-31) Infection. The therapeutic effect in vivo of the C05 antibody at lower dosages was also examined. FIG. 7E (top panel) shows the effect on survival of animals treated with a single 15 mg/kg dose after 1, 2, 3, or 4 days of lethal H3N2 influenza infection (bottom panel). Mice were successfully treated and survived up to 3 days post-infection. FIG. 7E (bottom panel) shows a dose escalation study on day 3 post-infection, where 80% of mice treated with 15 mg/kg and 3 mg/kg survived but mice treated with lower doses or with negative control agents did not.

FIG. 7F illustrates the prophylactic effect of the C05 antibody against H1N1 Memphis/3/2008 viral challenge. The % survival (top panel) and % weight loss (bottom panel) are provided.

FIG. 7G illustrates the prophylactic effect of the C05 antibody at lower doses against H1N1 Memphis/3/2008 viral challenge. DBA/2 mice were treated with 1 mg/kg, 0.25 mg/kg, 0.1 mg/kg, 0.025 mg/kg, and PBS one hour prior to infection with 25 MLD₅₀. H1N1 A/Memphis/3/2008. The % survival (top panel) and % weight loss (bottom panel) are provided. 80% protection was observed at the 0.25 mg/kg dose.

FIG. 7H illustrates the therapeutic effect of the C05 antibody at lower doses against H1N1 Memphis/3/2008 viral challenge. A single 15 mg/kg dose was administered on 1, 2, 3, 4, and 5 days after lethal infection with 25 MLD₅₀ H1N1 A/Memphis/3/2008. The % survival (top panel) and % weight loss (bottom panel) are provided. When given as a single 15 mg/kg dose, the C05 antibody provides a therapeutic effect up to three days post infection.

### Example 7

### Generating Variants with Heavy Chain Loops of Varied Lengths

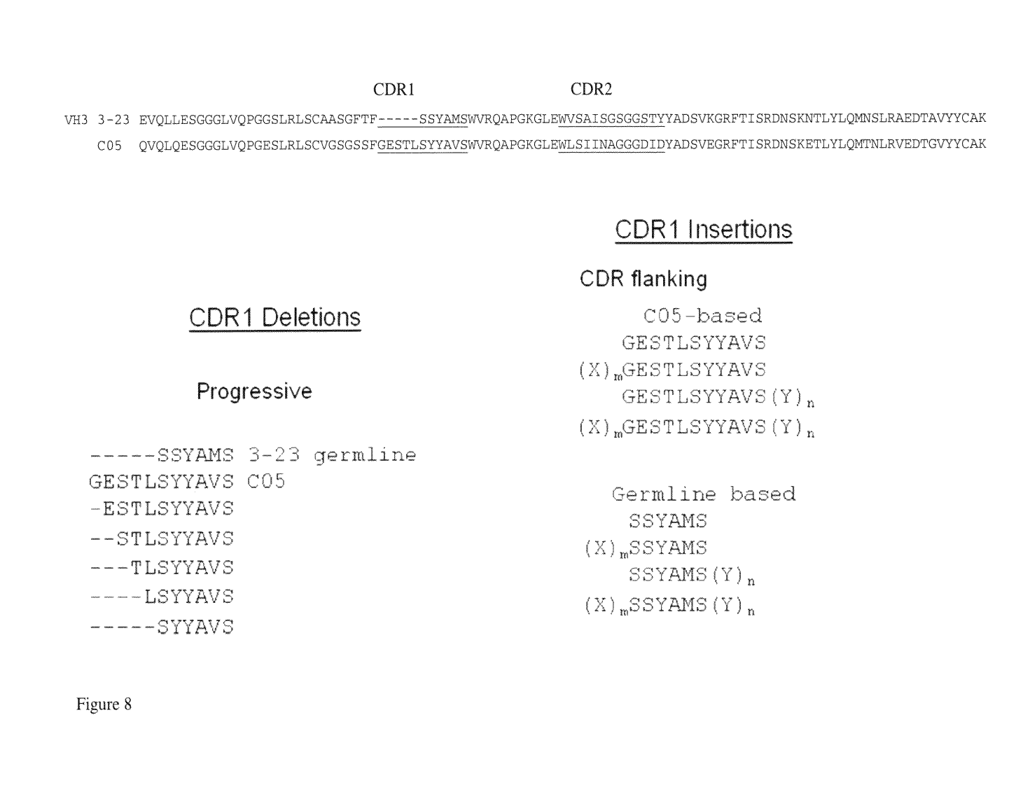

As shown in FIG. 8, the C05 antibody heavy chain sequence (SEQ ID NO:1) has a remarkably atypical length heavy chain CDR1 (SEQ ID NO:7) loop of 11 amino acids. The C05 has 5 more amino acids at CDR1, i.e., GESTL (SEQ ID NO:30), compared to VH3-23 germline.

Analysis of point mutations and truncations of the C05 heavy chain CDR 1 revealed additional requirements and tolerances in binding to HA proteins from various H3 isolates (Table 4). To determine the contribution of the elongated CDR1 in binding H3 subtype influenza HA proteins, various positions within the C05 heavy chain CDR1 were substituted with alanine or amino acids with similar hydrophobic characteristics. Additionally, targeted deletion of 5 amino acids to restore the CDR1 to the germline length was also done. Each of the resulting variants were tested by Biolayer Interferometry on a ForteBio Octet. Kinetic binding analysis was performed and the apparent affinities reported.

TABLE 4

| C05 CDR1 | | | |
|---|---|---|---|
| Variant | HK68/3 | Perth09/H3 | |
| WT | 500 nM | 18 | nM |
| del(FGEST) | 690 nM | 1300 | nM |
| F27b-A | 610 nM | 460 | nM |
| G27c-A | 450 nM | 33 | nM |
| E27d-A | 540 nM | 28 | nM |
| Y31F | 420 nM | 6.8 | nM |
| Y31L | 1400 nM | 560 | nM |
| Y31A | 640 nM | 550 | nM |

As the heavy chain CDR1 region placement is typically proximal to CDR3 and towards the edge of the binding face, it is possible that the additional length 3-5 amino acids may impart additional physical docking surface area creating greater stability for the antibody to the hemagglutinin. Analysis of the VH gene repertoire does not reveal the use of such an extended length of CDR1. Furthermore, BLAST searches do not identify any antibodies with such a CDR1 length. It therefore bears consideration that positive selection may have played a role in the existence and reinforcement of such a novel CDR1 length and its composition. Because the heavy chain is marked by such unique loop lengths in both CDR1 and CDR3 that one would consider increasing the potency and breadth of activity not only by varying the composition of these loops but by also varying the length of these loops. In terms of making improvements one could first start by deletion of the amino acid extensions in the parental CDR1 loop and even replacement with a corresponding shorter germline CDR1 peptide sequence and/or through the generation of intermediate length loops, or diversified collections thereof, as shown above (lower left panel). Still if the extended loop is beneficial, but not entirely optimal, one would consider extending the loop by insertion of (1-20) random or selected amino acids within CDR1, the FR1 junction, and/or the FR2 junctions of CDR1 in a stepwise and combinatorial fashion. By creating a combinatorial library of such antibodies with extended loop lengths one could discriminately screen for better and broader binders to both susceptible and unsusceptible influenza isolates, strains, and types.

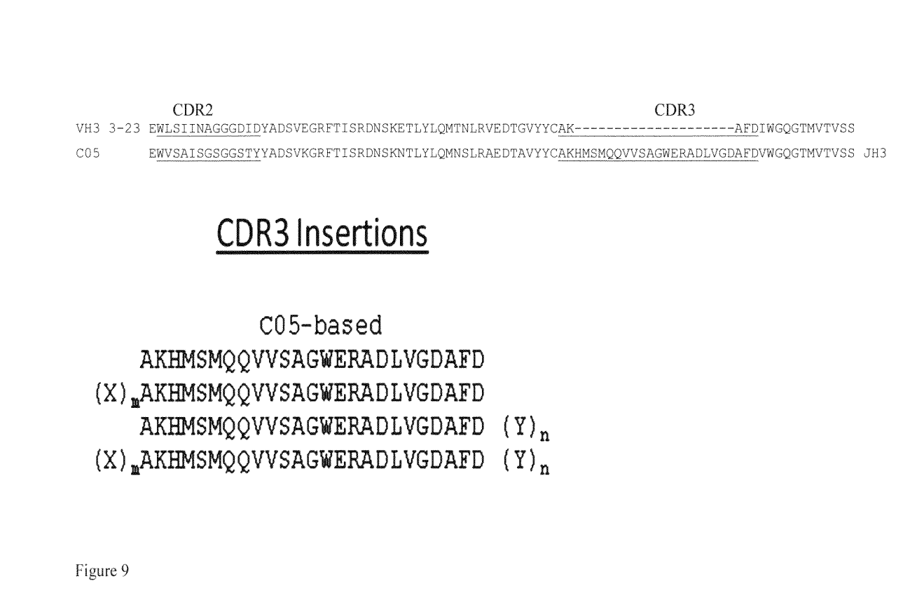

FIG. 9 shows that the C05 antibody is marked by a longer than typical heavy chain CDR3 (SEQ ID NO:9) loop of 25 amino acids. Similar to the CDR1 insertion and deletions examples described above, the CDR3 region can be similarly modified. As depicted in FIG. 9 (lower panel), the CDR3 sequences could be similarly contracted or expanded, by 1-20 amino acids, within the loops or at the FR3 and FR4 junctions of CDR3 in a step wise combinatorial fashion (genomic sequence of C05 is shown).

By analogy to other mutually beneficial mutagenesis strategies, the varied loop length CDR1 and CDR3 libraries could be combined to interrogate novel and more broadly potent anti-influenza antibodies against the currently susceptible and unsusceptible influenza isolates, strains, and types. Importantly, these strategies could also be applied not only to the parental C05 CDRs, but also to the Vh3-23 germline or other Vh germline CDR1 loops crossed into the antibody of interest.

### Example 8

### Variants with Decreased Oxidative Heterogeneity Potential

The antibody 1286-C05 contains two methionines within the heavy chain CDR3 loop at Kabat residues 96 and 98. By definition this loop is surface exposed and therefore susceptible to oxidation. We tested whether either or both methionines were essential by generating double point mutations that substituted alanine, leucine, or serine for methionine at Kabat residue 96, and a leucine for methionine at Kabat residue 98. The corresponding proteins were produced in transient mammalian systems and purified as previously described (Kashyap A K et al. supra 2008). The resulting proteins were tested for binding to the H1 (New Caledonia/20/99) hemagglutinin and found to bind within a fold of the parental protein. Next these proteins were test for their ability to bind H3 (Wisconsin/67/2005) hemagglutinin, which showed the leucine 96/leucine 98 variant bound substantially better than the alanine 96/leucine 98 and the serine 96/leucine 98 variants.

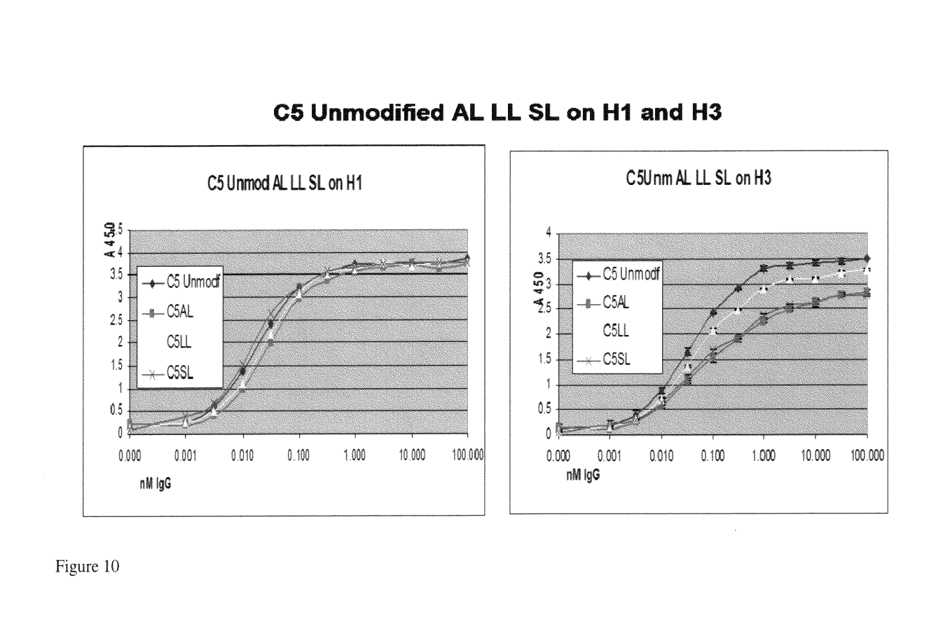

FIG. 10 illustrates that C05 nonoxidizable “XL” variants maintain recognition of H1 and H3 HA proteins.

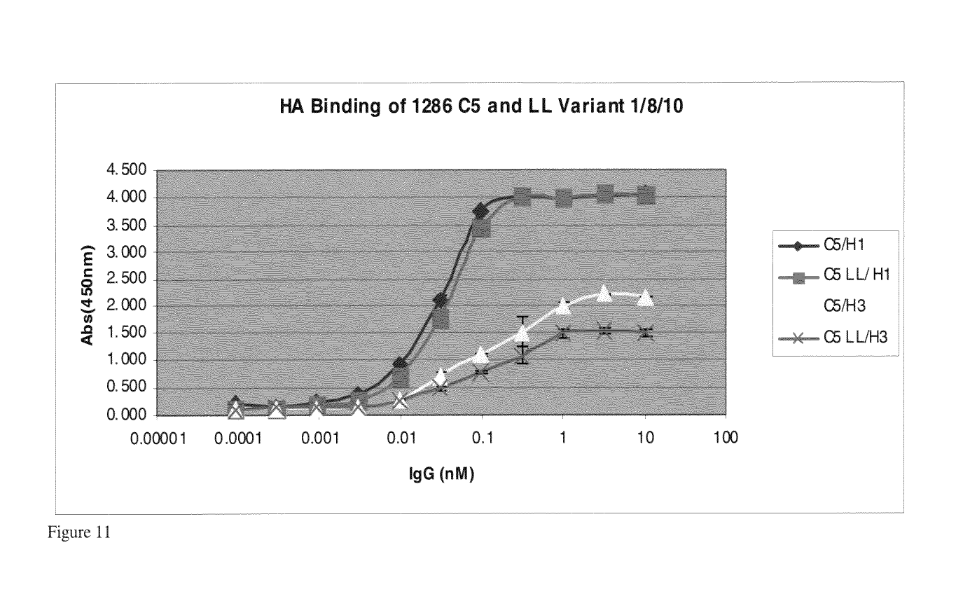

FIG. 11 shows the binding of a leucine 96/leucine 98 C05 variant to H1 (New Caledonia/20/99) and H3 (Wisconsin/67/2005) hemagglutinin, as compared to binding of non-variant C05 to H1 and H3. The C05 nonoxidizable “LL” variant maintain recognition of H1 and H3 HA proteins.

Table 3 illustrates that the C05-LL variant displayed similar potency and breadth of activity, as tested by hemagglutination inhibition (HAI) assays, where hemagglutination and the hemagglutination inhibition assays were essentially as described by Edwards and Dimmock, (Journal of Virology, v75, pp. 10208-18, 2001) and where recombinogenic virus was generated as described by Kashyap, et. al. (2008) supra. The values represent minimum concentration of antibody inhibiting hemagglutination of 0.5% cRBCs.

TABLE 3

| | | Activity with | Activity with |
|---|---|---|---|
| Subtype | Strain | 1286 C05 | 1286 C5 LL variant |
| Pandemic | (SOIV) A/Cal/04/09 | No Activity | No Activity |
| H1N1 | (6:2) | (>100 ug/ml) | (>100 ug/ml) |
| Seasonal | A/New Cal/99 | <0.1 ug/ml | 0.39 ug/ml |
| H1N1 | | | |
| | A/Texas/91 | No Activity | No Activity |
| | | (>100 ug/ml) | (>100 ug/ml) |
| | A/Bris/59/07 | 1.56 ug/ml | 3.12 ug/ml |
| | A/Sol Is/06 | 1.56 ug/ml | 3.12 ug/ml |
| | A/Virginia/87 | No Activity | No Activity |
| | | (>100 ug/ml) | (>100 ug/ml) |
| Seasonal | A/Wisc/05 | <0.1 ug/ml | <0.1 ug/ml |
| H3N2 | | | |
| | A/HK/68 | <0.1 ug/ml | 0.39 ug/ml |
| | A/Bris/10/07 | 12.5 ug/ml | 12.5 ug/ml |
| | A/Pan/99 | 0.39 ug/ml | 0.19 ug/ml |

Additional C05 CDR3 variants were generated to probe the effects on intra- and intermolecular interactions and tested for the ability to bind hemagglutinin. Intramolecular interactions are important to hold the long C05 CDR3 in the proper conformation for binding to the receptor binding pocket. Intermolecular interactions can have small or large effects on binding interactions between the antibody and hemagglutinin. Substitutions that significantly decrease binding to the receptor binding site identify critical elements required for binding and can provide a map of critical contact points in designing binding site inhibitors.

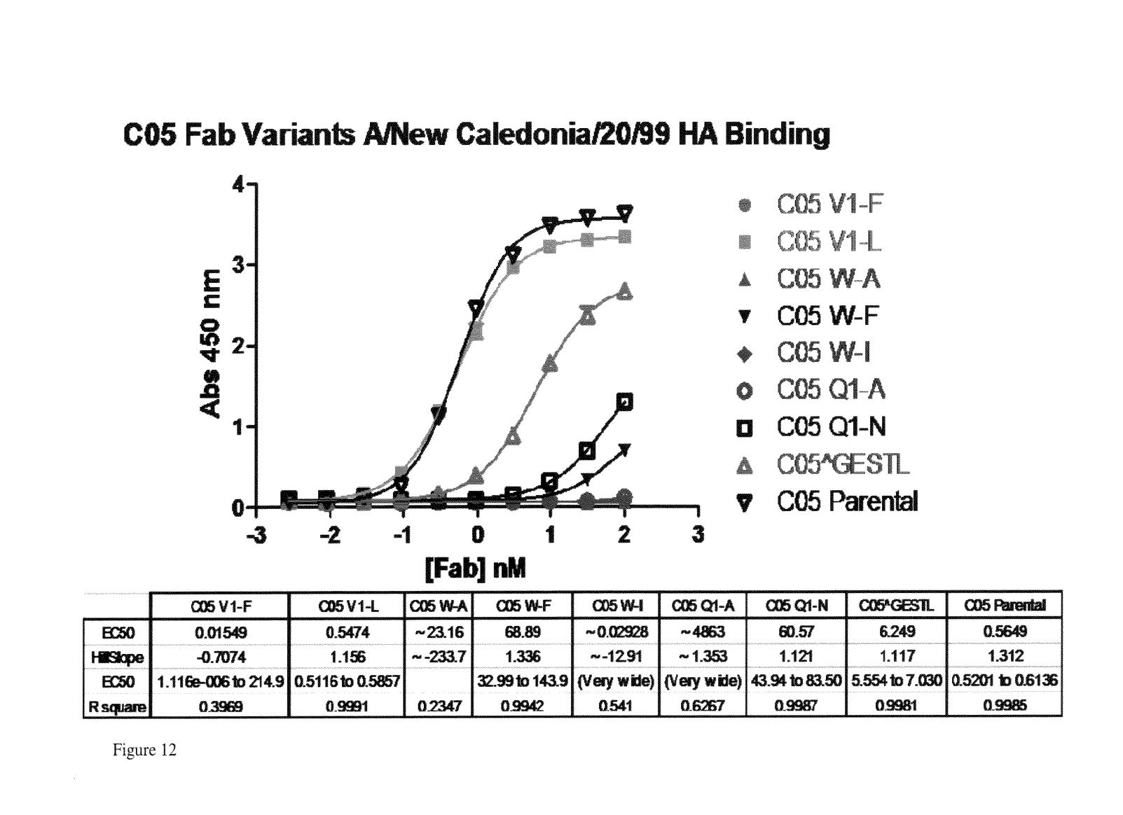

FIG. 12 shows the binding of several different C05 Fab variants of CDR3 (SEQ ID NO: 9-AKHMSMQQVVSAGWERADLVGDAFD) and CDR1 (SEQ ID NO: 7-GESTLSYYAVS) to H1 (New Caledonia/20/99) HA.

Mutations in CDR3: 1^{st} V at position 9 substituted with F; 1^{st} V at position 9 substituted with L; W at position 14 substituted with A; W at position 14 substituted with F; W at position 14 substituted with I; 1^{st} Q at position 7 substituted with A; 1^{st} Q at position 7 substituted with N.

Mutations in CDR1: Deletion of amino acids from the 1^{st} to the 5^{th} position (GESTL—SEQ ID NO:30). The C05 Parental refers to the non-mutated parent antibody.

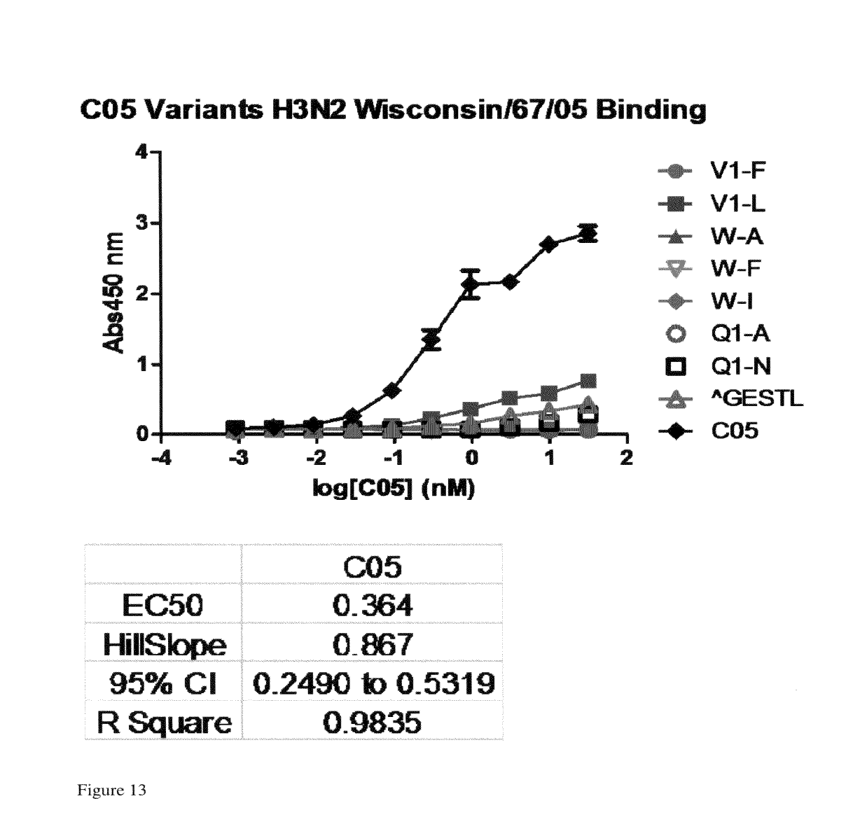

FIG. 13 shows the binding of several different C05 Fab variants of CDR3 (SEQ ID NO: 9-AKHMSMQQVVSAGWERADLVGDAFD) and CDR1 (SEQ ID NO: 7-GESTLSYYAVS) to H3 (Wisconsin/67/2005) HA.

Mutations in CDR3: 1^{st} V at position 9 substituted with F; 1^{st} V at position 9 substituted with L; W at position 14 substituted with A; W at position 14 substituted with F; W at position 14 substituted with I; 1^{st} Q at position 7 substituted with A; 1^{st} Q at position 7 substituted with N.

Although in the foregoing description the invention is illustrated with reference to certain embodiments, it is not so limited. Indeed, various modifications of the invention in addition to those shown and described herein will become apparent to those skilled in the art from the foregoing description and fall within the scope of the appended claims.

1. A method of identifying a potential influenza virus neutralizing agent, which agent mimics the binding site of an influenza virus A neutralizing molecule, wherein said molecule comprises one, two, or three hypervariable region sequences from a heavy chain selected from the group consisting of SEQ ID NO: 7, SEQ ID NO: 8, and SEQ ID NO: 9, or a functionally active fragment thereof, the method comprising
(a) employing the variant amino acid sequences of at least one heavy chain hypervariable region sequence in rational drug design to design a potential influenza virus neutralizing agent which mimics the neutralizing molecule; and
(b) contacting said potential influenza virus neutralizing agent from step (a) with an influenza virus to determine its capacity to act as a neutralizing agent.

2. The method of claim 1, wherein
(a) the agent mimics a qualitative activity of the neutralizing antibody;
(b) the hypervariable region sequence of (a) comprises a sequence selected from the group consisting of SEQ ID NO: 7; SEQ ID NO: 8, and SEQ ID NO: 9;
(c) the neutralizing agent is a Surrobody, an influenza neutralizing antibody, antibody fragment, peptide mimetic, a fusion protein, an immunoadhesin, or a small molecule; or
(d) the neutralizing molecule is a C05 antibody.

3. The method of claim 2, wherein the agent has the ability to bind influenza virus.

4-8. (canceled)

9. The method of claim 2, wherein the agent is a Surrobody, an influenza neutralizing antibody, or an antibody fragment, and comprises at least one heavy chain hypervariable region having an extended amino acid sequence as compared to its germline sequence.

10. The method of claim 9, wherein the at least one heavy chain hypervariable region is (a) HVR-H1, (b) HVR-H3, or (c) HVR-H1 and HVR-H3.

11-12. (canceled)

13. The method of claim 10, wherein
(a) the HVR-H1 extended amino acid sequence comprises about 1 to about 5 amino acids; and/or
(b) the HVR-H3 extended amino acid sequence comprises about 1 to about 20 amino acids.

14. (canceled)

15. The method of claim 13, wherein the extended amino acid sequence is GESTL (SEQ ID NO:30), or a variant thereof.

16. The method of claim 13, wherein the HVR-H3 extended amino acid sequence is
(a) HMSMQQVVSAGWERADLVGD (SEQ ID NO:31), or a variant thereof or
(b) a variant of SEQ ID NO:9.

17-22. (canceled)

23. An influenza virus neutralizing molecule comprising
a) at least one HVR sequence selected from the group consisting of:
(SEQ ID NO: 7)

| | |
|---|---|
| (i) | HVR-H1 comprising GESTLSYYAVS; |

(SEQ ID NO: 8)

| | |
|---|---|
| (ii) | HVR-H2 comprising WLSIINAGGGDID; |

(SEQ ID NO: 9)

| | |
|---|---|
| (iii) | HVR-H3 comprising AKHMSMQQVVSAGWERADLVGDAFD, |

and
b) at least one variant HVR, wherein the HVR comprises modification of at least one residue of the sequence depicted in SEQ ID NOS: 7, 8, or 9.

24. The neutralizing molecule of claim 23, wherein
(a) G in a variant HVR-H1 is A;
(b) the first Y in a variant HVR-H1 is F; or
(c) the neutralizing molecule further comprises a surrogate light chain.

25. (canceled)

26. The neutralizing molecule of claim 23, further comprising at least one HVR sequence selected from the group consisting of:

| (SEQ ID NO: 13) | |
|---|---|
| (i) | IGAGYDVHWY; |
| (SEQ ID NO: 14) | |
| (ii) | LLIYDNNNRP; |
| (SEQ ID NO: 15) | |
| (iii) | QSYDNSLSGS; |
| (SEQ ID NO: 16) | |
| (iv) | IRKFLNWY; |
| (SEQ ID NO: 17) | |
| (v) | LLIYDASNLQ; |
| (SEQ ID NO: 18) | |
| (vi) | QQYDGLPF; |
| (SEQ ID NO: 19) | |
| (vii) | IRNSLNWY; |
| (SEQ ID NO: 20) | |
| (viii) | LLIHDASNLE; |
| and | |
| (SEQ ID NO: 21) | |
| (ix) | QQANSFPL. |

27. (canceled)

28. The method of claim 1 or 2, wherein the agent identified is an antibody, and the method further comprises the step of selecting a peptide or polypeptide that functionally mimics a neutralization epitope to which said identified antibody binds.

29. The method of claim 28, wherein said identified antibody binds a hemagglutinin (HA) antigen.

30. The method of claim 28, wherein the method further comprises the developing a vaccine based upon the selected peptide or polypeptide.

31. The method of claim 30, wherein
(a) the vaccine is a synthetic vaccine;
(b) the vaccine comprises (i) an attenuated influenza A virus, or a part thereof; or (ii) a killed influenza A virus, or part thereof;
(c) the vaccine comprises a peptide or polypeptide functionally mimicking a neutralization epitope bound by an antibody that binds a hemagglutinin (HA) antigen selected from an H3 subtype and an H1 subtype; or
(d) the peptides or polypeptides of the vaccine contain antigenic determinants that raise crossreactive influenza A virus neutralizing antibodies.


SEQUENCE LISTING (68 sequences)

SEQ ID NO: 1 - protein, 138 aa, Homo sapiens
QVQLQESGGGLVQPGESLRLSCVGSGSSFGESTLSYYAVSWVRQAPGKGLEWLSIINAGGGDIDYADSVEGRFTISRDNSKETLYLQMTNLRVEDTGVYYCAKHMSMQQVVSAGWERADLVGDAFDVWGQGTMVTVSS

SEQ ID NO: 2 - protein, 127 aa, Homo sapiens
QVQLQQSGPRLVKPSQTLSLTCAISGDSVSGDSGTWNWIRQSPSRGLEWLGRTYYRSKWYNDYAESVKSRIVIKADTSKNEFSLQLNSVTPEDTAIYYCARAGVKIFGLIVGALDNWGRGTLVTVSS

SEQ ID NO: 3 - protein, 112 aa, Homo sapiens
QSVLTQPPSVSGAPGQRVTISCTGSSSNIGAGYDVHWYQQLPGTAPKLLIYDNNNRPSGVPDRFSGSKSGASASLAITGLQAEDEAHYYCQSYDNSLSGSVFGGGTQLTVLS

SEQ ID NO: 4 - protein, 107 aa, Homo sapiens
DIQLTQSPSSLSASVGDRVTLTCQASQDIRKFLNWYQQKPGKGPKLLIYDASNLQRGVPSRFSGGGSGTDFTLIISSLQPEDVGTYYCQQYDGLPFTFGGGTKLEIK

SEQ ID NO: 5 - protein, 107 aa, Homo sapiens
DIQLTQSPSSLSASIGDRVTITCQASQDIRNSLNWYEHKPGKAPKLLIHDASNLETGVPSRFSGGGSGTDFTLTISSLQPEDFATYYCQQANSFPLTFGGGTKVEIK

SEQ ID NO: 6 - protein, 109 aa, Homo sapiens
EIVMTQSPGTLSLSPGERATLSCRASQSVSSSYLAWYQQKPGQAPRLLIYGASTRATGVPDRFHGGGSGTDFTLTISRLEPEDFAVYYCQQYDTSSGLTFGGGTKVEIK

SEQ ID NO: 7 - protein, 11 aa, Homo sapiens
GESTLSYYAVS

SEQ ID NO: 8 - protein, 13 aa, Homo sapiens
WLSIINAGGGDID

SEQ ID NO: 9 - protein, 25 aa, Homo sapiens
AKHMSMQQVVSAGWERADLVGDAFD

SEQ ID NO: 10 - protein, 8 aa, Homo sapiens
SGDSGTWN

SEQ ID NO: 11 - protein, 14 aa, Homo sapiens
WLGRTYYRSKWYND

SEQ ID NO: 12 - protein, 16 aa, Homo sapiens
ARAGVKIFGLIVGALD

SEQ ID NO: 13 - protein, 10 aa, Homo sapiens
IGAGYDVHWY

SEQ ID NO: 14 - protein, 10 aa, Homo sapiens
LLIYDNNNRP

SEQ ID NO: 15 - protein, 10 aa, Homo sapiens
QSYDNSLSGS

SEQ ID NO: 16 - protein, 8 aa, Homo sapiens
IRKFLNWY

SEQ ID NO: 17 - protein, 10 aa, Homo sapiens
LLIYDASNLQ

SEQ ID NO: 18 - protein, 8 aa, Homo sapiens
QQYDGLPF

SEQ ID NO: 19 - protein, 8 aa, Homo sapiens
IRNSLNWY

SEQ ID NO: 20 - protein, 10 aa, Homo sapiens
LLIHDASNLE

SEQ ID NO: 21 - protein, 8 aa, Homo sapiens
QQANSFPL

SEQ ID NO: 22 - protein, 7 aa, Homo sapiens
SSYLAWY

SEQ ID NO: 23 - protein, 10 aa, Homo sapiens
LLIYGASTRA

SEQ ID NO: 24 - protein, 9 aa, Homo sapiens
QQYDTSSGL

SEQ ID NO: 25 - protein, 25 aa, Homo sapiens
AKHMSLQQVVSAGWERADLVGDAFD

SEQ ID NO: 26 - protein, 25 aa, Homo sapiens
AKHLSMQQVVSAGWERADLVGDAFD

SEQ ID NO: 27 - protein, 25 aa, Homo sapiens
AKHASLQQVVSAGWERADLVGDAFD

SEQ ID NO: 28 - protein, 25 aa, Homo sapiens
AKHSSLQQVVSAGWERADLVGDAFD

SEQ ID NO: 29 - protein, 25 aa, Homo sapiens
AKHLSLQQVVSAGWERADLVGDAFD

SEQ ID NO: 30 - protein, 5 aa, Homo sapiens
GESTL

SEQ ID NO: 31 - protein, 20 aa, Homo sapiens
HMSMQQVVSAGWERADLVGD

SEQ ID NO: 32 - protein, 25 aa, Homo sapiens
AKHMSMQQFVSAGWERADLVGDAFD

SEQ ID NO: 33 - protein, 25 aa, Homo sapiens
AKHMSMQQLVSAGWERADLVGDAFD

SEQ ID NO: 34 - protein, 25 aa, Homo sapiens
AKHMSMQQVVSAGAERADLVGDAFD

SEQ ID NO: 35 - protein, 25 aa, Homo sapiens
AKHMSMQQVVSAGFERADLVGDAFD

SEQ ID NO: 36 - protein, 25 aa, Homo sapiens
AKHMSMQQVVSAGIERADLVGDAFD

SEQ ID NO: 37 - protein, 25 aa, Homo sapiens
AKHMSMAQVVSAGWERADLVGDAFD

SEQ ID NO: 38 - protein, 25 aa, Homo sapiens
AKHMSMNQVVSAGWERADLVGDAFD

SEQ ID NO: 39 - protein, 6 aa, Homo sapiens
SYYAVS

SEQ ID NO: 40 - protein, 11 aa, Artificial Sequence
  Description of Artificial Sequence Synthetic peptide
GESTLSFYAVS

SEQ ID NO: 41 - DNA, 18 bp, Artificial Sequence
  Description of Artificial Sequence Synthetic primer
tttttttttttttttttt

SEQ ID NO: 42 - protein, 6 aa, Artificial Sequence
  Description of Artificial Sequence Synthetic 6xHis tag
HHHHHH

SEQ ID NO: 43 - protein, 10 aa, Artificial Sequence
  Description of Artificial Sequence Synthetic 10xHis tag
HHHHHHHHHH

SEQ ID NO: 44 - protein, 11 aa, Artificial Sequence
  Description of Artificial Sequence Synthetic peptide
AESTLSYYAVS

SEQ ID NO: 45 - DNA, 39 bp, Artificial Sequence
  Description of Artificial Sequence Synthetic oligonucleotide
acnaargcntcntayctnagyacnagyagyagyctngay

SEQ ID NO: 46 - protein, 13 aa, Artificial Sequence
  Description of Artificial Sequence Synthetic peptide
TKASYLSTSSSLD

SEQ ID NO: 47 - DNA, 39 bp, Artificial Sequence
  Description of Artificial Sequence Synthetic oligonucleotide
gcncgnggnathtayttytayggnacnacntayttygay

SEQ ID NO: 48 - protein, 13 aa, Artificial Sequence
  Description of Artificial Sequence Synthetic peptide
ARGIYFYGTTYFD

SEQ ID NO: 49 - DNA, 39 bp, Artificial Sequence
  Description of Artificial Sequence Synthetic oligonucleotide
rcnmrngsnwyntayytnwryrsnasnasnwryytngay

SEQ ID NO: 50 - protein, 13 aa, Artificial Sequence
  Description of Artificial Sequence Synthetic peptide
XXXXYXXXXXXXD

SEQ ID NO: 51 - protein, 98 aa, Homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 52 - protein, 103 aa, Homo sapiens
QVQLQESGGGLVQPGESLRLSCVGSGSSFGESTLSYYAVSWVRQAPGKGLEWLSIINAGGGDIDYADSVEGRFTISRDNSKETLYLQMTNLRVEDTGVYYCAK

SEQ ID NO: 53 - protein, 6 aa, Homo sapiens
SSYAMS

SEQ ID NO: 54 - protein, 10 aa, Homo sapiens
ESTLSYYAVS

SEQ ID NO: 55 - protein, 9 aa, Homo sapiens
STLSYYAVS

SEQ ID NO: 56 - protein, 8 aa, Homo sapiens
TLSYYAVS

SEQ ID NO: 57 - protein, 7 aa, Homo sapiens
LSYYAVS

SEQ ID NO: 58 - protein, 31 aa, Artificial Sequence
  Description of Artificial Sequence Synthetic polypeptide
XXXXXXXXXXXXXXXXXXXXGESTLSYYAVS

SEQ ID NO: 59 - protein, 31 aa, Artificial Sequence
  Description of Artificial Sequence Synthetic polypeptide
GESTLSYYAVSXXXXXXXXXXXXXXXXXXXX

SEQ ID NO: 60 - protein, 51 aa, Artificial Sequence
  Description of Artificial Sequence Synthetic polypeptide
XXXXXXXXXXXXXXXXXXXXGESTLSYYAVSXXXXXXXXXXXXXXXXXXXX

SEQ ID NO: 61 - protein, 26 aa, Artificial Sequence
  Description of Artificial Sequence Synthetic peptide
XXXXXXXXXXXXXXXXXXXXSSYAMS

SEQ ID NO: 62 - protein, 26 aa, Artificial Sequence
  Description of Artificial Sequence Synthetic peptide
SSYAMSXXXXXXXXXXXXXXXXXXXX

SEQ ID NO: 63 - protein, 46 aa, Artificial Sequence
  Description of Artificial Sequence Synthetic polypeptide
XXXXXXXXXXXXXXXXXXXXSSYAMSXXXXXXXXXXXXXXXXXXXX

SEQ ID NO: 64 - protein, 68 aa, Homo sapiens
EWLSIINAGGGDIDYADSVEGRFTISRDNSKETLYLQMTNLRVEDTGVYYCAKAFDIWGQGTMVTVSS

SEQ ID NO: 65 - protein, 88 aa, Homo sapiens
EWVSAISGSGGSTYYADSVKGRFTISRDNSKNTLYLQMNSLRAEDTAVYYCAKHMSMQQVVSAGWERADLVGDAFDVWGQGTMVTVSS

SEQ ID NO: 66 - protein, 45 aa, Artificial Sequence
  Description of Artificial Sequence Synthetic polypeptide
XXXXXXXXXXXXXXXXXXXXAKHMSMQQVVSAGWERADLVGDAFD

SEQ ID NO: 67 - protein, 45 aa, Artificial Sequence
  Description of Artificial Sequence Synthetic polypeptide
AKHMSMQQVVSAGWERADLVGDAFDXXXXXXXXXXXXXXXXXXXX

SEQ ID NO: 68 - protein, 65 aa, Artificial Sequence
  Description of Artificial Sequence Synthetic polypeptide
XXXXXXXXXXXXXXXXXXXXAKHMSMQQVVSAGWERADLVGDAFDXXXXXXXXXXXXXXXXXXXX